(19) United States
(12) Patent Application Publication
(10) Pub. No.: US 2026/0345775 A1
(43) Pub. Date: Nov. 5, 2026

(54) PEGylation of CAR T Cell Therapeutics

(71) Applicant: The Trustees of the University of Pennsylvania, Philadelphia, PA (US)

(72) Inventors: Michael J. Mitchell, Philadelphia, PA (US); Ningqiang Gong, Philadelphia, PA (US)

(21) Appl. No.: 19/106,330

(22) PCT Filed: Aug. 25, 2023
(86) PCT No.: PCT/US2023/072947

§ 371 Date: Feb. 25, 2025

Related U.S. Application Data
(60) Provisional application No. 63/373,517, filed on Aug. 25, 2022.

(51) Int. Cl. A61K 40/31; A61K 35/17; A61K 40/11; A61K 40/42; A61K 47/60; A61P 35/00; C12N 5/0783

(52) U.S. Cl. CPC A61K 40/31; A61K 35/17; A61K 40/11; A61K 40/4211; A61K 47/60; A61P 35/00; C12N 5/0636; C12N 2510/00

(57) ABSTRACT

The present disclosure provides modified immune cells or precursors thereof comprising chimeric antigen receptors and surface-bound, biologically inert molecules. Also included are methods of preparing said modified immune cells and methods of treating CAR treatment-related toxicities in subjects in need thereof comprising said modified immune cells.

### CROSS-REFERENCE TO RELATED APPLICATIONS

This application claims priority under 35 U.S.C. § 119(e) to U.S. Provisional Patent Application No. 63/373,517 filed Aug. 25, 2022, which is hereby incorporated herein by reference in its entirety.

### STATEMENT REGARDING FEDERALLY SPONSORED RESEARCH OR DEVELOPMENT

This invention was made with government support under TR002776 awarded by the National Institutes of Health. The government has certain rights in the invention.

### BACKGROUND OF THE INVENTION

Chimeric antigen receptor T (CAR T)-cell immunotherapy has revolutionized cancer treatment in the clinic with six CAR T cell therapies currently approved by the US Food and Drug Administration (FDA) and a wide variety of candidates under development. CAR T cells targeting CD19 in particular have shown remarkable anti-tumor efficacy against B cell malignancies that would otherwise have poor prognosis. However, there are several adverse side effects to CAR T-treatments, such as cytokine release syndrome (CRS) and neurotoxicity. Previous studies have shown that 20-70% of patients that receive the anti-CD19 CAR T cell therapy develop CRS. Severe CRS usually develops within 24 h after CAR T cell infusion, with symptoms such as high fever, increased levels of acute-phase proteins and respiratory and cardiovascular insufficiency. If left untreated, this can lead to multiple organ dysfunction or death. Often, CRS is also accompanied by neurotoxicity. However, neurotoxicity is usually delayed from days to weeks after the disappearance of CRS-related symptoms and may also induce considerable morbidity and mortality. Currently, the anti-IL-6 receptor (IL-6R) monoclonal antibody, tocilizumab, is the standard of care for CRS management in the clinic, but it generally poor at preventing delayed neurotoxicity. Thus, a need exists for safer CAR T cell therapies which can prevent or minimize the toxic side-effects of CRS and neurotoxicity. The current invention addresses this need.

### BRIEF SUMMARY

As described herein, the present invention relates in one aspect to a modified immune cell or precursor cell thereof comprising a chimeric antigen receptor (CAR) and one or more surface glycans. In certain embodiments, at least one of the surface glycans comprises a polysaccharide as described elsewhere herein.

As described herein, the present invention relates in one aspect to a method of preparing a modified immune cell or precursor cell thereof. In certain embodiments, the method comprises contacting an immune cell or precursor cell thereof comprising a chimeric antigen receptor and further comprising one or more surface glycans. In certain embodiments, at least one of the surface glycans comprises a polysaccharide as described elsewhere herein.

As described herein, the present invention relates in one aspect to a method for reducing the severity of a toxicity in a subject caused by administration of a chimeric antigen receptor (CAR) expressing T cell to the subject. In certain embodiments, the method comprises labeling the CAR expressing T cell with an azido glycan and/or a trans-cyclooctene group and/or a tetrazine group. In certain embodiments, the method comprises conjugating a spacer molecule to the azido glycan and/or trans-cyclooctene group and/or tetrazine group thereby producing a labeled CAR T cell. In certain embodiments, the conjugated spacer molecule reduces the interaction of the CAR T cell with endogenous immune cells, thereby reducing the severity of the toxicity.

As described herein, the present invention relates in one aspect to a method of treating, ameliorating, and/or preventing cancer in a subject in need thereof. In certain embodiments, the method comprises administering to the subject an effective amount of a modified immune cell or precursor thereof comprising a chimeric antigen receptor (CAR) and an azido glycan and/or a trans-cyclooctene group and/or a tetrazine group, wherein the CAR is specific for a cancer-related antigen.

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1 is a diagram illustrating that in situ PEGylation of CAR T cells alleviates cytokine release syndrome and neurotoxicity. Azide-modified CAR T cells were prepared and infused into mice (a). Similar to regular CAR T cells, they can recognize tumor cells, induce tumor cell lysis, and activate monocytes through direct cell-to-cell interactions. Over-activated monocytes secrete many toxic cytokines, which largely contributes to CRS (b). Dibenzocycloctyne (DBCO)-PEG600k is administered upon the first signs of CRS, which conjugates to the surface of CAR T cells through azide-alkyne click chemistry (c). DBCO-PEG600k thus forms a polymeric spacer on the surface of CAR T cells to block cell-to-cell interactions between CAR T cells, tumor cells and monocytes (d). This prevents CAR T cells and monocytes from expanding too quickly and becoming over-activated. Over time, small tumor antigens can reach CAR T cells to slowly activate and expand them (e). The PEG600k spacer on CAR T cell surface then becomes diluted and cell-to-cell interactions are gradually restored. CAR T cell-tumor cell interactions are restored earlier than CAR T cell-monocyte contacts because of the relatively smaller size of B cell lymphoma cells compared to monocytes (f). This creates a therapeutic window for tumor cell killing without inducing monocyte over-activation. In this way CAR T cells completely clear tumor cells without inducing severe CRS and neurotoxicity (g).

FIGS. 2A-2E illustrate that PEGylation of CAR T cells alters cell-to-cell interactions and cytokine release. Schematic showing how conjugation of DBCO-PEG to azido-glycan modified CAR T cells affects cell-to-cell interactions (FIG. 2A). Cell culture media containing DBCO-PEGs of three different molecular weights were mixed with 1 million azide-labeled CAR T cells for 1 h at 37 °C. Free DBCO-PEG was removed by washing cells three times with fresh medium. PEGylated CAR T cells (a) were co-cultured with both Raji cells (b) and monocytes (c) for 4 h and cells were examined under confocal microscopy (FIG. 2B). Raji target cell viability (FIG. 2C), monocyte IL-6 cytokine release (FIG. 2D), and T cell TNF-α cytokine release (FIG. 2E) in the supernatant was measured. Data in FIGS. 2C, 2D, and 2E were shown as the mean±s.d. (n=5) from independent experiments. ****P<0.0001, analyzed by two-tailed unpaired Student's t-test.

FIGS. 3A-3Q illustrate that in situ PEGylation of CAR T cells alleviates cytokine release syndrome. Experimental timeline shows construction of the CRS model and the following treatments (FIG. 3A). 1×10⁴ Raji cells and 1×10⁵ human CD34⁺ fetal liver cells were infused to NSG-SGM3 mice at day −35. 1×10⁶ CAR T cells were injected at day 0. Following the onset of high fever (ΔT>2 °C) (day 1), mice were intravenously injected with unmodified PEG600k, DBCO-PEG1k, or DBCO-PEG600k. Control mice were those that received a PBS injection. Mice temperature (FIG. 3B) and body weight (FIG. 3C) were monitored. Mouse blood was collected at days 3, 7, 10, 13, 15, 21, 28, and 35. The concentrations of cytokines such as IL-6 (FIG. 3D), IFN-γ (FIG. 3E), IL-1 (FIG. 3F), CXCL10 (FIG. 3G), CCL3 (FIG. 3H), and SAA (FIG. 3I) in mouse blood were determined using an ELISA assay. The numbers of human CD19⁺ cells (FIG. 3J), human CAR T cells (FIG. 3K), and human CD14⁺ monocytes (FIG. 3L) were also monitored. In order to investigate if decreased cytokine levels seen in mice treated with DBCO-PEG600k are a result of cell-to-cell interactions becoming blocked between CAR T cells, Raji tumor cells, and monocytes, a separate experiment was conducted where CAR T cells were isolated from mice and co-cultured with Raji tumor cells and monocytes. 1×10⁴ CAR T cells were co-cultured with 1×10⁴ Raji-Luc-GFP cells, and 5×10³ monocytes. After 24 h, Raji-Luc-GFP cell viability was determined (FIG. 3M). The levels of a monocyte cytokine IL-6 (FIG. 3N) and a T cell cytokine TNF-α (FIG. 3O) in the cell culture medium were detected. In order to investigate if the DBCO-PEG600k treatment can improve mouse survival, a separate animal experiment was conducted wherein Raji-Luc-GFP cells were used to construct the tumor model. Tumor-bearing mice were intravenously injected with CAR T cells at day 0. After the onset of high fever (day 1), unmodified PEG600k, DBCO-PEG1k, or DBCO-PEG600k were intravenously injected. The tumor cell burden level was monitored using IVIS imaging (FIG. 3P). Mouse survival data is shown in (FIG. 3Q). Mice were euthanized according to the following criteria: >20% body weight loss, ΔT >2 °C and serum IL-6 >1500 pg/mL. The data in FIGS. 3M, 3O, and 3P were shown as the mean±s.d. (n=5) from independent experiments. ****P<0.0001, NS, not significant, analyzed by two-tailed unpaired Student's t-test.

FIGS. 4A-41 illustrate that in situ PEGylation of CAR T cells abolishes neurotoxicity. Experimental timeline shows construction of the CRS model and the following treatments (FIG. 4A). NSG-SGM3 mice were intravenously co-infused with human fetal liver CD34⁺ cells and 1×10⁴ Raji cells at day −35. 2×10⁶ T cells from a humanized NSG-SGM3 mouse were transduced with a CD19.28z CAR and infused into tumor-bearing mice at day 0. After the onset of high fever (day 1), mice were infused with DBCO-PEG600k (10 mg/kg) or treated with a tocilizumab in vivo bio-similar antibody (10 mg/kg). Control mice were treated with PBS. Mice body temperature (FIG. 4B), weight (FIG. 4C), IL-6 (FIG. 4D), and human CD19⁺ cell levels (FIG. 4E) in mouse blood were monitored. CRS mortality curves (FIG. 4F, see Methods for CRS mortality definition) and lethal neurotoxicity curves (FIG. 4G, see Methods for lethal neurotoxicity definition). Brain H&E staining and human CD68 immunohistochemistry images of mice that received different treatments at day 37 (FIG. 4H, mice in the PBS and tocilizumab group that developed signs of neurotoxicity were used). Kaplan-Meyer survival plots are shown in FIG. 4I. Exact P values from a Mantel-Cox two-sided log-rank test are shown, for DBCO-PEG600k versus PBS, ****P=0.0004, for DBCO-PEG600k versus tocilizumab, *P=0.0449.

FIG. 5 is a diagram illustrating the mechanisms underlying CAR T cell-induced cytokine release syndrome and neurotoxicity. CAR T cells rapidly expand upon recognition of tumor cells. In addition to tumor cell killing, CAR T cells also play a role in the activation of monocytes/macrophages to induce cytokine release syndrome and neurotoxicity. Direct cell-to-cell contact between CAR T cells and monocytes/macrophages is considered to play an important role in the activation of monocytes/macrophages. Over-activation of monocytes/macrophages is a source of many toxic cytokines that induce CRS and neurotoxicity.

FIGS. 6A-6C illustrate the preparation and characterization of CAR T cells. Human T cells isolated from the spleen of humanized NSG-SGM3 mice were transduced with 1928z CAR. CAR expression was verified by flow cytometry (FIG. 6A). CD8/CD4 T cell ratios before and after CAR transduction were compared (FIG. 6B). A tumor cell killing assay was performed. CAR T cells were mixed with Raji-Luc-GFP cells at different effector (CAR T) to target cell (Raji-Luc-GFP) ratios for 24 h and the target cell viability was measured (FIG. 6C).

FIGS. 7A-7B illustrate azido glycan modification of the surface of CAR T cells. Metabolic labeling of azido-glycans on the surface of CAR T cells (FIG. 7A). The surface of CAR T cells was labeled with azido-glycan by culturing CAR T cells with Ac4ManNAz for 48 h. To demonstrate successful azido-glycan modification, Cy3-DBCO was added to the cell culture medium for 1 h and then washed with PBS. Cy3-DBCO conjugation to the cell surface was observed using confocal microscopy (FIG. 7B). Scale bar: 10 μm.

FIGS. 8A-8B illustrate the effect of azido-glycan modification on cell viability and anti-tumor efficacy of CAR T cells. CAR T cells were treated with different concentrations of azido-glycan for 48 h. Cell viability was then measured (FIG. 8A). Azido-glycan modified CAR T cells were co-cultured with target Raji-Luc-GFP cells at different effector to target cell ratios for 24 h. Target cell viability was determined using a luciferase reporter assay (FIG. 8B).

FIGS. 9A-9C illustrate the synthesis of dibenzocyclooctyne-modified PEG (DBCO-PEG). Synthesis route of DBCO-PEG (FIG. 9A), H¹-NMR (FIG. 9B) and Fourier transform infrared spectrum (FIG. 9C) of DBCO-PEG600k.

FIGS. 10A-10E illustrate the effect of PEGylation of CAR T cells on the steric hindrance of CAR T cell surfaces. Schematic describing the design of the experiment (FIG. 10A). Azido-glycan modified CAR T cells were cultured in medium containing 100 nM of different DBCO-PEGs (molecular weight 1 k, 5 k, 10 k, 100 k, 600 k) for 1 h. The cells were then washed with PBS three times. DBCO-PEG-modified CAR T cells (second to sixth cells from the left) were added to a 24-well plate that was pre-coated with anti-CD3 antibody. Unmodified CAR T cells (first cell from the left) were used as a control. After 1 h, cells that failed to bind to the anti-CD3 antibody-coated surface were removed by washing with PBS. Cells bound to the surface were imaged using confocal microscopy (FIG. 10B). The effect of DBCO-PEG modification on CAR T cell viability was measured using a luciferase reporter assay. Azido-glycan modified CAR T cells were cultured in medium containing DBCO-PEG for 1 h and washed with PBS three times. Cell viability was determined after 24 h (FIG. 10C). FIGS. 10D and 10E, The effect of DBCO-PEG600k treatment on CAR T cell viability was determined using a luciferase reporter assay (FIG. 10D) or a cell counting kit-8 (cck-8) assay (FIG. TOE). Data in (FIG. TOC), (FIG. 10D), and (FIG. TOE) were shown as mean±SD (n=5 biologically independent experiments). Statistical differences were calculated using Ordinary one-way ANOVA. P values are indicated.

FIGS. 11A-11I illustrate the construction of the humanized NSG-SGM3 mouse model. Experimental timeline for the construction of the humanized mouse model. NSG-SGM3 mice were treated with an intraperitoneal injection of busulfan (40 mg/kg) to remove the bone marrow. After 24 h, 1×10⁵ human fetal liver CD34⁺ cells were injected i.v. into mice (FIG. 11A). Humanized mouse model generation was confirmed by verifying populations of various human immune cells using flow cytometry. Cell numbers of human CD19⁺ B cells (FIG. 11B), human CD14′ monocytes (FIG. 11C), and human CD3⁺ T cells (FIG. 11D) were determined from the blood of humanized mice. Representative image of H&E stained mouse spleen (FIG. 11E), and immunohistochemistry image of mouse spleen stained with human anti-CD3 antibody (FIG. 11F). 5×10⁶ T cells from humanized NSG-SGM3 mice or T cells from a healthy human donor were i.v. injected into busulfan-treated NSG mice. Mouse body weight (FIG. 11G) and percentage of human CD3⁺ T cells in mouse blood±s.d. are shown (FIG. 11H). Flow cytometry gating strategy for characterizing the immune cell levels in FIGS. 11B-11D (FIG. 11I).

FIGS. 12A-12M illustrate the construction of the cytokine release syndrome model. Experimental timeline shows the construction of the CRS model. Co-infusion of 1×10⁴ Raji tumor cells and 1×10⁵ human CD34⁺ fetal liver cells into busulfan-treated mice occurred at day −35. 2×10⁶ CAR T cells were i.v. injected into tumor bearing mice at day 0 (FIG. 12A). Mouse body weight (FIG. 12B), and body temperature (FIG. 12C) were documented over the course of 35 days. CD19⁺ cell number (FIG. 12D), CAR T cell number (FIG. 12E), and human CD14⁺ monocyte number (FIG. 12F) per L of mouse blood were also measured. Cytokine concentrations of human IL-6 (FIG. 12G), human IL-1 (FIG. 12H), mouse SAA (FIG. 12I), human TNF-α (FIG. 12J), human IL-8 (FIG. 12K) in mouse blood were obtained. Mouse cytokine concentrations of IL-6 (FIG. 12L) and TNF-α (FIG. 12M) were also measured.

FIGS. 13A-13D illustrate the effect of tumor burden on the severity of CRS. 1×10⁵ CD34⁺ fetal liver cells were infused into busulfan-treated NSG-SGM3 mice at day −35. Mice were divided into three groups randomly. 1×10⁴ Raji cells were then infused at day −35, day −21, and day −7 to construct the high, medium and low burden tumor models, respectively. At day 0, 2×10⁶ CAR T cells metabolically labeled with azido-glycan were i.v. injected into mice. Tumor burden levels in different groups were observed at day 0 using IVIS (FIG. 13A). Mouse blood was collected and CAR T cell number per μL of blood was determined (FIG. 13B). Fold-change in CAR T cell numbers at day 7 for different groups (FIG. 13C). An in vitro experiment was performed to evaluate whether the amount of azide groups on CAR T cells after different rounds of expansion is sufficient at providing adequate steric hindrance and preventing CAR T cells from binding to an anti-CD3 antibody-coated plate. Azido-glycan modified CAR T cells were expanded to varying extents (0, 6, or 20 rounds of expansion) and were then cultured in medium containing 100 nM DBCO-PEG600k for 1 h. After washing with PBS, the cells (labeled in red) were mixed at a 1:1 ratio with unmodified CAR T cells (labeled in green). The ability of the cells to bind to an anti-CD3 antibody-coated plate was evaluated (FIG. 13D).

FIG. 14 illustrates the hemolytic analysis of mouse red blood cells after incubation with DBCO-PEG600k. Hemolysis percentage was determined by quantifying the release of hemoglobin into the buffer and then plotted as a function of DBCO-PEG600k concentration. Water and PBS were used as positive and negative controls, respectively.

FIGS. 15A-15J illustrate the optimization of the DBCO-PEG600k dose for CRS treatment in vivo. Experimental timeline shows the construction of the CRS model. 1×10⁴ Raji tumor cells and 1×10⁵ human CD34⁺ fetal liver cells were co-infused into busulfan-treated mice at day −35. 2×10⁶ Azide-labeled CAR T cells were infused at day 0. After high fever onset (day 1), DBCO-PEG600k at doses of 1, 5, 10, or 50 mg/kg were i.v. injected (FIG. 15A). Mouse body weight (FIG. 15B) and body temperature (FIG. 15C) were monitored. CAR T cell number per L of mouse blood (FIG. 15D) was documented. Mouse blood was collected at days 3, 7, 10, 13, 15, 21, 28, and 35 and cytokines such as human IL-6 (FIG. 15E), human IL-1 (FIG. 15F), and human TNF-α (FIG. 15G) were measured. FIGS. 15H-15J, PEGylated CAR T cells were sorted from mouse blood (collected at day 7) and co-cultured with 1×10⁵ Raji-Luc-GFP cells and 5×10⁴ monocytes for 24 h. Target cell killing was determined using a luciferase reporter assay (FIG. 15H). Human IL-6 (FIG. 15I) and human TNF-α (FIG. 15J) release into the culture medium was also determined.

FIGS. 16A-16I illustrate that PEGylated CAR T cells slowly expand and dilute PEG spacers to restore cell-to-cell interactions to enable cancer cell killing. 1×10⁴ Raji cells and 1×10⁵ CD34⁺ fetal liver cells were i.v. injected into humanized mice at day −35. 2×10⁶ CAR T cells were then i.v. injected at day 0. PEGylated CAR T cells were sorted from mouse blood at day 7 and co-cultured with both Raji-Luc-GFP cells and human monocytes for 4 h. Cells were observed under confocal microscopy (FIG. 16A). a, monocytes; b, Raji cells; c, CAR T cells. To investigate how the expansion of PEGylated CAR T cells affects cancer cell killing, PEGylated CAR T cells were isolated from DBCO-PEG600k-treated mice at days 10, 15, and 20. CAR T cells were then co-cultured with Raji-Luc-GFP cells for 24 h. Target cell viability was determined using a luciferase reporter assay (FIG. 16B). Human IL-6 (FIG. 16C), human IL-1 (FIG. 16D), and human TNF-α (FIG. 16E) concentrations in the culture medium were measured. Data are shown as mean±SD (n=3 biologically independent experiments). Statistical differences were calculated using two-way ANOVA with Tukey's post hoc test. P values are indicated. In order to further study the kinetics of CAR T cell activity after in situ PEGylation, another animal study was performed and mouse CAR T cells were collected more frequently (day 10, 13, 15, 17, and 20). The cells were then co-cultured with Raji-Luc-GFP cells for 24 h. Target cell viability was determined using a luciferase reporter assay (FIG. 16F). Human IL-6 (FIG. 16G), human IL-1 (FIG. 16H), and human TNF-α (FIG. 16I) concentrations in the culture medium were measured. Data are shown as mean±SD (n=3 biologically independent experiments). Statistical differences were calculated using one-way ANOVA with Tukey's post hoc test. P values are indicated.

FIGS. 17A-17G illustrate that PEG density on the surface of CAR T cells affects cell-to-cell interactions and the release of cytokines. CAR T cells were modified with DBCO-PEG600k at different concentrations (0.1, 1, 10, 100, and 1000 nM). After washing with PBS, 1×10⁴ PEGylated CAR T cells were co-cultured with 1×10⁴ Raji-Luc-GFP target cells and 5×10³ monocytes for 24 h. The cells were examined under confocal microscopy (FIG. 17A). Raji target cell viability (FIG. 17B), the release of CAR T cell-derived cytokine TNF-α (FIG. 17C), and monocyte-derived cytokines (FIGS. 17D-17G) in the supernatant were measured.

FIGS. 18A-18D illustrate that expansion of PEGylated CAR T cells by tumor antigens occurs more slowly than tumor cell-mediated CAR T cell expansion. 1×10⁶ Unmodified CAR T cells or DBCO-PEG600k-modified CAR T cells were co-cultured with 1×10⁶ Raji tumor cells or Raji tumor cell lysate. CAR T cell number was measured (FIG. 18A) and human TNF-α concentration in the cell culture medium was determined every three days (FIG. 18B). Schematic diagram shows that Raji tumor cell recognition induced rapid expansion of CAR T cells (FIG. 18C) and tumor cell lysate induced slower expansion of PEGylated CAR T cells (FIG. 18D).

FIGS. 19A-19E illustrate in situ PEGylation of CAR T cells alleviates neurotoxicity. NSG-SGM3 mice were co-infused intravenously with 1×10⁵ human fetal liver CD34⁺ cells and 1×10⁴ Raji tumor cells at day −35. 2×10⁶ CAR T cells were infused at day 0. After high fever onset (day 1), mice were infused with DBCO-PEG600k or treated with a tocilizumab in vivo bio-similar antibody. Mice treated with PBS were used as a control group. Human CAR T cell numbers per μL of blood were measured by flow cytometry (FIG. 19A). Human IFN-γ, and human IL-1 concentrations in mouse blood after CAR T cell infusion were determined by ELISA (FIGS. 19B and 19C). At around day 35 post-CAR T cell injection, humanized NSG-SGM3 mice that received either PBS or tocilizumab treatment developed paralysis (FIG. 19D) or experienced a seizure as indicated by movement along the red arrows (FIG. 19E), which are signs of lethal neurological syndrome. However, humanized NSG-SGM3 mice treated with DBCO-PEG600k did not develop paralysis and were not observed experiencing seizures indicative of lethal neurological syndrome.

FIGS. 20A-20D illustrate that the lethal neurological syndrome that developed in PBS- or tocilizumab-treated groups is not a result of xenogeneic-versus-host disease (X-GVHD). After mice in the PBS- or tocilizumab-treated groups developed paralysis or seizures, mouse skin and livers were collected for histological analysis to evaluate X-GVHD. H&E images of the skin and liver (FIGS. 20A and 20B). Immunohistochemistry analysis of human CD3⁺ T cells in the skin and liver (FIGS. 20C and 20D). Scale bar: 100 μm.

FIG. 21 depicts images of H&E-stained sections of major organs. H&E-stained sections of major organs from Raji tumor-bearing mice after different treatments. Scale bar: 100 μm.

FIGS. 22A-22G illustrate that PEGylated CAR T cells slowly expand to dilute PEG spacers on the CAR T cell surface. 1×10⁴ Raji cells and 1×10⁵ CD34⁺ fetal liver cells were i.v. injected into humanized mice at day −35. 2×10⁶ CAR T cells were subsequently i.v. injected at day 0. PEGylated CAR T cells were sorted from mouse blood at day 7 and co-cultured with both Raji-Luc-GFP cells and human monocytes for 4 h. Cells were observed under confocal microscopy (FIG. 22A). Blue, monocytes; green, Raji cells; red, CAR T cells. (FIG. 22B) and (FIG. 22C), CAR T-azide cells were labeled with carboxyfluorescein succinimidyl ester (CSFE) before infusing them into mice on day 0. CAR T cells were isolated from DBCO-PEG600k-treated mice at days 1, 10, 15, and 20 and CSFE fluorescence in CAR T cells was analyzed (FIGS. 22B, 22C). (FIG. 22D), The cells were stained using a fluorophore-conjugated anti-PEG antibody, and cell surface PEG levels were examined using flow cytometry. (FIG. 22E) and (FIG. 22F), quantifications of (FIG. 22C) and (FIG. 22D), respectively. (FIG. 22G), 10000 CAR T cells were isolated and lysed, and the PEG levels in the mixture were determined using an ELISA kit for PEG. Data in (FIGS. 22E-22G) are shown as mean±SD (n=3 biological independent experiments). Statistical differences were calculated using one-way ANOVA with Tukey's post hoc test. P values are indicated (dark: 10 versus 1; light: 15 versus 1; grey: 20 versus 1).

FIGS. 23A-23J illustrate that in situ PEGylation-induced CRS and neurotoxicity alleviation can also be achieved using the tetrazine (Tz)-trans-cyclooctene (TCO) reaction. (FIG. 23A), A Raji tumor mouse model was constructed, and TCO-modified CAR T (CAR T-TCO) cells were i.v. infused into the mice on day 0. After the onset of high fever (ΔT>2 °C, day 1), PEG600k, Tz-PEG1k, Tz-PEG600k. or PBS were i.v. infused to mice. Tumor burden (FIG. 23B), mouse body weight (FIG. 23C), temperature (FIG. 23D), and blood IL-6 (FIG. 23E) as well as IL-1 (FIG. 23F) were monitored. (FIG. 23G) and (FIG. 23H), Brain H&E staining and human CD68 IHC images of mice that received different treatments (tissues were collected at day 37). Images are representative of three independent experiments. (FIG. 23I) CRS mortality curves. (FIG. 23J), lethal neurotoxicity curves. Data in (FIG. 23C-23F) are shown as mean±SD (n=10 biologically independent animals, two-way ANOVA with Tukey's post hoc test). P values indicated in the figure are from the comparisons at day 7 (Blue: PEG600k versus PBS; pink: Tz-PEG1k versus PBS; red: Tz-PEG600k versus PBS). Comparison in (FIG. 231) and (FIG. 23J) was conducted using a Mantel-Cox two-sided log-rank test (n=10). P values are indicated.

FIGS. 24A-24G illustrate a comparison of CAR T cell ex vivo PEGylation strategy with in situ PEGylation strategy. (FIG. 24A), A Raji tumor-bearing mouse model was constructed, and mice were treated with either ex vivo PEGylated CAR T cells or regular CAR T-azide cells at day 0. On day 1, the mice received regular CAR T-azide cells developed high fever (ΔT>2 °C), so DBCO-PEG600k was injected (in situ PEGylation). Mouse body temperature (FIG. 24B), tumor burden (FIG. 24C), and blood IL-6 levels (FIG. 24D), and CAR T cell levels (FIG. 24E) were monitored. (FIG. 24F), quantification of tumor burden in different groups at day 35. FIG. 24G, Kaplan-Meyer survival plots. Data in (FIG. 24B), (FIG. 24D), (FIG. 24E), and (FIG. 24F) are shown as mean±SD (n=5). Statistical differences in (FIG. 24B), (FIG. 24D), and (FIG. 24E) were calculated using two-way ANOVA with Tukey's post hoc test. Statistical differences in (FIG. 24F) were calculated using one-way ANOVA with Tukey's post hoc test. P values indicated in the figure are from the comparisons at day 7. Statistical differences in (FIG. 24G) was conducted using a Mantel-Cox two-sided log-rank test (n=5). P values are indicated.

FIGS. 25A-25J illustrate that DBCO-PEG600k conjugation to CAR T cells decreases monocyte activation. Raji tumor cells and human CD34⁺ fetal liver cells were co-infused into busulfan-treated mice at day −35. 2×10⁶ azide-labeled CAR T cells were infused at day 0. After high fever onset (day 1), PBS, PEG600k, DBCO-PEG1k, or DBCO-PEG600k were i.v. injected. On day 7, mouse blood was collected and the levels of IL-6⁺, IL-1⁺, and TNF-α⁺ monocytes were determined (FIGS. 25A-G). (FIG. 25B), (FIG. 25D), and (FIG. 25F), representative flow dot plots of IL-6⁺, IL-1⁺, or TNF-α⁺ monocytes, respectively. (FIG. 25C), (FIG. 25E), and (FIG. 25G) are quantifications of (FIG. 25B), (FIG. 25D), and (FIG. 25F), respectively. Monocytes were sorted and cultured for 24 h, then the IL6 (FIG. 25H), L-1 (FIG. 25I), and TNF-α (FIG. 25J) levels in the cell culture media were determined using ELISA kits. Data in (FIG. 25B), (FIG. 25D), (FIG. 25F), (FIG. 25H), (FIG. 25I), (FIG. 25J) were shown as mean±SD (n=5 biological independent experiments). Statistical differences were calculated using one-way ANOVA with Tukey's post hoc test. P values are indicated in each panel (blue: PEG600k versus PBS; pink, DBCO-PEG1k versus PBS; red, DBCO-PEG600k versus PBS).

FIGS. 26A-26G illustrate that PEGylated CAR T cells slowly expand to dilute PEG spacers on the CAR T cell surface. 1×10⁴ Raji cells and 1×10⁵ CD34⁺ fetal liver cells were i.v. injected into humanized mice at day −35. 2×10⁶ CAR T cells were subsequently i.v. injected at day 0. PEGylated CAR T cells were sorted from mouse blood at day 7 and co-cultured with both Raji-Luc-GFP cells and human monocytes for 4 h. Cells were observed under confocal microscopy (FIG. 26A). Blue, monocytes; green, Raji cells; red, CAR T cells. (FIG. 26B) and (FIG. 26C), CAR T-azide cells were labeled with carboxyfluorescein succinimidyl ester (CSFE) before infusing them into mice on day 0. CAR T cells were isolated from DBCO-PEG600k-treated mice at days 1, 10, 15, and 20 and CSFE fluorescence in CAR T cells was analyzed (FIGS. 26B, 26C). (FIG. 26D), The cells were stained using a fluorophore-conjugated anti-PEG antibody, and cell surface PEG levels were examined using flow cytometry. (FIG. 26E) and (FIG. 26F), quantifications of (FIG. 26C) and (FIG. 26D), respectively. (FIG. 26G), 10000 CAR T cells were isolated and lysed, and the PEG levels in the mixture were determined using an ELISA kit for PEG. Data in (FIGS. 26E-26G) are shown as mean±SD (n=3 biological independent experiments). Statistical differences were calculated using one-way ANOVA with Tukey's post hoc test. P values are indicated (blue: 10 versus 1; pink: 15 versus 1; red: 20 versus 1).

FIGS. 27A-27M illustrates the conjugation of DBCO-PEG600k to CAR T cells in vivo. A Cy7-labeled DBCO-PEG600k (DBCO-PEG600k-Cy7) was i.v. injected into normal mice and 10 μL of peripheral blood was collected at various time points (0 h, 6 h, 12 h, 24 h, 32 h, 36 h, 48 h, and 72 h) and the Cy7 signal in the blood was detected (FIGS. 27A, 27B), data in (FIG. 27B) were shown as mean±SD, n=3 biologically independent animals. Whole body IVIS images of mice were taken 6 h following injection (FIG. 27C). Fluorophore (CSFE)-labeled CAR T cells (CAR T-CSFE) were used to treat tumor-bearing mice and the biodistribution of CAR T cells and DBCO-PEG600k-Cy7 in vivo were examined (FIGS. 27D, 27E). Mouse blood, lymph nodes, liver, and spleen were collected before injection, 10 min, 30 min, and 60 min after injection. The conjugation of DBCO-PEG600k-Cy7 to CAR T cells was investigated using flow cytometry (FIG. 27F-27M). (FIG. 27F), (FIG. 27H), (FIG. 27J), and (FIG. 27L), representative dot plots show the conjugation of DBCO-PEG600k-Cy7 to CAR T cells at indicated time points. (FIG. 27G), (FIG. 27I), (FIG. 27K), and (FIG. 27M) show quantification of Cy7⁺ CAR T cells in different tissues. The data in (FIG. 27G), (FIG. 27I), (FIG. 27K), and (FIG. 27M) are shown as mean±SD (n=5 biologically independent animals, one-way analysis of variance (ANOVA) with Tukey's post hoc test). P values are indicated (Blue: 10 min versus before injection; pink: 30 min versus before injection; red: 60 min versus before injection).

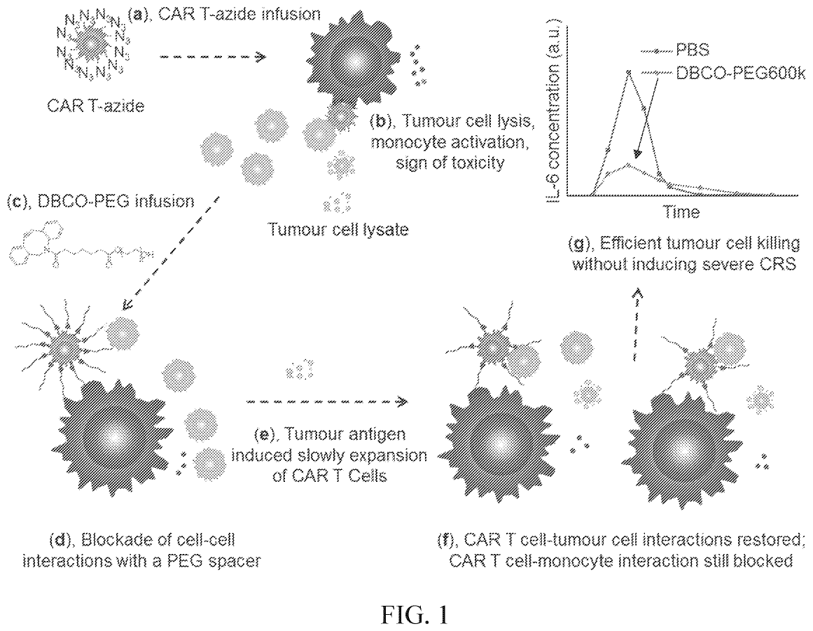

### DETAILED DESCRIPTION

The current invention is based, at least in part, on the discovery that the labeling of T cells expressing chimeric antigen receptors (CARs) with polyethylene glycol (PEG) can prevent or reduce the severity of toxicities related to CAR T cell therapy, including cytokine release syndrome and immunotherapy-related neurotoxicity. This prevention or reduction in toxicity occurs without significantly impairing the anti-tumor effects of the labeled CAR T cells.

### DEFINITIONS

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms. The term “heteroalkylene” or “heteroalkylenyl” as used herein refers to an alkylene or alkylenyl moiety, as defined herein, wherein at least one atom is a heteroatom, including but not limited to N, O, and S (e.g., —CH₂OCH₂—).

As used herein, to “alleviate” a disease means reducing the severity of one or more symptoms of the disease.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbomyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated. the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. Preferably, the epitope is about 4-18 amino acids, more preferably about 5-16 amino acids, and even more most preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present invention can be an epitope.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture.

The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

The term “glycoprotein” is herein used in its normal scientific meaning and refers to a protein comprising one or more monosaccharide or oligosaccharide chains (“glycans”) covalently bonded to the protein. A glycan may be attached to a hydroxyl group on the protein (i.e., O-linked-glycan; e.g., the hydroxyl group of serine, threonine, tyrosine, hydroxylysine) or to a nitrogen function on the protein (i.e., N-linked-glycan; e.g., asparagine or arginine) to the C1 carbon of the saccharide. A glycoprotein may comprise more than one glycan, may comprise a combination of one or more monosaccharide and one or more oligosaccharide glycans, and may comprise a combination of N-linked, O-linked and C-linked glycans

The term “glycan” is herein used in its normal scientific meaning and refers to a monosaccharide or oligosaccharide chain that is linked to a protein. The term glycan thus refers to the carbohydrate-part of a glycoprotein. The glycan is attached to a protein via the C-1 carbon of one sugar, which may be without further substitution (i.e., monosaccharide) or may be further substituted at one or more of its hydroxyl groups (i.e., polysaccharide or oligosaccharide). A naturally occurring glycan typically comprises 1 to about 10 saccharide moieties. However, when a longer saccharide chain is linked to a protein, said saccharide chain is herein also considered a glycan. A glycan of a glycoprotein may be a monosaccharide. Typically, a monosaccharide glycan of a glycoprotein consists of a single N-acetylglucosamine (GlcNAc), glucose (Glc), mannose (Man) or fucose (Fuc) covalently attached to the protein. A glycan may also be a polysaccharide or oligosaccharide. A polysaccharide or oligosaccharide chain of a glycoprotein may be linear or branched. In an oligosaccharide, the sugar that is directly attached to the protein is called the core sugar. In a polysaccharide or oligosaccharide, a sugar that is not directly attached to the protein and is attached to at least two other sugars is called an internal sugar. In a polysaccharide or oligosaccharide, a sugar that is not directly attached to the protein but to a single other sugar, i.e., carrying no further sugar substituents at one or more of its other hydroxyl groups, is called the terminal sugar or terminal polysaccharide (with reference to the terminating chain). In certain embodiments, there may exist multiple terminal sugars in an oligosaccharide of a glycoprotein, but only one core sugar.

A glycan may be an O-linked glycan, an N-linked glycan or a C-linked glycan. In an O-linked glycan a monosaccharide or oligosaccharide glycan is bonded to an O-atom in an amino acid of the protein, typically via a hydroxyl group of serine (Ser) or threonine (Thr). In an N-linked glycan a monosaccharide or oligosaccharide glycan is bonded to the protein via an N-atom in an amino acid of the protein, typically via an amide nitrogen in the side chain of asparagine (Asn) or arginine (Arg). In a C-linked glycan a monosaccharide or oligosaccharide glycan is bonded to a C-atom in an amino acid of the protein, typically to a C-atom of tryptophan (Trp). The end of an oligosaccharide that is directly attached to the protein is called the reducing end of a glycan. The other end of the oligosaccharide is called the non-reducing end of a glycan.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroalkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms, of which one or more is a heteroatom, such as but not limited to N, O, and S. Examples of straight chain heteroalkyl groups include —CH₂OCH₃, —CH₂NHCH₃, and CH₂SCH₃, inter alia.

The term “heteroalkylene” as used herein refers to a divalent radical derived from a heteroalkyl, including but not limited to, —CH₂O—, —CH₂CH₂O—, —CH₂OCH₂—, —CH₂NH—, —CH₂CH₂NHCH₂—, —CH₂NHCH₂—, and the like. For heteroalkylene groups, the one or more heteroatoms may occupy either or both terminal positions of the divalent group, or may occupy an internal position of the divalent group.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immune response” as used herein is defined as a cellular response to an antigen that occurs when lymphocytes identify antigenic molecules as foreign and induce the formation of antibodies and/or activate lymphocytes to remove the antigen.

The term “immunosuppressive” is used herein to refer to reducing overall immune response.

“Insertion/deletion”, commonly abbreviated “indel,” is a type of genetic polymorphism in which a specific nucleotide sequence is present (insertion) or absent (deletion) in a genome.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

The term “monosaccharide”, as used herein, refers to a sugar or carbohydrate of the general formula CₘH₂ₘOₘ. Examples of monosaccharides include, but not are not limited to, mannose, glucose, galactose, fructose, erythrose, threose, erythrulose, ribose, arabinose, xylose, lyxose, allose, altrose, mannose, gulose, idose, talose, ribulose, xylulose, psicose, sorbose, tagatose and Including but not limited to cedoheptulose. Monosaccharides can be naturally occurring or synthesized. Monosaccharides exist as either ring-opened monosaccharides or cyclic monosaccharides.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “oligonucleotide” typically refers to short polynucleotides. It will be understood that when a nucleotide sequence is represented by a DNA sequence (i.e., A, T, C, G), this also includes an RNA sequence (i.e., A, U, C, G) in which “U” replaces “T.”

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrastemal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “polysaccharide”, as used herein, refers to a polysaccharide comprising optionally substituted monosaccharide units linked through α-glycosidic bonds (e.g., α-1,4-glycosidic bond). The term “α-glycosidic bond” is known to those skilled in the art, and refers to a covalent bond between a hemiacetal carbon of a saccharide (e.g., monosaccharide or polysaccharide) and a hydroxyl group of a second molecule (e.g., monosaccharide or polysaccharide), preferably a saccharide. Consequently, an “α-glycosidic bond” is a bond that attaches to the atom of anomeric carbon in the alpha position, for example in the case of D-glucopyranoside in an axial orientation. One skilled in the art understands that, during the formation of a glycosidic bond, a water molecule is released, and that, therefore, for example a glucose unit involved in a glycosidic bond is preferably called anhydroglucose. Alternatively, glycosidic bonds may comprise Q-glycosidic bonds. In general, one distinguishes between α- and β-glycosidic bonds, depending on whether the substituent groups on the carbons flanking the ring oxygen are pointing in the same or opposite directions in the standard way of drawing sugars. An α-glycosidic bond for a D-sugar emanates below the plane of the sugar while the hydroxyl (or other substituent group) on the other carbon points above the plane (opposite configuration), while a β-glycosidic bond emanates above that plane (the same configuration). The alpha and beta designation is reversed for L-sugars with an opposing configuration designated beta and the same configuration designated alpha. In a 1,4-glycosidic bond a C1-O—C4 bond is made involving the C1 (hemiacetal carbon) of one sugar molecule and C4 of the other; likewise a C1-O—C6 bond is called a 1,6-glycosidic bond.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as upregulation of interferon-gamma, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a 5 peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “target site” or “target sequence” refers to a nucleic acid sequence that defines a portion of a nucleic acid to which a binding molecule may specifically bind under conditions sufficient for binding to occur. In some embodiments, a target sequence refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

“Transplant” refers to a biocompatible lattice or a donor tissue, organ or cell, to be transplanted. An example of a transplant may include but is not limited to skin cells or tissue, bone marrow, and solid organs such as heart, pancreas, kidney, lung and liver. A transplant can also refer to any material that is to be administered to a host. For example, a transplant can refer to a nucleic acid or a protein.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention provides modified immune cells or precursor cells which express chimeric antigen receptors (CARs) and have been labeled with biologically inert spacer molecules, such as polyethylene glycol, polymers, particles, or bulky biological materials, conjugated to the cell surface. These spacer molecules act to block cellular interactions with endogenous immune cells through steric hindrance. Reducing CAR T cell interaction with endogenous immune cells such as monocytes and the like reduces the release of toxic cytokines associated with CAR treatment-related toxicities, thereby preventing or reducing the severity of said CAR treatment-related toxicities. Also provided are methods of conjugating PEG molecules to the surface of CAR-expressing immune cells and methods of treatment comprising CAR expressing and spacer molecule-conjugated (e.g. PEG-conjugated) modified immune cells or precursor cells.

### Chimeric Antigen Receptors (CARs)

In certain embodiments, the current invention includes a modified immune cell or precursor thereof comprising a chimeric antigen receptor (CAR). A CAR, is a recombinant fusion protein typically comprising: an extracellular antigen-binding domain, a transmembrane domain, and an intracellular domain comprising a co-stimulatory signaling domain and/or an intracellular signaling domain.

The antigen binding domain of a CAR is an extracellular region of the CAR for binding to a specific target antigen including proteins, carbohydrates, and glycolipids. In some embodiments, the CAR comprises affinity to a target antigen (e.g. a tumor associated antigen) on a target cell (e.g. a cancer cell). The target antigen may include any type of protein, or epitope thereof, associated with the target cell. For example, the CAR may comprise affinity to a target antigen on a target cell that indicates a particular status of the target cell. CAR antigen binding domains can include any domain that binds to an antigen or epitope and may include, but is not limited to, a monoclonal antibody, a polyclonal antibody, a synthetic antibody, a human antibody, a humanized antibody, a non-human antibody, and any fragment thereof including but not limited to a Fab, a F(ab′)2, a single-domain antibody, a single-chain variable fragment (scFv), and the like.

In certain embodiments, the CAR expressed by the modified immune cells or precursor cells thereof of the invention are capable of binding specifically to antigens associated with a disease or malignancy. In certain embodiments, the disease or malignancy is cancer. Examples of cancer-related antigens that can be targeted by CARs expressed by the modified cells of the invention include, but are not limited to CD19, CD20, CD22, k light chain, CD30, CD33, CD123, CD38, ROR1, ErbB2, ErbB3/4, EGFr viii, carcinoembryonic antigen, EGP2, EGP40, mesothelin, TAG72, PSMA, NKG2D ligands, B7-H6, IL-13 receptor α2, MUC1, IL13R-alpha2, VEGF-A, Tem8, FAP, EphA2, Her2, MUC16, CA9, GD2, GD3, HMW-MAA, CD171, Lewis Y, G250/CALX, HLA-AI MAGE A1, HLA-A2 NY-ESO-1, PSC1, folate receptor-α, CD44v7/8, 8H9, NCAM, VEGF receptors, 5T4, Fetal AchR, NKG2D ligands, CD44v6, TEM1, and/or TEM8, among others. It is contemplated that the CAR expressed by the modified immune cells of the invention could be specific for any antigen associated with a disease or malignancy, in which treatment with the CAR is associated with the risk of developing a toxicity (e.g. CRS or neurotoxicity).

The transmembrane domain of a CAR can be designed to comprise a transmembrane domain that connects the antigen binding domain of the CAR to the intracellular domain. The transmembrane domain of a subject CAR is a region that is capable of spanning the plasma membrane of a cell (e.g., an immune cell or precursor thereof). The transmembrane domain is for insertion into a cell membrane, e.g., a eukaryotic cell membrane. In some embodiments, the transmembrane domain is interposed between the antigen binding domain and the intracellular domain of a CAR. The transmembrane domain may be derived either from a natural or from a synthetic source. Where the source is natural, the domain may be derived from any membrane-bound or transmembrane protein, e.g., a Type I transmembrane protein.

Where the source is synthetic, the transmembrane domain may be any artificial sequence that facilitates insertion of the CAR into a cell membrane, e.g., an artificial hydrophobic sequence. Examples of the transmembrane regions of particular use in this invention include, 5 without limitation, transmembrane domains derived from (i.e., comprise at least the transmembrane region(s) of) the alpha, beta or zeta chain of the T-cell receptor, CD28, CD2, CD3 epsilon, CD45, CD4, CD5, CD7, CD8. CD9, CD16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9. In some embodiments, the transmembrane domain may be synthetic, in which case it will comprise predominantly hydrophobic residues such as leucine and valine. In certain exemplary embodiments, a triplet of phenylalanine, tryptophan and valine will be found at each end of a synthetic transmembrane domain.

The intracellular domain of the CAR is responsible for activation of at least one of the effector functions of the cell in which the CAR is expressed (e.g., immune cell). The intracellular domain transduces the effector function signal and directs the cell (e.g., immune cell) to perform its specialized function, e.g., harming and/or destroying a target cell.

The intracellular domain or otherwise the cytoplasmic domain of the CAR is responsible for activation of the cell in which the CAR is expressed. Examples of an intracellular domain for use in the invention include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

In certain embodiments, the intracellular domain comprises a costimulatory signaling domain. In certain embodiments, the intracellular domain comprises an intracellular signaling domain. In certain embodiments, the intracellular domain comprises a costimulatory signaling domain and an intracellular signaling domain. In certain embodiments, the intracellular domain comprises CD2 and CD3 zeta. In certain embodiments, the costimulatory signaling domain comprises CD2. In certain embodiments, the intracellular signaling domain comprises CD3 zeta.

In one embodiment, the intracellular domain of the CAR comprises a costimulatory signaling domain which includes any portion of one or more co-stimulatory molecules, such as at least one signaling domain from CD2, CD3, CD8, CD27, CD28, OX40, ICOS, 4-1BB, PD-1, any derivative or variant thereof, any synthetic sequence thereof that has the same functional capability, and any combination thereof.

Examples of the intracellular signaling domain include, without limitation, the ζ chain of the T cell receptor complex or any of its homologs, e.g., η chain, FcsRIγ and B chains, MB 1 (Iga) chain, B29 (Ig) chain, etc., human CD3 zeta chain, CD3 polypeptides (Δ, δ and ε), syk 5 family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may be human CD3 zeta chain, FcγRIII, FcsRI, cytoplasmic tails of Fc receptors, an immunoreceptor tyrosine-based activation motif (ITAM) bearing cytoplasmic receptors, and combinations thereof.

Other examples of the intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fc gamma R11a, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD1Id, ITGAE, CD103, ITGAL, CD11a, LFA-1, ITGAM, CD11b, ITGAX, CD11c, ITGBl, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in a subject CAR of the present invention include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In some embodiments, the intracellular signaling domain includes at least one (e.g., one, two, three, four, five, six, etc.) ITAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in a subject CAR of the present invention include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino acids. In one embodiment, the intracellular signaling domain of a subject CAR comprises 3 ITAM motifs. In some embodiments, intracellular signaling domains includes the signaling domains of human immunoglobulin receptors that contain immunoreceptor tyrosine based activation motifs (ITAMs) such as, but not limited to, Fc gamma RI, Fc gamma RIIA, Fc gamma RIIC, Fc gamma RIIIA, FcRL5 (see, e.g., Gillis et al., Front. (2014) Immunol. 5:254).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

Modified Immune Cells In one aspect, the present disclosure provides a modified immune cell or precursor cell thereof comprising a chimeric antigen receptor (CAR) and also comprising one or more surface glycans, wherein at least one of the surface glycans comprises a polysaccharide of formula (I):

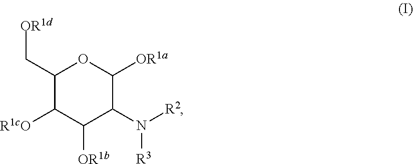

wherein:
R^{1a}, R^{1b}, R^{1c}, and R^{1d} are each independently selected from the group consisting of H, optionally substituted C₁-C₆ alkyl, optionally substituted C₂-C₆ heteroalkyl, optionally substituted C₃-C₈ cycloalkyl, optionally substituted phenyl, optionally substituted benzyl, optionally substituted C₂-C₉ heterocyclyl, C(═O)OR^{a}, C(═O)R^{a}, C(═O)N(R^{a})(R^{b}), S(═O)₂R^{a}, S(═O)₂N(R^{a})(R^{b}), a monosaccharide, a terminal polysaccharide, and a polysaccharide covalently conjugated to a CAR-T cell surface protein,
   wherein one selected from R^{1a}, R^{1b}, R^{1c}, and R^{1d} is the polysaccharide covalently conjugated to the surface protein;
R² is -L¹—Y—L²—Z;
R³ is selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
R⁴ is selected from the group consisting of optionally substituted C₁-C₆ alkyl, C₂-C₆ heteroalkyl, optionally substituted C₃-C₈ cycloalkyl, optionally substituted phenyl, optionally substituted benzyl, optionally substituted C₂-C₉ heterocyclyl, halogen, OR^{a}, N(R^{a})(R^{b}), SR^{a}, CN, and NO₂,
   wherein two adjacent R⁴ substituents may combine with the atoms to which they are bound to form an optionally substituted phenyl, optionally substituted C₃-C₈ cycloalkyl, or optionally substituted C₂-C₉ heterocyclyl;
R⁵ is selected from the group consisting of H and optionally substituted C₁-C₆ alkyl;
R⁶ is selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
L¹ is selected from the group consisting of —C(═O)(optionally substituted C₁-C₆ alkylene)-*, —C(═O)(optionally substituted C₁-C₆ heteroalkylene)-*, -(optionally substituted C₁-C₆ alkylene)-*, and -(optionally substituted C₁-C₆ heteroalkylene)-*;
L² is selected from the group consisting of **—C(═O)(optionally substituted C₁-C₁₂ alkylene)C(═O)O—, **—C(═O)(optionally substituted C₂-C₁₂ heteroalkylene)C(═O)O—, **—C(═O)(optionally substituted C₁-C₁₂ alkylene)O—, **—C(═O)(optionally substituted C₂-C₁₂ heteroalkylene)O—, **-(optionally substituted C₁-C₁₂ alkylene)C(═O)O—, **-(optionally substituted C₂-C₁₂ heteroalkylene)C(═O)O—, **-(optionally substituted C₁-C₁₂ alkylene)O—, and **-(optionally substituted C₂-C₁₂ heteroalkylene)C(═O)O—;
Y is selected from the group consisting of

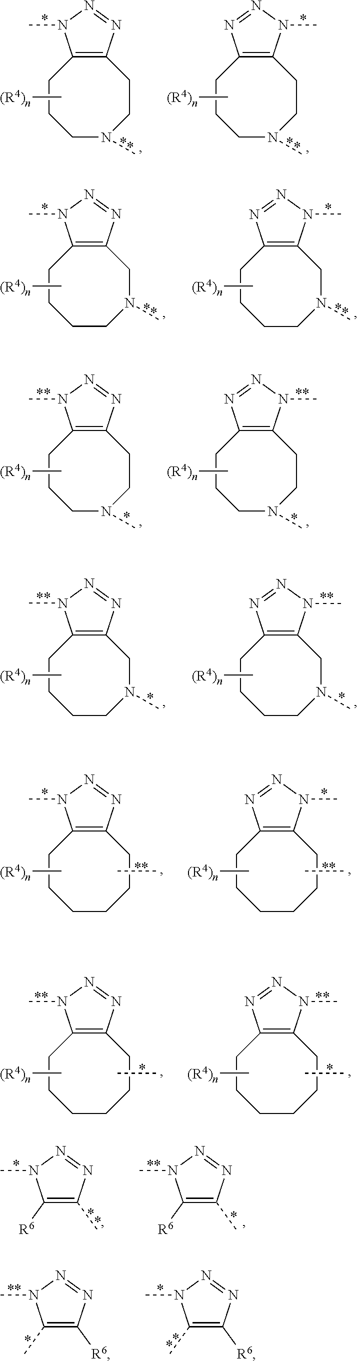

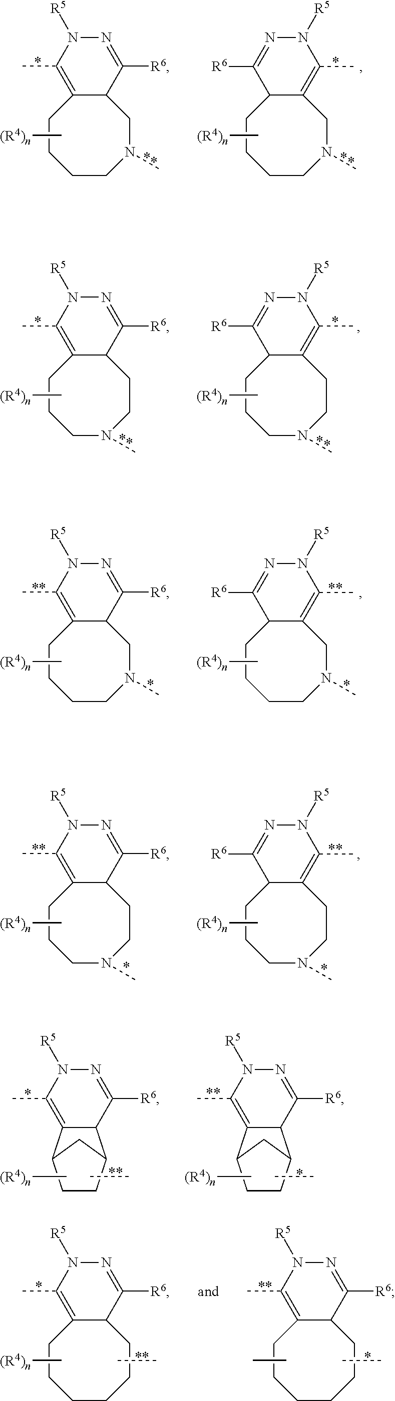

Z is selected from the group consisting of an optionally substituted heteroalkyl group, a dendrimer, a microparticle, a nanoparticle, an alignate, a biomolecule, and a modified red blood cell,
   wherein the dendrimer, microparticle, nanoparticle, alignate, biomolecule, or modified red blood cell can be covalently linked to L² by an optionally substituted heteroalkylene, and
   wherein each heteroalkyl or heteroalkylene has a molecular weight ranging from about 1 kDa to about 600 kDa;
each occurrence of R^{a} and R^{b} is independently selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
n is an integer ranging from 0 to 11;
* indicates a bond between L¹ and Y; and
** indicates a bond between Y and L².

In certain embodiments, each occurrence of optionally substituted alkyl, optionally substituted heteroalkyl, optionally substituted cycloalkyl, optionally substituted alkoxy, optionally substituted phenyl, optionally substituted benzyl, optionally substituted heterocyclyl, optionally substituted alkylene, and optionally substituted heteroalkylene, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₉ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

In certain embodiments, the compound of formula (I) is a compound of formula (Ia):

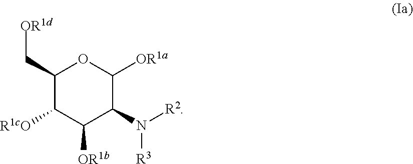

In certain embodiments, R^{1a} is the polysaccharide covalently conjugated to the surface protein, and each of R^{1b}, R^{1c}, and R^{1d} are C(═O)CH₃ or H. In certain embodiments, R^{1b} is the polysaccharide covalently conjugated to the surface protein, and each of R^{1a}, R^{1c}, and R^{1d} are C(═O)CH₃ or H. In certain embodiments, R^{1c} is the polysaccharide covalently conjugated to the surface protein, and each of R^{1a}, R^{1b}, and R^{1d} are C(═O)CH₃ or H. In certain embodiments, R^{1d} is the polysaccharide covalently conjugated to the surface protein, and each of R^{1a}, R^{1b}, and R^{1c} are C(═O)CH₃ or H. In certain embodiments, R^{1a} is the polysaccharide covalently conjugated to the surface protein, one of R^{1b}, R^{1c}, and R^{1d} is a monosaccharide or a terminal polysaccharide, and two of R^{1b}, R^{1c}, and R^{1d} are C(═O)CH₃ or H. In certain embodiments, R^{1b} is the polysaccharide covalently conjugated to the surface protein, one of R^{1a}, R^{1c}, and R^{1d} is a monosaccharide or a terminal polysaccharide, and two of R^{1a}, R^{1c}, and R^{1d} are C(═O)CH₃ or H. In certain embodiments, R^{1c} is the polysaccharide covalently conjugated to the surface protein, one of R^{1a}, R^{1b} and R^{1d} is a monosaccharide or a terminal polysaccharide, and two of R^{1a}, R^{1b}, and R^{1d} are C(═O)CH₃ or H. In certain embodiments, R^{1d} is the polysaccharide covalently conjugated to the surface protein, one of R^{1a}, R^{1b}, and R^{1c} is a monosaccharide or a terminal polysaccharide, and two of R^{1a}, R^{1b}, and R^{1c} are C(═O)CH₃ or H.

In certain embodiments, R³ is H.

In certain embodiments, each occurrence of R⁴ is H. In certain embodiments, two vicinal R⁴ substituents combine to form a fused-phenyl.

In certain embodiments, R⁵ is H.

In certain embodiments, R⁶ is H.

In certain embodiments, L¹ is —C(═O)CH₂—*.

In certain embodiments, L² is **—C(═O)(1,5-pentylene)C(═O)—.

In certain embodiments, Y is

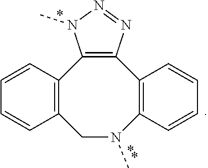

In certain embodiments, the optionally substituted heteroalkyl in Z is a polyethylene glycol (PEG) polymer. In certain embodiments, the optionally substituted heteroalkyl in Z is a polyamidoamine (PAMAM) polymer. In certain embodiments, the optionally substituted heteroalkyl in Z is a polyethyleneimine (PEI) polymer. In certain embodiments, the optionally substituted heteroalkyl in Z is a polymethyl methacrylate (PMMA) polymer. In certain embodiments, the optionally substituted heteroalkyl in Z is a poly(N-isopropyleneimine) (PPI) polymer. In certain embodiments, the optionally substituted heteroalkyl in Z is a polyvinyl alcohol (PVA) polymer.

In certain embodiments, the PEG polymer has the following formula:

—(CH₂CH₂O)ₒ—H,

wherein o is an integer ranging from 1 to 14,000.

In certain embodiments, the heteroalkyl or heteroalkylene in Z has a molecular weight of about 1 kDa. In certain embodiments, the heteroalkyl or heteroalkylene in Z has a molecular weight of about 5 kDa. In certain embodiments, the heteroalkyl or heteroalkylene in Z has a molecular weight of about 10 kDa. In certain embodiments, the heteroalkyl or heteroalkylene in Z has a molecular weight of about 100 kDa. In certain embodiments, the heteroalkyl or heteroalkylene in Z has a molecular weight of about 600 kDa.

In certain embodiments, R² is

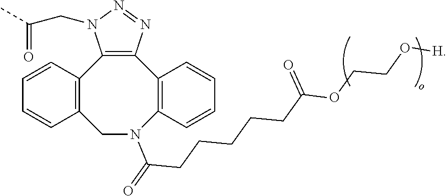

In certain embodiments, R² is

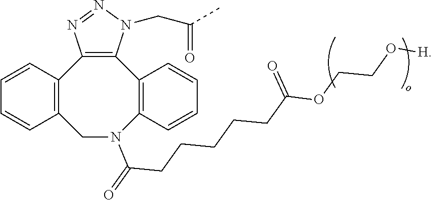

In certain embodiments, the modified immune cell or precursor thereof is a lymphocyte. In certain embodiments, the modified immune cell or precursor thereof is an immune effector cell. In certain embodiments, the modified immune cell or precursor thereof is selected from the group consisting of an αβ T cell, a γδ T cell, a CD8 T cell, a CD4 helper T cell, a CD4 regulatory T cell, an NK T cell, an NK cell, and any combination thereof. It is contemplated that the modified immune cell or precursor thereof may be derived from any immune cell that may capable of useful in vivo function when modified to express a chimeric antigen receptor.

Toxicity Related to Chimeric Antigen Therapy Chimeric antigen receptor T-cell immunotherapy has proven to be a very effective cancer immunotherapy. However, use of this treatment modality often induces life-threatening toxicities that can limit overall effectiveness. These toxicities can be broadly classified into two groups: autoimmune toxicities and cytokine-associated toxicities. Autoimmune toxicity, also called “on target, off-tumor toxicity,” results from antigen-specific attack on host tissues when the targeted antigen is expressed on nonmalignant tissue. Cytokine-associated toxicities are non-antigen-specific toxicities that occurs as a result of high-level immune activation. Examples of cytokine-associated toxicities include, but are not limited to, cytokine release syndrome (CRS), CAR-T cell-related encephalopathy syndrome (CRES), cytokine release encephalopathy syndrome, immune effector cell associated neurotoxicity syndrome (ICANS), and hemophagocytic lymphohistiocytosis/macrophage activation syndrome (HLH/MAS). In certain embodiments, a subject having a toxicity related to CAR treatment can have one or more of these toxicities.

The most common toxicity associated with CAR-based treatments is cytokine release syndrome (CRS). CRS is characterized by severe inflammatory symptoms caused by the activation of T cells and other immune cells resulting in the subsequent release of cytokines. Severe CRS usually develops within 24 h after CAR T cell infusion, with symptoms such as high fever, increased levels of acute-phase proteins and respiratory and cardiovascular insufficiency. If left untreated, this can lead to multiple organ dysfunction and death. CRS-related cytokines include IL-6, interferon gamma, IL-10, and IL-2 among others and may be produced by the CAR expressing T-cells directly or by other cells such as monocytes, macrophages, and dendritic cells in response to cytokines produced by the T-cells. Recent studies have demonstrated that host-derived monocyte and macrophage interactions with CAR-expressing T-cell interactions play a key role in the pathophysiology of CRS. In particular, CAR-T cells promoted recruitment and proliferation of monocytes by direct cell contact between CD40 (dendritic cells, monocytes, macrophages)-CD40 ligand (T-cell). which in-turn produced IL-1, IL-6 and nitric oxide (NO).

Neurotoxicity is the second most common toxicity related to CAR T cell therapy. Typical manifestations of neurotoxicity can appear from days to weeks after the disappearance of CRS-related symptoms and may escalate in severity to death. Symptoms range from minor headache and diminished attention to seizure, severe encephalopathy, and death. The most characteristic manifestation is encephalopathy typified by confusion progressing to expressive aphasia and at the extreme obtundation. Early signs are language and handwriting impairment followed by confusion, agitation, hallucinations, tremors and headaches. Seizures, motor weakness, incontinence, mental obtundation, increased intracranial pressure, papilloedema, and cerebral edema can be seen in severe cases of neurotoxicity.

Neurotoxicity and CRS often occur together with early confusion coinciding with high fevers and CRS, and later encephalopathy often following the resolution of CRS. The incidence and severity of neurotoxicity varies by different CAR constructs. Neurotoxicity may be more frequent in patients with pre-existing neurological conditions, younger patients and heavily pretreated patients. Notably, use of anti-IL-6 antibodies including tocilizumab, a front-line treatment for CRS, is often contraindicated in CAR-related neurotoxicity, in that anti-IL-6 antibodies have difficulty crossing the blood brain barrier and have not been found to have an effect on CAR T cell related encephalopathy. Further, some anti-IL-6 therapies can cause a temporary rise in IL-6 levels after the initial administration, which can exacerbate neurotoxicity symptoms.

### Polymer Labeling of Immune Cells

Recent studies have demonstrated that endogenous, host-derived monocytes and macrophages play key roles in mediating the development of CAR T cell-associate toxicities. Increased numbers of monocytes in humanized mice were associated with more severe CRS, and over-activation of monocytes and macrophages has been demonstrated to be a source of many toxic cytokines that induce CRS and neurotoxicity. For instance, upon activation, monocytes and macrophages secrete both IL-6 and IL-1, which, respectively, contribute to many of the key symptoms of CRS and lethal neurotoxicity during CAR T cell therapy. Further, CAR T cells do not induce CRS in monocyte-depleted mice, though antitumor efficacy was also attenuated. As such, monocytes are necessary for effective CAR T cell therapy. Direct contact between CAR T cells and monocytes/macrophages plays an important role in the activation of monocytes and macrophages. For example, the T cell surface proteins CD40 ligand, CD69, lymphocyte activation gene 3 (LAG3), and membrane-expressed TNF-α have been shown to activate monocytes and macrophages through mechanisms involving cell-to-cell contact.

As such, decreasing monocyte over-activation by controlling cell-to-cell interactions between CAR T cells and monocytes provides a solution for the management of toxicities including CRS and neurotoxicity. Thus, in certain embodiments of the present disclosure, the modified immune cells or precursor cells of the present invention comprise spacer molecules conjugated to the surface of the cells. In certain embodiments, these spacer molecules comprise polyethylene glycol (PEG) molecules such that the modified immune cells become “pegylated” or labeled with surface-bound PEG molecules. These pegylated cells can reduce or prevent CRS-related symptoms and neurotoxicity by forming a polymeric spacer around the modified immune cell, which greatly decreases cell-to-cell interactions between CAR T cells and monocytes by steric hindrance, and thus reduces or prevents over-activation of monocytes by CAR T cells. Over time, the slow expansion of CAR T cells reduces the surface density of PEG surface density and restores CAR T cell-tumor cell interactions to induce potent tumor killing. This restoration occurs prior to the return of CAR T cell-monocyte interactions, which enables CAR T cells to kill tumor cells with only limited monocyte activation. It is also contemplated that other types of large, biologically inert molecules could be used as spacer molecules which physically inhibit cell-cell interactions, including but not limited to alginates, microparticles, dendrimers, other polymers, and DBCO-modified red blood cells.

To enable in situ or ex vivo labeling (e.g. by pegylation), the surface of CAR T cells were first metabolically labeled with azide groups before infusion into the subject. This metabolic labeling process is accomplished by culturing the T cells or immune cells in the presence of an azido glycan (e.g. N-azidoacetylmannosamine-tetraacylated, Ac4ManNAz). The metabolically active cells take up the azido glycan and incorporate it into the plasma membrane. In certain embodiments, the labeling process can occur prior to, subsequent to, or concurrent with transfection or transduction of the cells with constructs encoding the CAR.

In certain embodiments, dibenzocyclooctyne (DBCO)-conjugated spacer molecules (e.g. DBCO-PEG) are administered following labeling with azido glycan. The DBCO-spacer molecules then become conjugated to the CAR T cell surface via DBCO-azide click chemistry to form a polymeric spacer around the CAR T cells. In certain embodiments, the administration of the DBCO-spacer molecules occurs ex vivo following azido glycan labeling and prior to infusion into the subject. In certain embodiments, the administration of the DBCO-spacer occurs after the azido glycan labeled CAR T cells have been administered to the subject such that the conjugation occurs in situ. The conjugation of spacer molecules (e.g. PEG) to the cell surface greatly decreases cell-to-cell interactions between CAR T cells, tumor cells, and monocytes via, for example, steric hindrance, and thus abolishes the over-activation of monocytes by CAR T cells and prevents or reduces the severity of toxicities that result from interactions with endogenous immune cells (e.g. CRS and/or neurotoxicity). In certain embodiments, the subsequent slow expansion of CAR T cells reduces the surface density of spacer molecules and restores CAR T cell-tumor cell interactions to induce potent tumor killing. This restoration of cytotoxic function occurs prior to the return of CAR T cell-monocyte interactions, which enables CAR T cells to kill tumor cells with limited monocyte activation.

In certain embodiments, the invention of the present disclosures includes modified immune cells or precursor cells thereof comprising chimeric antigen receptors and surface-conjugated spacer molecules (e.g. PEG). It is contemplated that the modified immune cells of the invention can comprise any immune cell useful for CAR-therapy, wherein the CAR therapy has the chance of causing an associated toxicity (e.g. CRS and/or neurotoxicity). Non-limiting examples of immune cells commonly used in CAR therapy include αβ T cells, γδ T cells, CD8⁺ T cells, CD4⁺ helper T cells, CD4⁺ regulatory T cell, NK T cells, NK cells, or combinations thereof. The skilled artisan would understand that a particular immune cell or precursor cell can be selected based on the desired CAR construct and treatment application.

### Methods

In one aspect, the present disclosure provides a method of preparing the modified immune cell or precursor cell thereof comprising a chimeric antigen receptor and comprising the polysaccharide of formula (I), the method comprising:
(a) contacting a modified immune cell comprising a CAR and one or more surface glycans, wherein at least one of the surface glycans comprises a polysaccharide of formula (II):

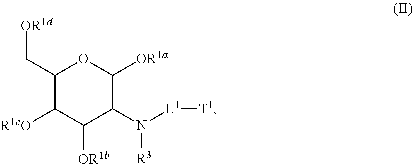

wherein:
R^{1a}, R^{1b}, R^{1c}, and R^{1d} are each independently selected from the group consisting of H, optionally substituted C₁-C₆ alkyl, optionally substituted C₂-C₆ heteroalkyl, optionally substituted C₃-C₈ cycloalkyl, optionally substituted phenyl, optionally substituted benzyl, optionally substituted C₂-C₉ heterocyclyl, C(═O)OR^{a}, C(═O)R^{a}, C(═O)N(R^{a})(R^{b}), S(═O)₂R^{a}, S(═O)₂N(R^{a})(R^{b}), a monosaccharide, a terminal polysaccharide, and a polysaccharide covalently conjugated to a CAR-T cell surface protein,
   wherein one selected from R^{1a}, R^{1b}, R^{1c}, and R^{1d} is the polysaccharide covalently conjugated to the surface protein;
R³ is selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
R⁴ is selected from the group consisting of optionally substituted C₁-C₆ alkyl, C₂-C₆ heteroalkyl, optionally substituted C₃-C₈ cycloalkyl, optionally substituted phenyl, optionally substituted benzyl, optionally substituted C₂-C₉ heterocyclyl, halogen, OR^{a}, N(R^{a})(R^{b}), SR^{a}, CN, and NO₂,
   wherein two adjacent R⁴ substituents may combine with the atoms to which they are bound to form an optionally substituted phenyl, optionally substituted C₃-C₈ cycloalkyl, or optionally substituted C₂-C₉ heterocyclyl;
R⁵ is selected from the group consisting of H and optionally substituted C₁-C₆ alkyl;
R⁶ is selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
L¹ is selected from the group consisting of —C(═O)(optionally substituted C₁-C₆ alkylene)-*, —C(═O)(optionally substituted C₁-C₆ heteroalkylene)-*, -(optionally substituted C₁-C₆ alkylene)-*, and -(optionally substituted C₁-C₆ heteroalkylene)-*;
T¹ is selected from the group consisting of N₃,

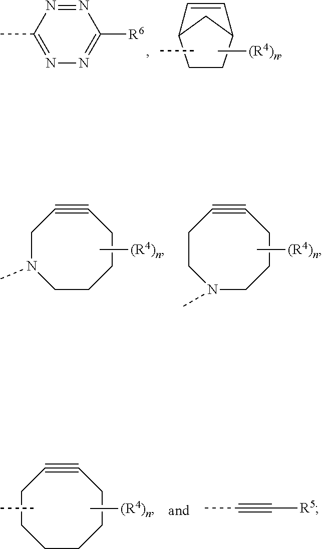

each occurrence of R^{a} and R^{b} is independently selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
n is an integer ranging from 0 to 11; and
(b) a compound of formula (III):

T²-L²-Z (III),

wherein:
T² is selected from the group consisting of —N₃,

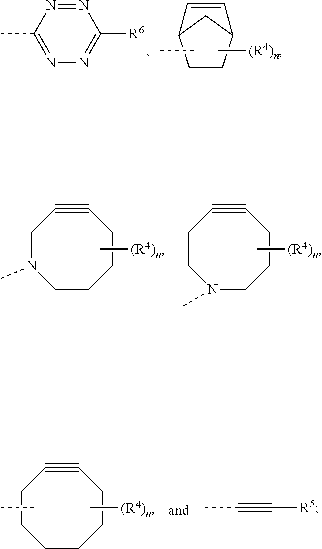

and
L² is selected from the group consisting of **—C(═O)(optionally substituted C₁-C₁₂ alkylene)C(═O)O—, **—C(═O)(optionally substituted C₂-C₁₂ heteroalkylene)C(═O)O—, **—C(═O)(optionally substituted C₁-C₁₂ alkylene)O—, **—C(═O)(optionally substituted C₂-C₁₂ heteroalkylene)O—, **-(optionally substituted C₁-C₁₂ alkylene)C(═O)O—, **-(optionally substituted C₂-C₁₂ heteroalkylene)C(═O)O—, **-(optionally substituted C₁-C₁₂ alkylene)O—, and **-(optionally substituted C₂-C₁₂ heteroalkylene)C(═O)O—;
Z is selected from the group consisting of an optionally substituted heteroalkyl group, a dendrimer, a microparticle, a nanoparticle, an alignate, a biomolecule, and a modified red blood cell,
   wherein the dendrimer, microparticle, nanoparticle, alignate, biomolecule, or modified red blood cell can be covalently linked to L² by an optionally substituted heteroalkylene, and
   wherein each heteroalkyl or heteroalkylene has a molecular weight ranging from about 1 kDa to about 600 kDa.
      In certain embodiments, T¹ is selected from the group consisting of —N₃ and

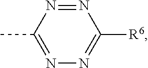

and T² is selected from the group consisting of

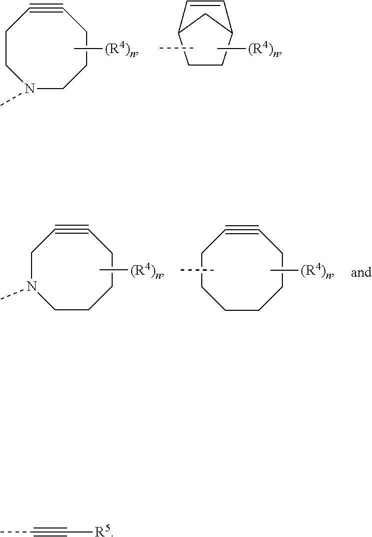

In certain embodiments, T¹ is selected from the group consisting of

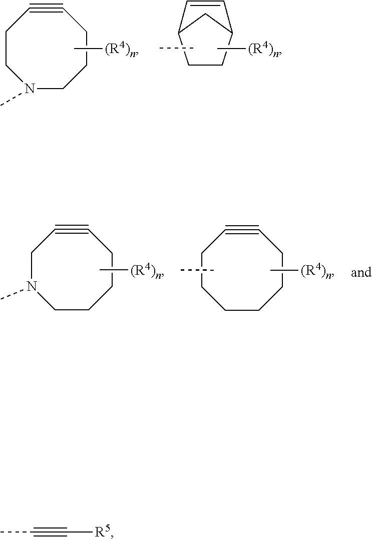

and T² is selected from the group consisting of —N₃ and

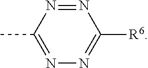

In certain embodiments, each occurrence of optionally substituted alkyl, optionally substituted heteroalkyl, optionally substituted cycloalkyl, optionally substituted alkoxy, optionally substituted phenyl, optionally substituted benzyl, optionally substituted heterocyclyl, optionally substituted alkylene, and optionally substituted heteroalkylene, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₉ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

In certain embodiments, the compound of formula (II) is a compound of formula (IIa):

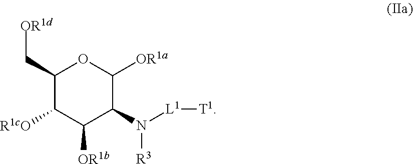

In certain embodiments, R^{1a} is the polysaccharide covalently conjugated to the surface protein, and each of R^{1b}, R^{1c}, and R^{1d} are C(═O)CH₃ or H. In certain embodiments, R^{1b} is the polysaccharide covalently conjugated to the surface protein, and each of R^{1a}, R^{1b}, and R^{1d} are C(═O)CH₃ or H. In certain embodiments, R^{1c} is the polysaccharide covalently conjugated to the surface protein, and each of R^{1a}, R^{1b}, and R^{1d} are C(═O)CH₃ or H. In certain embodiments, R^{1d} is the polysaccharide covalently conjugated to the surface protein, and each of R^{1a}, R^{1b}, and R^{1c} are C(═O)CH₃ or H. In certain embodiments, R^{1a} is the polysaccharide covalently conjugated to the surface protein, one of R^{1b}, R^{1c}, and R^{1d} is a monosaccharide or a terminal polysaccharide, and two of R^{1b}, R^{1c}, and R^{1d} are C(═O)CH₃ or H. In certain embodiments, R^{1b} is the polysaccharide covalently conjugated to the surface protein, one of R^{1a}, R^{1c}, and R^{1d} is a monosaccharide or a terminal polysaccharide, and two of R^{1a}, R^{1c}, and R^{1d} are C(═O)CH₃ or H. In certain embodiments, R^{1c} is the polysaccharide covalently conjugated to the surface protein, one of R^{1a}, R^{1b}, and R^{1d} is a monosaccharide or a terminal polysaccharide, and two of R^{1a}, R^{1b}, and R^{1d} are C(═O)CH₃ or H. In certain embodiments, R^{1d} is the polysaccharide covalently conjugated to the surface protein, one of R^{1a}, R^{1b}, and Ric is a monosaccharide or a terminal polysaccharide, and two of R^{1a}, R^{1b}, and R^{1c} are C(═O)CH₃ or H.

In certain embodiments, R³ is H.

In certain embodiments, each occurrence of R⁴ is H. In certain embodiments, two vicinal R⁴ substituents combine to form a fused-phenyl.

In certain embodiments, R⁵ is H.

In certain embodiments, R⁶ is H.

In certain embodiments, L¹ is —C(═O)CH₂—*.

In certain embodiments, L² is **—C(═O)(1,5-pentylene)C(═O)—.

In certain embodiments, the optionally substituted heteroalkyl in Z is a polyethylene glycol (PEG) polymer. In certain embodiments, the optionally substituted heteroalkyl in Z is a polyamidoamine (PAMAM) polymer. In certain embodiments, the optionally substituted heteroalkyl in Z is a polyethyleneimine (PEI) polymer. In certain embodiments, the optionally substituted heteroalkyl in Z is a polymethyl methacrylate (PMMA) polymer. In certain embodiments, the optionally substituted heteroalkyl in Z is a poly(N-isopropyleneimine) (PPI) polymer. In certain embodiments, the optionally substituted heteroalkyl in Z is a polyvinyl alcohol (PVA) polymer.

In certain embodiments, the PEG polymer has the following formula:

—(CH₂CH₂O)ₒ—H,
wherein o is an integer ranging from 1 to 14,000.

In certain embodiments, the heteroalkyl or heteroalkylene in Z has a molecular weight of about 1 kDa. In certain embodiments, the heteroalkyl or heteroalkylene in Z has a molecular weight of about 5 kDa. In certain embodiments, the heteroalkyl or heteroalkylene in Z has a molecular weight of about 10 kDa. In certain embodiments, the heteroalkyl or heteroalkylene in Z has a molecular weight of about 100 kDa. In certain embodiments, the heteroalkyl or heteroalkylene in Z has a molecular weight of about 600 kDa.

In certain embodiments, T¹ is N₃.

In certain embodiments, T² is

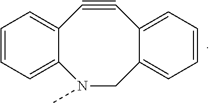

In certain embodiments, the compound of formula (III) is:

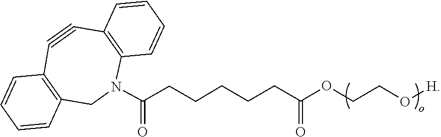

In another aspect, the invention provides a method for reducing the severity of a toxicity in a subject caused by administration of a chimeric antigen receptor (CAR) expressing T cell to the subject, said method comprising:
a. labeling the CAR expressing T cell with an azido glycan and/or a trans-cyclooctene group and/or a tetrazine group, and
b. conjugating a spacer molecule to the azido glycan and/or trans-cyclooctene group and/or tetrazine group thereby producing conjugated CAR T cell; wherein the PEGylation reduces the interaction of the CAR T cell with endogenous immune cells, thereby reducing the severity of the toxicity. In certain embodiments, the labeling of the CAR T cell with an azido glycan and/or trans-cyclooctene group and/or tetrazine group occurs ex vivo from the subject. In certain embodiments, the labeling of the cells with azido glycan occurs by culturing the cells in media supplemented with an azido glycan (e.g. N-azidoacetylmannosamine-tetraacylated, Ac4ManNAz) such that the cells take up and incorporate the azido glycan into the plasma membrane.

In certain embodiments, the CAR expressing T cell is labeled with an azido glycan and the spacer molecule comprises a dibenzocyclooctyne (DBCO) group.

In certain embodiments, the CAR expressing T cell is labeled with a trans-cyclooctene group and the spacer molecule comprises a tetrazine group.

In certain embodiments, the CAR expressing T cell is labeled with a tetrazine group and the spacer molecule comprises a trans-cyclooctene group.

In certain embodiments, the spacer molecule is a compound of formula (III).

In certain embodiments, an effective amount of the spacer molecule is administered to the subject such that the conjugation of the spacer molecule occurs in vivo in the subject. In certain embodiments, the spacer comprises a PEG linker. In certain embodiments, PEG is a polyether compound with a formula commonly expressed as H—(O−CH2-CH2)n-OH, where n represents a carbon chain of varying lengths. PEG compounds are typically identified by an number corresponding to their molecular weight in daltons. It is contemplated that any PEG or PEG derivative can be used in the invention, including but not limited to monomer PEGs, branched PEGs, star PEGs, and comb PEGs. PEGs are typically synthesized by the polymerization of ethylene oxide and are commonly available over a wide range of molecular weights ranging from 300 g/mol to 10,000,000 g g/mol. In certain embodiments, the PEG is PEG 1 k, PEG 5 k, PEG 10 k, PEG 100 k, PEG 600 k, and/or any combination thereof. The skilled artisan would be able to select a PEG molecule and weight appropriate to inhibit or reduce a particular CAR treatment-related toxicity. In certain preferred embodiments of the current invention, the PEG is PEG 600 k.

In certain embodiments, the conjugation of the spacer molecule occurs after administering the CART cell to the subject. In certain embodiments, the conjugation of the spacer molecule occurs prior to administration to the subject. In certain embodiments, the toxicity is selected from the group consisting of cytokine release syndrome (CRS), CAR-T cell-related encephalopathy syndrome (CRES), cytokine release encephalopathy syndrome, immune effector cell associated neurotoxicity syndrome (ICANS), hemophagocytic lymphohistiocytosis/macrophage activation syndrome (HLH/MAS), and any combination thereof. In certain embodiments, the effective amount of spacer molecule is administered to the subject when the subject presents at least one symptom associated with the toxicity. Non-limiting examples of CAR-associated toxicities can include, but are not limited to fever, rigors, elevated IL-6 levels, elevated IL-5 levels, elevated IL-13 levels, elevated IL-10 levels, elevated interferon-gamma (IFNγ) levels dermatitis, tachycardia, hypotension, headache, nausea, aphasia, disorientation, lethargy, and any combination thereof.

### Methods of Generating Modified Immune Cells

Any modified cell comprising a CAR comprising any antigen binding domain, any hinge, any transmembrane domain, any intracellular costimulatory domain, and any intracellular signaling domain described herein is envisioned, and can readily be understood and used a person of skill in the art to generate the modified immune cells of the invention in view of the disclosure herein.

In some embodiments, the modified cell is an immune cell or precursor cell thereof. In an exemplary embodiment, the modified cell is a T cell. In an exemplary embodiment, the modified cell is an autologous cell. In an exemplary embodiment, the modified cell is an autologous immune cell or precursor cell thereof. In an exemplary embodiment, the modified cell is an autologous T cell.

The present invention provides methods for producing/generating a modified immune cell or precursor cell thereof (e.g., a T cell/NK cell/NKT cell). The cells are generally engineered by introducing a nucleic acid encoding a subject CAR and metabolic labeling the plasma membrane of the cells with an azido glycan (e.g. N-azidoacetylmannosamine-tetraacylated, Ac4ManNAz).

Methods of metabolically labeling the cells with an azido glycan (e.g. N-azidoacetylmannosamine-tetraacylated, Ac4ManNAz) include, but are not limited to feeding the azido glycan to dividing, metabolically active cells such that the azido glycan are integrated by the glycan biosynthetic machinery into various glycoconjugates throughout the cell, including the plasma membrane. Azido glycans can be provided to the cells directly in culture media or by packaging the azido glycans into liposomes or lipid-based nanoparticles. In the context of CAR T cells, this process can occur while allogeneic or autologous immune cells are being stimulated ex vivo for introduction of the nucleic acid construct or vector encoding the CAR receptor into the cells.

Methods of introducing nucleic acids into a cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, MA) or the Gene Pulser II (BioRad, Denver, CO), Multiporator (Eppendorf, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

In some embodiments, a nucleic acid encoding a subject CAR of the invention is introduced into a cell by an expression vector. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggyback, and Integrases such as Phi31. Some other suitable expression vectors include herpes simplex virus (HSV) and retrovirus expression vectors.

Adenovirus expression vectors are based on adenoviruses, which have a low capacity for integration into genomic DNA but a high efficiency for transfecting host cells. Adenovirus expression vectors contain adenovirus sequences sufficient to: (a) support packaging of the expression vector and (b) to ultimately express the subject CAR in the host cell. In some embodiments, the adenovirus genome is a 36 kb, linear, double stranded DNA, where a foreign DNA sequence (e.g., a nucleic acid encoding a subject CAR) may be inserted to substitute large pieces of adenoviral DNA in order to make the expression vector of the present invention (see, e.g., Danthinne and Imperiale, Gene Therapy (2000) 7(20): 1707-1714).

Another expression vector is based on an adeno associated virus, which takes advantage of the adenovirus coupled systems. This AAV expression vector has a high frequency of integration into the host genome. It can infect non-dividing cells, thus making it useful for delivery of genes into mammalian cells, for example, in tissue cultures or in vivo. The AAV vector has a broad host range for infectivity. Details concerning the generation and use of AAV vectors are described in U.S. Pat. Nos. 5,139,941 and 4,797,368.

Retrovirus expression vectors are capable of integrating into the host genome, delivering a large amount of foreign genetic material, infecting a broad spectrum of species and cell types and being packaged in special cell lines. The retrovirus vector is constructed by inserting a nucleic acid (e.g., a nucleic acid encoding a subject CAR) into the viral genome at certain locations to produce a virus that is replication defective. Though the retrovirus vectors are able to infect a broad variety of cell types, integration and stable expression of the subject CAR, requires the division of host cells.

Lentivirus vectors are derived from lentiviruses, which are complex retroviruses that, in addition to the common retroviral genes gag, pol, and env, contain other genes with regulatory or structural function (see, e.g., U.S. Pat. Nos. 6,013,516 and 5,994,136). Some examples of lentiviruses include the human immunodeficiency viruses (HIV-1, HIV-2) and the simian immunodeficiency virus (SIV). Lentivirus vectors have been generated by multiply attenuating the HIV virulence genes, for example, the genes env, vif, vpr, vpu and nef are deleted making the vector biologically safe. Lentivirus vectors are capable of infecting non-dividing cells and can be used for both in vivo and ex vivo gene transfer and expression, e.g., of a nucleic acid encoding a subject CAR (see, e.g., U.S. Pat. No. 5,994,136).

Expression vectors including a nucleic acid of the present disclosure can be introduced into a host cell by any means known to persons skilled in the art. The expression vectors may include viral sequences for transfection, if desired. Alternatively, the expression vectors may be introduced by fusion, electroporation, biolistics, transfection, lipofection, or the like. The host cell may be grown and expanded in culture before introduction of the expression vectors, followed by the appropriate treatment for introduction and integration of the vectors. The host cells are then expanded and may be screened by virtue of a marker present in the vectors. Various markers that may be used are known in the art, and may include hprt, neomycin resistance, thymidine kinase, hygromycin resistance, etc. As used herein, the terms “cell,” “cell line,” and “cell culture” may be used interchangeably. In some embodiments, the host cell is an immune cell or precursor thereof, e.g., a T cell, an NK cell, or an NKT cell.

The present invention also provides genetically engineered or modified immune cells which include and stably express a CAR. In some embodiments, the genetically engineered or modified cells are genetically engineered or modified T-lymphocytes (T cells), regulatory T cells (Tregs), naive T cells (TN), memory T cells (for example, central memory T cells (TCM), effector memory cells (TEM)), natural killer cells (NK cells), natural killer T cells (NKT cells) and macrophages capable of giving rise to therapeutically relevant progeny. In one embodiment, the genetically engineered or modified cells are autologous cells.

Modified cells (e.g., comprising a subject CAR) may be produced by stably transfecting or transducing host cells with an expression vector including a nucleic acid encoding a CAR. Additional methods to generate a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). Transfected cells expressing a subject CAR of the present disclosure may be expanded ex vivo.

Physical methods for introducing an expression vector into host cells include calcium phosphate precipitation, lipofection, particle bombardment, microinjection, electroporation, and the like. Methods for producing cells including vectors and/or exogenous nucleic acids are well-known in the art. See, e.g., Sambrook et al. (2001), Molecular Cloning: A Laboratory Manual, Cold Spring Harbor Laboratory, New York.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20 °C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids encoding CARs into a host cell, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Moreover, the nucleic acids may be introduced by any means. such as transducing the expanded T cells, transfecting the expanded T cells, and electroporating the expanded T cells. One nucleic acid may be introduced by one method and another nucleic acid may be introduced into the T cell by a different method.

### Sources of Immune Cells

Prior to expansion, a source of immune cells is obtained from a subject for ex vivo manipulation. Sources of target cells for ex vivo manipulation may also include, e.g., autologous or heterologous donor blood, cord blood, or bone marrow. For example, the source of immune cells may be from the subject to be treated with the modified immune cells of the invention, e.g., the subject's blood, the subject's cord blood, or the subject's bone marrow. Non-limiting examples of subjects include humans, dogs, cats, mice, rats, and transgenic species thereof. In certain exemplary embodiments, the subject is a human.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells and/or NKT cells. Other exemplary cells include stem cells, such as multipotent and pluripotent stem cells, including induced pluripotent stem cells (iPSCs). In certain aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8⁺ T cell (e.g., a CD8⁺ naive T cell, central memory T cell, or effector memory T cell), a CD4⁺ T cell, a natural killer T cell (NKT cells), a regulatory T cell (Treg), a stem cell memory T cell, a lymphoid progenitor cell, a hematopoietic stem cell, a natural killer cell (NK cell), a natural killer T cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8⁺ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells. mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In certain aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity-based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in certain aspects, contain lymphocytes, including T cells, monocytes, granulocytes, B cells, other nucleated white blood cells, red blood cells, and/or platelets, and in certain aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, a washing step is accomplished by tangential flow filtration (TFF) according to the manufacturer's instructions. In certain embodiments, the cells are resuspended in a variety of biocompatible buffers after washing. In certain embodiments, components of a blood cell sample are removed, and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune cells are obtained from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or another saline solution with or without buffer. In some embodiments, the undesirable components of the apheresis sample may be removed, and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in certain aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. Such separation steps can be based on positive selection, in which the cells having bound the reagents are retained for further use, and/or negative selection, in which the cells having not bound to the antibody or binding partner are retained. In some examples, both fractions are retained for further use. In certain aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In certain exemplary embodiments, multiple rounds of separation steps are carried out, where the positively or negatively selected fraction from one step is subjected to another separation step, such as a subsequent positive or negative selection. In certain exemplary embodiments, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker) or express relatively low levels (marker^{low}) of one or more markers. For example, in certain aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8⁺ cells or the T cells, e.g., CD3⁺ cells) are enriched for (i.e., positively selected for) cells that are positive or expressing high surface levels of CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD127). In certain exemplary embodiments, CD8+ T cells are enriched for cells positive for CD45RO (or negative for CD45RA) and for CD62L. For example, CD3⁺, CD28⁺ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD14. In certain aspects, a CD4⁺ or CD8⁺ selection step is used to separate CD4⁺ helper and CD8+ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations. In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in certain aspects is particularly robust in such sub-populations. In some embodiments, combining TCM-enriched CD8⁺ T cells and CD4⁺ T cells further enhances efficacy.

In some embodiments, memory T cells are present in both CD62L⁺ and CD62L⁻ subsets of CD8⁺ peripheral blood lymphocytes. PBMC can be enriched for or depleted of CD62L⁻CD8⁺ and/or CD62L⁺CD8⁺ fractions, such as using anti-CD8 and anti-CD62L antibodies. In some embodiments, a CD4⁺ T cell population and/or a CD8⁺ T population is enriched for central memory (TCM) cells. In some embodiments, the enrichment for central memory T (TCM) cells is based on positive or high surface expression of CD45RO, CD62L. CCR7, CD28, CD3, and/or CD127; in certain aspects, it is based on negative selection for cells expressing or highly expressing CD45RA and/or granzyme B. In certain aspects, isolation of a CD8⁺ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in certain aspects are carried out simultaneously and in other aspects are carried out sequentially, in either order. In some embodiments, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

CD4⁺ T helper cells are sorted into naive, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L⁻ and CD45RO. In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In certain aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. An exemplary method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80 °C at a rate of 1 °C per minute and stored in the vapor phase of a liquid nitrogen storage tank. Other methods of controlled freezing may be used as well as uncontrolled freezing immediately at −20 °C or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

### Expansion of Immune Cells

Whether prior to or after modification of cells to express a subject CAR, the cells can be activated and expanded in number using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Publication No. 20060121005. For example, the immune cells of the invention may be expanded by contact with a surface having attached thereto an agent that stimulates a CD3/TCR complex associated signal and a ligand that stimulates a co-stimulatory molecule on the surface of the immune cells. In particular, immune cell populations may be stimulated by contact with an anti-CD3 antibody, or an antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the immune cells, a ligand that binds the accessory molecule is used. For example, immune cells can be contacted with an anti-CD3 antibody and an anti-CD28 antibody, under conditions appropriate for stimulating proliferation of the immune cells. Examples of an anti-CD28 antibody include 9.3, B-T3, XR-CD28 (Diaclone, Besancon, France) and these can be used in the invention, as can other methods and reagents known in the art (see, e.g., ten Berge et al., Transplant Proc. (1998) 30(8): 3975-3977; Haanen et al., J. Exp. Med. (1999) 190(9): 1319-1328; and Garland et al., J. Immunol. Methods (1999) 227(1-2): 53-63).

Expanding the immune cells by the methods disclosed herein can be multiplied by about 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700 fold, 800-fold, 900-fold, 1000-fold, 2000-fold, 3000-fold, 4000-fold, 5000-fold, 6000-fold, 7000-fold, 8000-fold, 9000-fold, 10,000-fold, 100,000-fold, 1,000,000-fold, 10,000,000-fold, or greater, and any and all whole or partial integers therebetween. In one embodiment, the immune cells expand in the range of about 20-fold to about 50-fold.

Following culturing, the immune cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. In certain exemplary embodiments, the level of confluence is 70% or greater before passing the cells to another culture apparatus. In particularly exemplary embodiments, the level of confluence is 90% or greater. A period of time can be any time suitable for the culture of cells in vitro. The immune cell medium may be replaced during the culture of the immune cells at any time. In certain exemplary embodiments, the immune cell medium is replaced about every 2 to 3 days. The immune cells are then harvested from the culture apparatus whereupon the immune cells can be used immediately or cryopreserved to be stored for use at a later time. In one embodiment, the invention includes cryopreserving the expanded immune cells. The cryopreserved immune cells are thawed prior to introducing nucleic acids into the immune cell.

In another embodiment, the method comprises isolating immune cells and expanding the immune cells. In another embodiment, the invention further comprises cryopreserving the immune cells prior to expansion. In yet another embodiment, the cryopreserved immune cells are thawed for electroporation with the RNA encoding the chimeric membrane protein.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of immune cells comprises the addition to the cellular growth factors, such as those described in U.S. Pat. No. 5,199,942, or other factors, such as FLT3-L, IL-1, IL-3 and c-kit ligand. In one embodiment, expanding the immune cells comprises culturing the immune cells with a factor selected from the group consisting of FLT3-L, IL-1, IL-3 and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging. Therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for immune cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of immune cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37 °C) and atmosphere (e.g., air plus 5% CO₂).

The medium used to culture the immune cells may include an agent that can co-stimulate the immune cells. For example, an agent that can stimulate CD3 is an antibody to CD3, and an agent that can stimulate CD28 is an antibody to CD28. This is because, as demonstrated by the data disclosed herein, a cell isolated by the methods disclosed herein can be expanded approximately 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10,000-fold, 100,000-fold, 1,000,000-fold, 10,000,000-fold, or greater. In one embodiment, the immune cells expand in the range of about 2-fold to about 50-fold, or more by culturing the electroporated population. In one embodiment, human T regulatory cells are expanded via anti-CD3 antibody coated KT64.86 artificial antigen presenting cells (aAPCs). Methods for expanding and activating immune cells can be found in U.S. Pat. Nos. 7,754,482, 8,722,400, and 9,555,105, the contents of which are incorporated herein in their entirety.

In one embodiment, the method of expanding the immune cells can further comprise isolating the expanded immune cells for further applications. In another embodiment, the method of expanding can further comprise a subsequent electroporation of the expanded immune cells followed by culturing. The subsequent electroporation may include introducing a nucleic acid encoding an agent, such as a transducing the expanded immune cells, transfecting the expanded immune cells, or electroporating the expanded immune cells with a nucleic acid, into the expanded population of immune cells, wherein the agent further stimulates the immune cell. The agent may stimulate the immune cells, such as by stimulating further expansion, effector function, or another immune cell function.

Methods of Treatment, Amelioration, and/or Prevention

The modified immune cells or precursor cells (e.g., T cells) described herein may be included in a composition for immunotherapy. The composition may include a pharmaceutical composition and further include a pharmaceutically acceptable carrier. A therapeutically effective amount of the pharmaceutical composition comprising the modified immune cells may be administered.

In one aspect, the invention includes a method for reducing the severity of a toxicity in a subject caused by administration of a chimeric antigen receptor (CAR) expressing T cell to the subject, said method comprising labeling the CAR expressing T cell with an azido glycan (e.g. ManNAz), and conjugating a spacer molecule to the azido glycan label thereby producing a conjugated CAR T cell; wherein the conjugation reduces the interaction of the CAR T cell with endogenous immune cells, thereby reducing the severity of the toxicity. In certain embodiments, the labeling of the CAR T cell with an azido glycan occurs ex vivo from the subject. In certain embodiments, the effective amount of spacer molecule is administered to the subject such that the conjugation of the spacer molecule occurs in vivo in the subject. In certain embodiments, the conjugation of the spacer molecule occurs after administering the CAR T cell to the subject. In certain embodiments, the spacer molecule administration occurs after the administration of the CAR T cell to the subject. In certain embodiments, the azido glycan is N-azidoacetylmannosamine-tetraacylated (Ac4ManNAz). In certain embodiments, the PEG is PEG 600 k. In certain embodiments, the spacer molecule is a compound of formula (III).

In certain embodiments, the toxicity is selected from the group consisting of cytokine release syndrome (CRS), CAR-T cell-related encephalopathy syndrome (CRES), cytokine release encephalopathy syndrome, immune effector cell associated neurotoxicity syndrome (ICANS), hemophagocytic lymphohistiocytosis/macrophage activation syndrome (HLH/MAS), and any combination thereof. In certain embodiments, the effective amount of spacer molecule is administered to the subject when the subject presents at least one symptom associated with the toxicity. In certain embodiments, the symptom associated with the toxicity is selected from the group consisting of fever, rigors, elevated IL-6 levels, elevated IL-5 levels, elevated IL-13 levels, elevated IL-10 levels, elevated interferon-gamma (IFNγ) levels dermatitis, tachycardia, hypotension, headache, nausea, aphasia, disorientation, lethargy, and any combination thereof. Clinical methods of monitoring the subject of the symptoms associated with CAR treatment toxicity are well known in the art.

Also included is a method of treating, ameliorating, and/or preventing a disease or condition in a subject in need thereof comprising administering to the subject a modified immune cell (e.g., a modified CAR T cell) or precursor thereof wherein the immune cell comprises a chimeric antigen receptor (CAR) specific for a disease-related antigen and comprising an azido glycan group and/or trans-cyclooctene group and/or tetrazine group. In certain embodiments, the method further comprises administering a composition comprising an effective amount of a spacer molecule when the patient experiences a toxicity related to the administration of the modified immune cell such that the spacer molecule is conjugated to the azido glycan groups on the surface of the cell. In certain embodiments, the CAR expressing T cell is labeled with an azido glycan and the spacer molecule comprises a dibenzocyclooctyne (DBCO) group. In certain embodiments, the CAR expressing T cell is labeled with a trans-cyclooctene group and the spacer molecule comprises a tetrazine group. In certain embodiments, the CAR expressing T cell is labeled with a tetrazine group and the spacer molecule comprises a trans-cyclooctene group.

In certain embodiments, administration of the spacer molecule reduces the interaction of the modified immune cells with endogenous immune cells such that the toxicity is resolved or reduced in severity. In certain embodiments, the toxicity is selected from the group consisting of cytokine release syndrome (CRS), CAR-T cell-related encephalopathy syndrome (CRES), cytokine release encephalopathy syndrome, immune effector cell associated neurotoxicity syndrome (ICANS), hemophagocytic lymphohistiocytosis/macrophage activation syndrome (HLH/MAS), and any combination thereof. In certain embodiments, the effective amount of spacer molecule is administered to the subject when the subject presents at least one symptom associated with the toxicity. In certain embodiments, the symptom associated with the toxicity is selected from the group consisting of fever, rigors, elevated IL-6 levels, elevated IL-5 levels, elevated IL-13 levels, elevated IL-10 levels, elevated interferon-gamma (IFNγ) levels dermatitis, tachycardia, hypotension, headache, nausea, aphasia, disorientation, lethargy, and any combination thereof. Clinical methods of monitoring the subject of the symptoms associated with CAR treatment toxicity are well known in the art. In certain embodiments, the disease is a cancer. In certain embodiments, the spacer molecule is a compound of formula (TTT).

Methods for administration of modified immune cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338. In some embodiments, the cell therapy, e.g., adoptive T cell therapy is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease, e.g. the cancer, prior to administration of the modified immune cells or composition comprising the modified immune cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In certain embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse. In some aspects, the subject has not received prior treatment with another therapeutic agent.

In certain embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

The modified immune cells and precursor cells of the present invention can be administered to an animal, preferably a mammal, even more preferably a human, to treat, ameliorate, and/or prevent a cancer. In addition, the cells of the present invention can be used for the treatment of any condition related to a cancer, especially a cell-mediated immune response against a tumor cell(s), where it is desirable to treat or alleviate the disease. The types of cancers to be treated with the modified cells or pharmaceutical compositions of the invention include certain solid tumors including metastatic cancers. Adult tumors/cancers and pediatric tumors/cancers are also included.

The cells of the invention to be administered may be autologous, with respect to the subject undergoing therapy.

The administration of the cells of the invention may be carried out in any convenient manner known to those of skill in the art. The cells of the present invention may be administered to a subject by aerosol inhalation, injection, ingestion, transfusion, implantation or transplantation. Likewise, the administration of the compositions comprising the spacer molecule may be carried out in any convenient manner known to those of skill in the art. The compositions may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention and/or compositions of the invention are injected directly into a site of inflammation in the subject, a local disease site in the subject, a lymph node, an organ, a tumor, and the like.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4⁺ to CD8⁺ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 1×10⁵ cells/kg to about 1×10¹¹ cells/kg 10⁴ and at or about 10¹¹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight. In other exemplary embodiments, a suitable dosage range of modified cells for use in a method of the present disclosure includes, without limitation, from about 1×10⁵ cells/kg to about 1×10⁶ cells/kg, from about 1×10⁶ cells/kg to about 1×10⁷ cells/kg, from about 1×10⁷ cells/kg about 1×10⁸ cells/kg, from about 1×10⁸ cells/kg about 1×10⁹ cells/kg, from about 1×10⁹ cells/kg about 1×10¹⁰ cells/kg, from about 1×10¹⁰ cells/kg about 1×10¹¹ cells/kg. In an exemplary embodiment, a suitable dosage for use in a method of the present disclosure is about 1×10⁸ cells/kg. In an exemplary embodiment, a suitable dosage for use in a method of the present disclosure is about 1×10⁷ cells/kg. In other embodiments, a suitable dosage is from about 1×10⁷ total cells to about 5×10⁷ total cells. In some embodiments, a suitable dosage is from about 1×10⁸ total cells to about 5×10⁸ total cells. In some embodiments, a suitable dosage is from about 1.4×10⁷ total cells to about 1.1×10⁹ total cells. In an exemplary embodiment, a suitable dosage for use in a method of the present disclosure is about 7×10⁹ total cells.

In some embodiments, the cells are administered at or within a certain range of error of between at or about 10⁴ and at or about 10⁹ CD4⁺ and/or CD8⁺ cells/kilograms (kg) body weight, such as between 10⁵ and 10⁶ CD4⁺ and/or CD8⁺ cells/kg body weight, for example, at or about 1×10⁵ CD4⁺ and/or CD8⁺ cells/kg, 1.5×10⁵ CD4⁺ and/or CD8⁺ cells/kg, 2×10⁵ CD4⁺ and/or CD8⁺ cells/kg, or 1×10⁶ CD4⁺ and/or CD8⁺ cells/kg body weight. In some embodiments, the cells are administered at or within a certain range of error of, greater than, and/or at least about 1×10⁶, about 2.5×10⁶, about 5×10⁶, about 7.5×10⁶, or about 9×10⁶ CD4⁺ cells, and/or at least about 1×10⁶, about 2.5×10⁶, about 5×10⁶, about 7.5×10⁶, or about 9×10⁶ CD8⁺ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4⁺ cells, and/or between about 10⁸ and 10¹² or between about 10¹⁰ and 10¹¹ CD8⁺ cells.

In some embodiments, the cells are administered at or within a tolerated range of a desired output ratio of multiple cell populations or sub-types, such as CD4⁺ and CD8⁺ cells or sub-types. In some aspects, the desired ratio can be a specific ratio or can be a range of ratios, for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, a dose of modified cells is administered to a subject in need thereof, in a single dose or multiple doses. In some embodiments, a dose of modified cells is administered in multiple doses, e.g., once a week or every 7 days, once every 2 weeks or every 14 days, once every 3 weeks or every 21 days, once every 4 weeks or every 28 days. In an exemplary embodiment, a single dose of modified cells is administered to a subject in need thereof. In an exemplary embodiment, a single dose of modified cells is administered to a subject in need thereof by rapid intravenous infusion.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

In certain embodiments, the modified cells of the invention may be administered to a subject in combination with an immune checkpoint antibody (e.g., an anti-PD1, anti-CTLA-4, or anti-PDL1 antibody). For example, the modified cell may be administered in combination with an antibody or antibody fragment targeting, for example, PD-1 (programmed death 1 protein). Examples of anti-PD-1 antibodies include, but are not limited to, pembrolizumab (KEYTRUDA®, formerly lambrolizumab, also known as MK-3475), and nivolumab (BMS-936558, MDX-1106, ONO-4538, OPDIVA®) or an antigen-binding fragment thereof. In certain embodiments, the modified cell may be administered in combination with an anti-PD-L1 antibody or antigen-binding fragment thereof. Examples of anti-PD-L1 antibodies include, but are not limited to, BMS-936559, MPDL3280A (TECENTRIQ®, Atezolizumab), and MED14736 (Durvalumab, Imfinzi). In certain embodiments, the modified cell may be administered in combination with an anti-CTLA-4 antibody or antigen-binding fragment thereof. An example of an anti-CTLA-4 antibody includes, but is not limited to, Ipilimumab (trade name Yervoy). Other types of immune checkpoint modulators may also be used including, but not limited to, small molecules, siRNA, miRNA, and CRISPR systems. Immune checkpoint modulators may be administered before, after, or concurrently with the modified cell comprising the CAR. In certain embodiments, combination treatment comprising an immune checkpoint modulator may increase the therapeutic efficacy of a therapy comprising a modified cell of the present invention.

In some embodiments, the compositions of the invention comprising the spacer molecule are administered at a desired dosage, which in some aspects includes a desired dose of PEG. Thus, the dosage of cells in some embodiments is based on a total amount of spacer molecule (or weight per kg body weight). In some embodiments, the dosage of the composition is based on a desired total number (or amount per kg of body weight) of spacer molecule in the individual populations or of individual cell types. In some embodiments, the administration of the spacer molecule is at a dose of between about 10 mg/kg and about 100 mg/kg (e.g., 10 mg/kg, 50 mg/kg, or 100 mg/kg). In an exemplary embodiment, the dose of spacer molecule is about 100 mg/kg.

Following administration of the cells and/or the composition comprising the spacer molecule, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In certain embodiments, the subject is provided a secondary treatment. Secondary treatments include but are not limited to chemotherapy, radiation, surgery, and medications.

In some embodiments, the subject can be administered a conditioning therapy prior to CAR T cell therapy. In some embodiments, the conditioning therapy comprises administering an effective amount of cyclophosphamide to the subject. In some embodiments, the conditioning therapy comprises administering an effective amount of fludarabine to the subject. In preferred embodiments, the conditioning therapy comprises administering an effective amount of a combination of cyclophosphamide and fludarabine to the subject.

Administration of a conditioning therapy prior to CAR T cell therapy may increase the efficacy of the CAR T cell therapy. Methods of conditioning patients for T cell therapy are described in U.S. Pat. No. 9,855,298, which is incorporated herein by reference in its entirety.

In some embodiments, a specific dosage regimen of the present disclosure includes a lymphodepletion step prior to the administration of the modified T cells. In an exemplary embodiment, the lymphodepletion step includes administration of cyclophosphamide and/or fludarabine.

In some embodiments, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day). In an exemplary embodiment, the dose of cyclophosphamide is about 300 mg/m²/day. In some embodiments, the lymphodepletion step includes administration of fludarabine at a dose of between about 20 mg/m²/day and about 900 mg/m²/day (e.g., 20 mg/m²/day, 25 mg/m²/day, 30 mg/m²/day, or 60 mg/m/day). In an exemplary embodiment, the dose of fludarabine is about 30 mg/m²/day.

In some embodiment, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the lymphodepletion step includes administration of cyclophosphamide at a dose of about 300 mg/m²/day, and fludarabine at a dose of about 30 mg/m²/day.

In an exemplary embodiment, the dosing of cyclophosphamide is 300 mg/m²/day over three days, and the dosing of fludarabine is 30 mg/m²/day over three days.

Dosing of lymphodepletion chemotherapy may be scheduled on Days −6 to −4 (with a −1-day window, i.e., dosing on Days −7 to −5) relative to T cell (e.g., CAR-T, TCR-T, a modified T cell, etc.) infusion on Day 0.

In an exemplary embodiment, for a subject having cancer, the subject receives lymphodepleting chemotherapy including 300 mg/m² of cyclophosphamide by intravenous infusion 3 days prior to administration of the modified T cells. In an exemplary embodiment, for a subject having cancer, the subject receives lymphodepleting chemotherapy including 300 mg/m² of cyclophosphamide by intravenous infusion for 3 days prior to administration of the modified T cells.

In an exemplary embodiment, for a subject having cancer, the subject receives lymphodepleting chemotherapy including fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including fludarabine at a dose of 30 mg/m² for 3 days.

In an exemplary embodiment, for a subject having cancer, the subject receives lymphodepleting chemotherapy including cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including cyclophosphamide at a dose of about 300 mg/m²/day, and fludarabine at a dose of 30 mg/m² for 3 days.

Cells of the invention can be administered in dosages and routes and at times to be determined in appropriate pre-clinical and clinical experimentation and trials. Cell compositions may be administered multiple times at dosages within these ranges.

Administration of the cells of the invention may be combined with other methods useful to treat, ameliorate, and/or prevent the desired disease or condition as determined by those of skill in the art.

It is known in the art that one of the adverse effects following infusion of CAR T cells is the onset of immune activation, known as cytokine release syndrome (CRS). CRS is immune activation resulting in elevated inflammatory cytokines. CRS is a known on-target toxicity, development of which likely correlates with efficacy. Clinical and laboratory measures range from mild CRS (constitutional symptoms and/or grade-2 organ toxicity) to severe CRS (sCRS; grade ≥3 organ toxicity, aggressive clinical intervention, and/or potentially life threatening). Clinical features include: high fever, rigors, malaise, fatigue, myalgia, nausea, anorexia, tachycardia/hypotension, hypotension, headache, aphasia, disorientation, lethargy, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, IL-5, IL-13, and IL-6 have been shown following CAR T-cell infusion. By way of a non-limiting example, one CRS signature is elevation of cytokines including IL-6 (severe elevation), IFN-gamma, TNF-alpha (moderate), and IL-2 (mild). Elevations in clinically available markers of inflammation including ferritin and C-reactive protein (CRP) have also been observed to correlate with the CRS syndrome. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, the invention includes, following the diagnosis of a car-related toxicity such as CRS. the administration of a composition comprising a spacer molecule to the subject, such that the spacer molecule becomes conjugated to the modified immune cells, which reduce their interaction with endogenous immune cells, thereby eliminating or reducing the severity of the toxicity. It is also contemplated that the administration of the spacer molecule can be combined with other common clinical strategies to treat, ameliorate, minimize, and/or avoid CAR related toxicities known in the art. For example, systemic corticosteroids may be administered to rapidly reverse symptoms of sCRS (e.g., grade 3 CRS) without compromising initial antitumor response.

In some embodiments, an anti-IL-6R antibody may also be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

Features consistent with Macrophage Activation Syndrome (MAS) or Hemophagocytic lymphohistiocytosis (HLH) have been observed in patients treated with CAR-T therapy (Henter, 2007), coincident with clinical manifestations of the CRS. MAS appears to be a reaction to immune activation that occurs from the CRS, and should therefore be considered a manifestation of CRS. MAS is similar to HLH (also a reaction to immune stimulation). The clinical syndrome of MAS is characterized by high grade non-remitting fever, cytopenias affecting at least two of three lineages, and hepatosplenomegaly. It is associated with high serum ferritin, soluble interleukin-2 receptor, and triglycerides, and a decrease of circulating natural killer (NK) activity.

### Pharmaceutical Compositions and Formulations

Also provided are populations of modified immune cells of the invention, compositions containing such cells and/or enriched for such cells, such as in which cells expressing the recombinant receptor make up at least 50%, 60%, 70%, 80%, 90%, 91%, 92%, 93%, 94%, 95%, 96%, 97%, 98%, 99%, or more of the total cells in the composition or cells of a certain type such as T cells or CD8⁺ or CD4⁺ cells. Among the compositions are pharmaceutical compositions and formulations for administration, such as for adoptive cell therapy. Also provided are therapeutic methods for administering the cells and compositions to subjects, e.g., patients.

Also provided are compositions including the modified immune cells for administration, including pharmaceutical compositions and formulations, such as unit dose form compositions including the number of cells for administration in a given dose or fraction thereof. Also provided are compositions including a spacer molecule (e.g., polyethylene glycol (PEG) or derivative thereof) for administration, including pharmaceutical compositions and formulations, such as unit dose form compositions including the amount of spacer molecule (e.g., PEG or derivative thereof) for administration in a given dose or fraction thereof.

The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made, and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2nd edition).

The material and methods employed in the experimental examples disclosed herein are now described.

Chemicals. polyethylene glycol (PEG) with molecular weight of 1 k, 5 k, 10 k, 100 k, and 600 k were purchased from Sigma. DBCO-acid was purchased from Sigma. N-azidoacetylmannosamine-tetraacylated (Catalog #88904) was obtained from Fisher. CD19 CAR plasmid is a gift from Scott McComb (Addgene #135991). The packaging plasmids pMD2.G (Addgene #12259) and psPAX2 (Addgene #12260) were from Didier Trono. CSFE dye and e450 dye were purchased from Fisher. FirePlex®-96 Key Cytokines (Human) Immunoassay Panel (Catalog #ab243549) was purchased from Abcam. ELISA kit for mouse serum amyloid A (Catalog #KMA0021) was obtained from Thermo Fisher. Human CD68 antibody (Catalog #764375) was purchased from Cell Signaling Technology. Anti-CD3 antibody (Catalog #85061) for T cell binding experiment was obtained from CST. StemCell EasySep human T cell isolation kit (Catalog #17951), Dynabeads™ Human T-Activator CD3/CD28 (Catalog #11132D), retronectin (TAKARA Bio, Catalog #T100B), Lipofectamine 2000 (Catalog #11668019) were obtained from Thermo Fisher.

Cell lines and animals. Raji-Luc-GFP cell line is a gift from Prof. Alan Epstein. 293T cells were purchased from ATCC. The cells were tested mycoplasma negative before use. Human fetal liver CD34⁺ cells were purchased from the Tissue Bank of University of Pennsylvania. Triple transgenic NSG (SGM3) mice expressing human stem cell factor, granulocyte-macrophage colony-stimulating factor (GM-CSF) and IL-3 were obtained from the Jackson lab and were housed in a specific pathogen free-grade animal facility with air humidity 40%-70%, ambient temperature (22±2 °C), and 12-h dark/12-h light cycle. All protocols performed on animals in this study were approved by the institutional animal care and use committee of the University of Pennsylvania.

Lentivirus production. 293T cells were cultured in 10 cm dish and after 80% confluence was reached, the cells were treated with Opti-MEM medium containing 80 μL lipofectamine 2000 mixed with CAR plasmid (10 μg), and two packaging plasmids (pCMV-VSV-G, 7.5 μg; psPAX2, 5 g) loading). After 6 h, old medium was removed and 8 ml of pre-warmed medium was added very gently without disturbing the cells. After 24 h, virus-containing supernatant was collected and was passed through a 0.45 um filter. Virus-containing supernatant was then aliquoted and stored at −80 °C for further use.

Preparation of CAR T cells. 24-well untreated plates were treated with 20 μg/mL of Retronectin in PBS at 4 °C overnight. Then lentiviral supernatants were added, and the plate was centrifuged at 1000 g for 1 h for lentiviral adhesion. The spleen of humanized mice was collected and CD8⁺ T cells were isolated using a human CD8⁺ T cell isolation kit. T cells were activated with Dynabeads™ Human T-Activator CD3/CD28 at a bead-to-cell ratio of 1:1, and 50 IU/mL recombinant IL-2 was added to the cell culture medium. After 48 h of activation, Dynabeads were removed and the T cells were added to lentiviral-coated 24-well plates (1 million cells per well). 2 days after T cells were added, transduction efficiencies were determined by flow.

Synthesis of DBCO-PEG. Dibenzocyclooctyne (DBCO)-acid was mixed with PEG 1 k, 5 k, 10 k, 100 k, or 600 k in DCM under N₂ protection, EDC and DMAP were used as catalysts. After 24 h, DCM was removed under reduced pressure and the free DBCO-acid and catalysts were removed by dialysis.

Azido glycans modification on CAR T cell surfaces. CAR T cell surface was labeled with azido glycans by culturing CAR T cells with Ac4ManNAz for 48 h.

Construction of humanized immune system NSG-SGM3 mouse model. Previous studies using murine models of CRS have found that CAR T cell injected to humanized mice bearing leukemia can induce severe CRS and neurotoxicity. Here, a humanized mice model was also constructed following these previously published method with several modifications. NSG-SGM3 mice were treated with busulfan (40 mg/kg) to deplete bone marrow cells followed by i.v. injection with 10⁵ human fetal liver CD34⁺ cells. Xenogeneic graft-versus-host disease (X-GVHD) was monitored for daily by assessing individual animal activity, weight loss, fur texture, and skin integrity. CRS mortality was defined as death preceded by the following criteria: >20% body weight loss, ΔT >2 °C and serum IL-6 >2,500 pg/mL. Neurotoxicity-induced lethality was defined as death in the absence of CRS criteria and preceded by either seizures or paralysis.

Flow and cell sorting experiments. 50-100 μL mouse blood was collected in EDTA-pretreated tubes using the orbital bleeding method. Cells were spun down, the red blood cells were lysed with ACK lysing buffer, and the cell suspensions were passed through a 70 μm cell strainer before being stained with antibodies for flow (BD LSR). CAR T cell sorting from mouse blood was performed in BD AriaFusion ES.

IVIS® imaging. Raji-Luc-GFP tumor growth in mice was monitored use a Perkin Elmer in vivo imaging system (IVIS®, Lumina 3). Mice were i.p. injected with d-luciferin (150 mg/kg) for 15 min and then were imaged in the IVIS® system under inhaled isoflurane anesthesia.

Statistics. Statistical analysis was performed in Graphpad prism 7.0 software. Error bars represent means±standard deviation (s.d.). A two-tailed Student's t-test was used to calculate the statistical differences between two groups. The differences in animal survival experiments were calculated using the Kaplan-Meier method, and the P values were determined using the log-rank test.

The experimental examples are now described.

### Example 1: PEGylation of CAR T Cells can Block Cell-to-Cell Interactions and Reduce Cytokine Release by Monocytes In Vitro

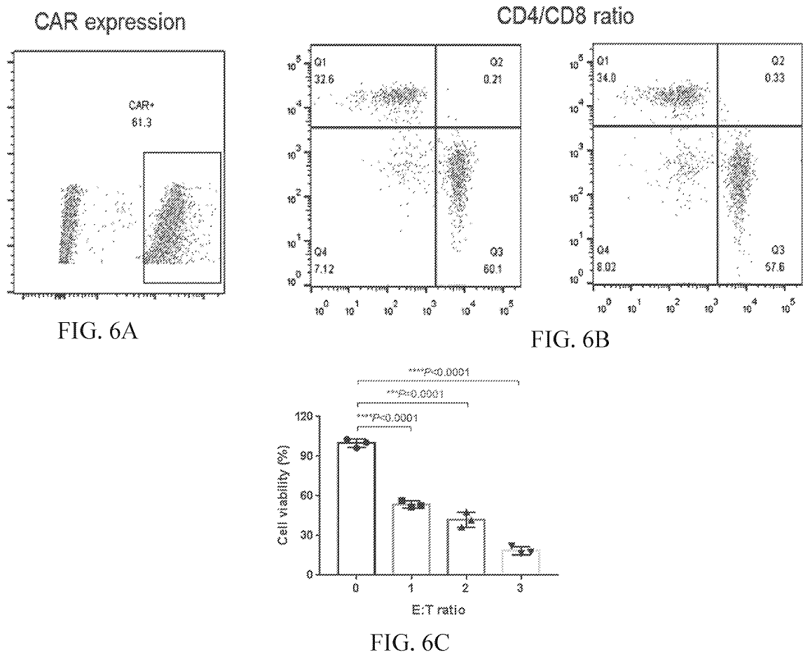

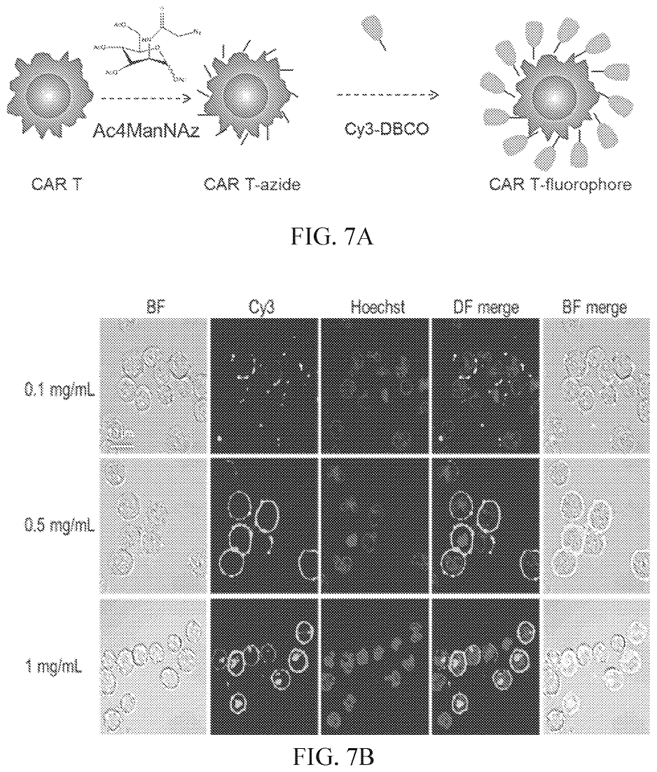

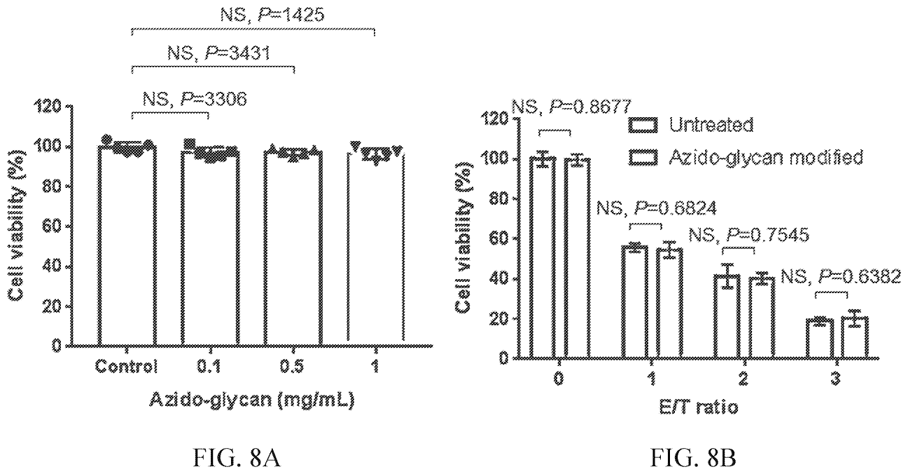

Polyethylene glycol (PEG) was chosen as a biologically inert, nontoxic, polymeric spacer because it is an FDA approved biomaterial and is widely used in drug delivery applications. In order to generate PEG-coated CAR T cells, human CD19 targeted CAR T cells (1928z CAR) were first prepared using a previously reported method. CAR expression was confirmed by flow cytometry and the ratio of CD8/CD4 T cells did not change following CAR transduction (FIG. 6). Metabolic labeling was used to label the surface of CAR T cells with azide groups by culturing CAR T cells with azido glycans (N-azidoacetylmannosamine-tetraacylated, Ac4ManNAz) for 48 h (FIG. 7). Azido glycan uptake and incorporation into cell-surface sialylated glycans on CAR T cells was found to be non-toxic (FIG. 8A), and did not affect the tumor cell killing capacity of CAR T cells (FIG. 8B).

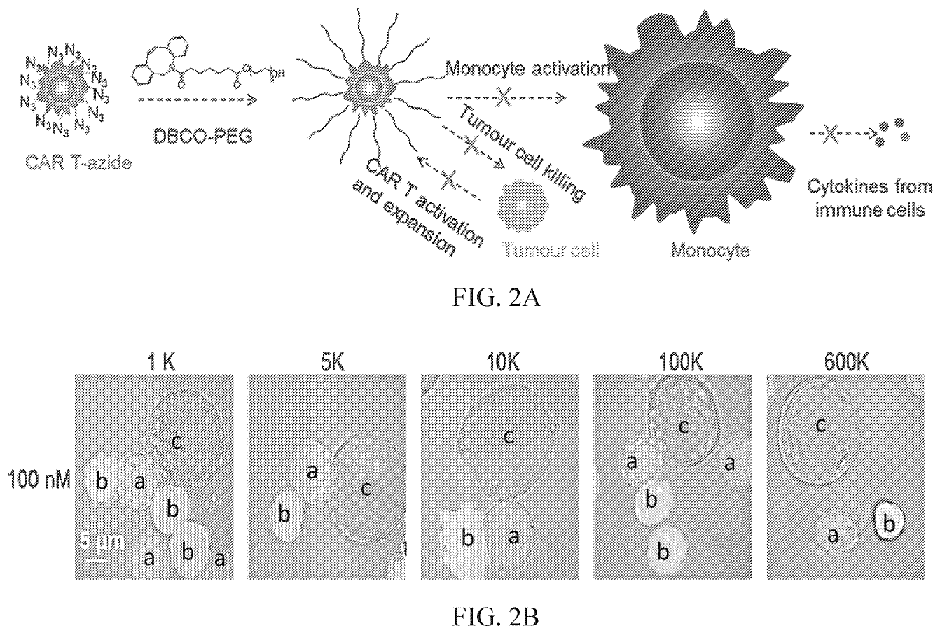

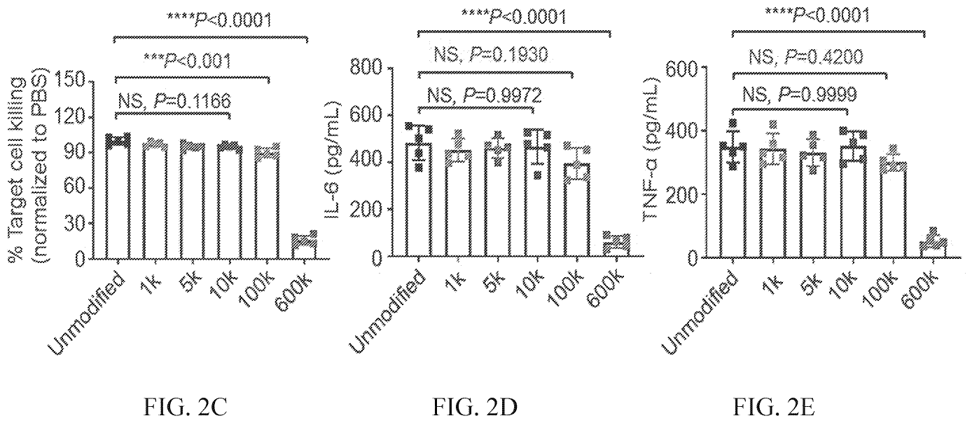

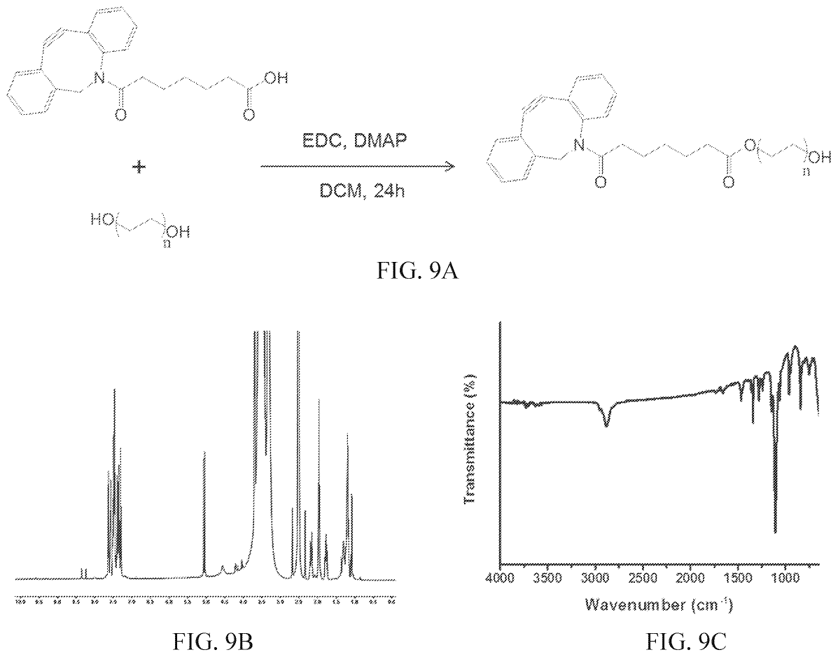

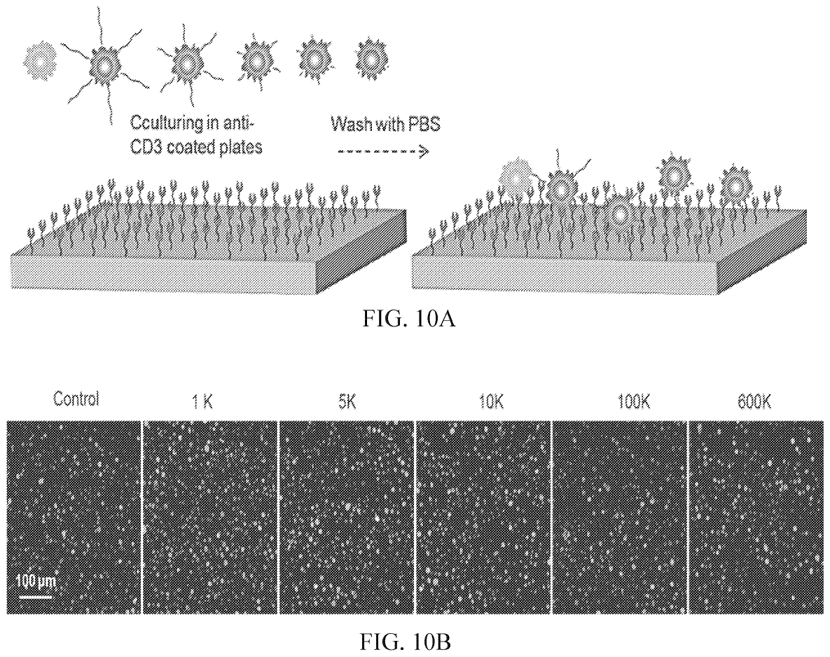

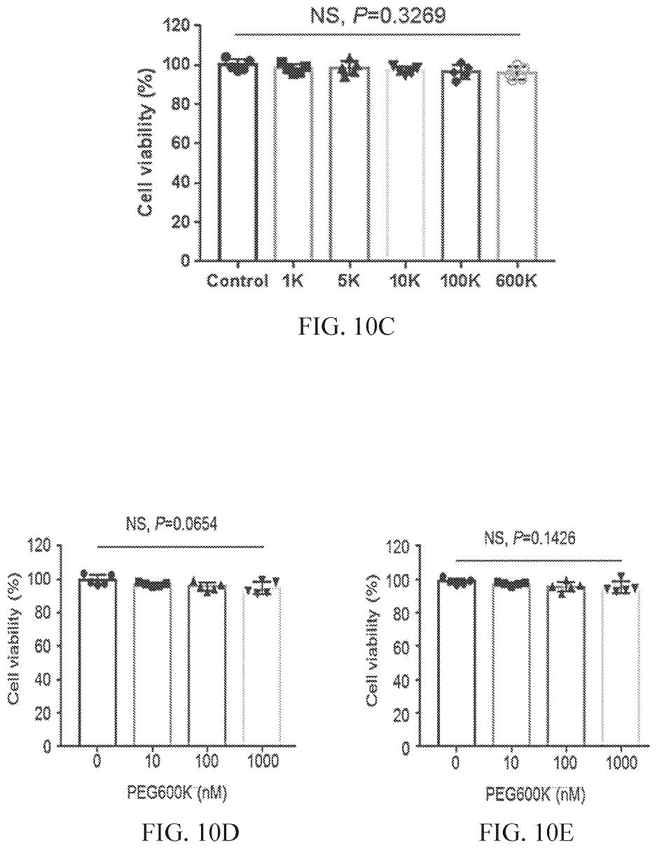

To conjugate PEG to azide-labeled CAR T cells, dibenzocyclooctyne (DBCO)-acid was attached to PEG to form a DBCO-PEG conjugate (FIG. 9). The DBCO group reacts with the azide group present on the CAR T cell surface under culture conditions (FIG. 2A) or in living animals. 100 nM DBCO-PEG of molecular weights (MW) ranging from 1 k, 5 k, 10 k, 100 k, and 600 k was used to modify CAR T cells (FIG. 2B). An in vitro binding experiment using anti-CD3 antibody-coated plates was used to assess how interactions with PEGylated CAR T cells were affected. PEGylated CAR T cells with PEGs of MW 1 k, 5 k, 10 k, and 100 k were able to bind to anti-CD3 antibody-coated 96-well plates, but PEGylated CAR T cells with a PEG of MW 600 k failed to bind to the well plate. This result demonstrated that PEG 600k-modified CAR T cells can potentially be used to block cell-to-cell interactions (FIG. 10A, 10B). To assess how PEGylation may prevent CAR T cells from interacting with target cells and monocytes, PEGylated CAR T cells, Raji tumor cells, and monocytes were incubated together and examined using confocal microscopy. Confocal images showed that PEGylated CAR T cells with PEGs of MW 1 k, 5 k, 10 k, and 100 k failed to block interactions between CAR T cells, Raji tumor cells and monocytes, whereas PEGylated CAR T cells with a PEG of MW 600 k substantially blocked all cell-to-cell interactions (FIG. 2B). PEG600k has a length of about 6 μm, which is much longer than the other PEGs tested and may contribute to increased steric hindrance in blocking cell-to-cell interactions.

These studies revealed that CAR T cell modification with PEG600k also greatly decreased target tumor cell killing (FIG. 2C). Interestingly the levels of IL-6, a cytokine secreted by activated monocytes, and TNF-α, a cytokine secreted by activated CAR T cells, were also substantially decreased (FIGS. 2D, 2E). Further, PEG600k modification did not induce toxicity to the cells (FIGS. 10D, 10E).These results demonstrate that modification of CAR T cells with PEG600k can block cell-to-cell interactions and decrease both monocyte activation and cytokine release in vitro. Encouraged by this data, studies then sought to test if PEGylation of CAR T cells in vivo may reduce CRS.

### Example 2: In Situ PEGylation of CAR T Cells Alleviates Cytokine Release Syndrome in an In Vivo CRS Model

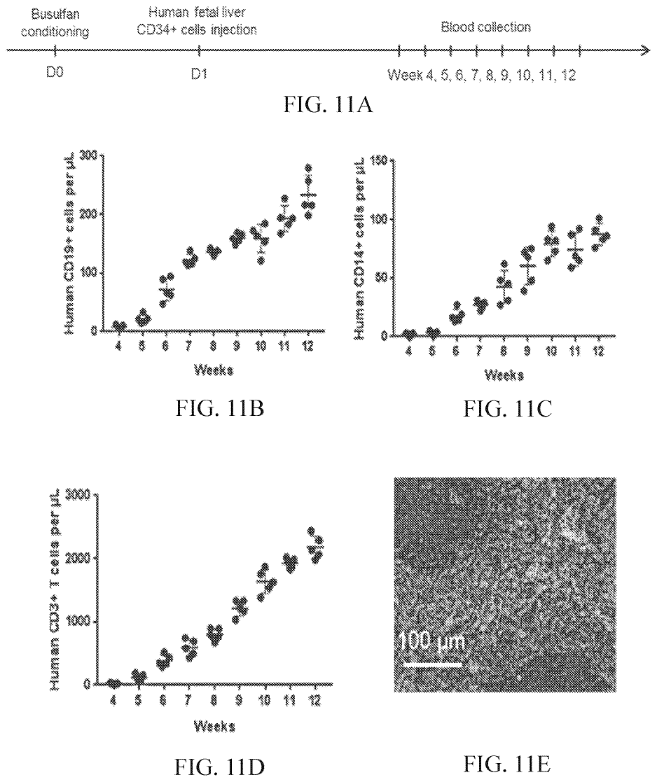

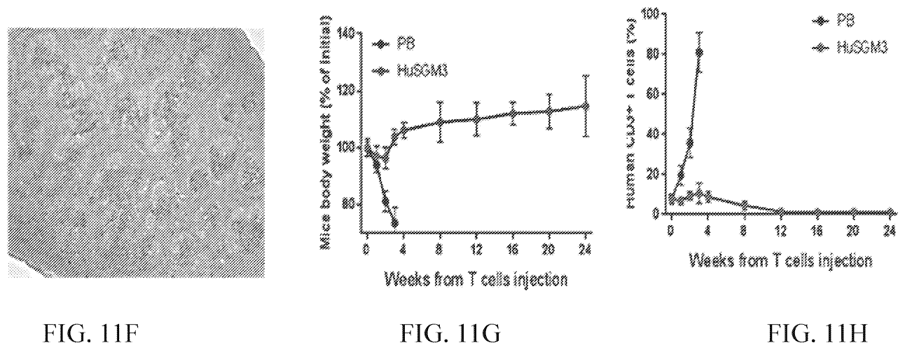

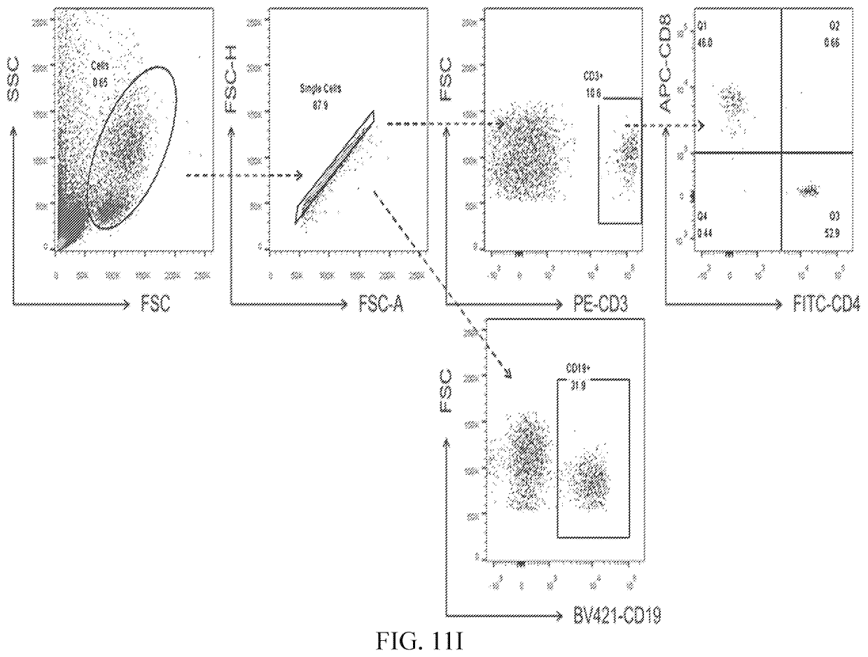

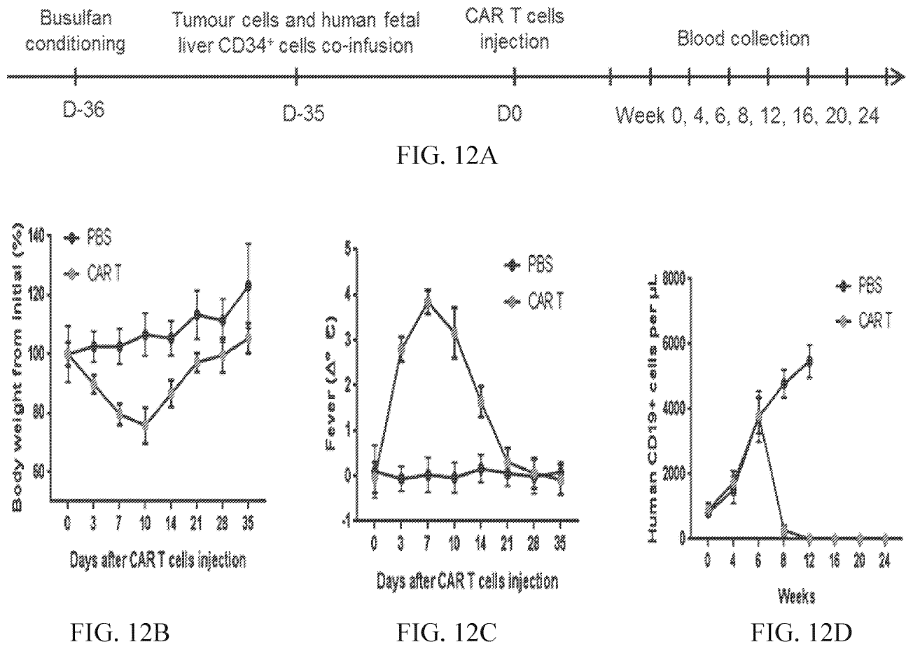

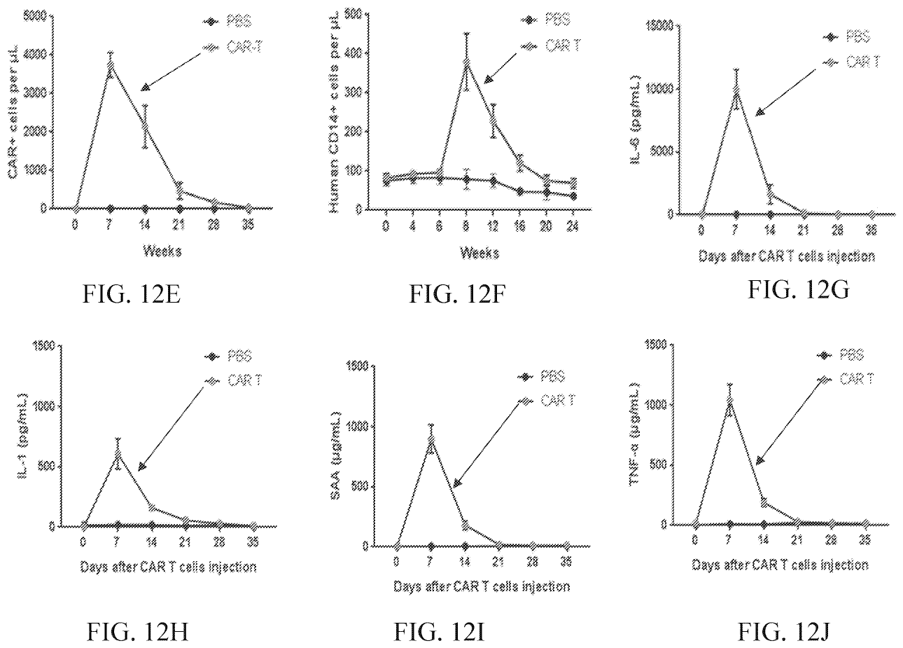

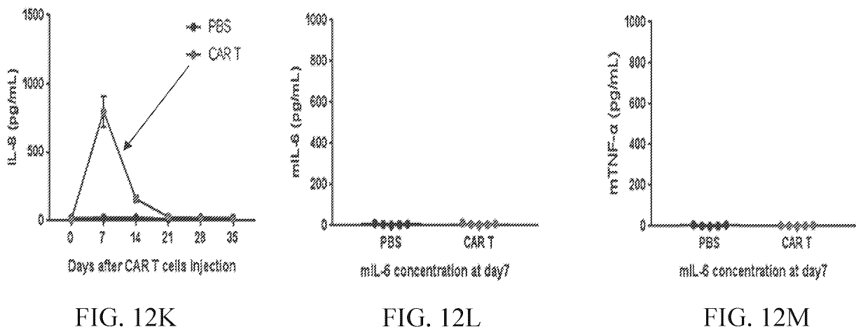

A humanized mouse model was constructed following an approach from a previous report with a slight modification (see Methods section, FIG. 11A). The development of human immune cells including T cells, B cells, and monocytes in blood was determined by flow cytometry and histological analysis (FIGS. 11B-11F). Adoptive transfer of T cells from one humanized mouse to an immunocompromised NSG-SGM3 mouse did not induce xenogeneic graft-versus-host disease (X-GVHD, FIGS. 11G-11H). This suggests that CAR T cells prepared using T cells from humanized mice are suitable for adoptive transfer applications. To construct the CRS model in tumor bearing mice, 1×10⁴ Raji cells and 1×10⁵ human fetal liver CD34⁺ cells were co-infused into NSG-SGM3 mice (FIG. 12A) to develop human immune cells and to allow tumor growth. After 7 weeks, 2×10⁶ human T cells isolated from a non-tumor-bearing humanized mouse were transduced with CD19 CAR (1928z). These cells were then injected intravenously into tumor-bearing mice. Body weight and temperature (FIGS. 12B, 12C) were recorded every three days. Blood was also collected to determine the levels of various cytokines and the numbers of tumor cells, CAR T cells, and monocytes (FIGS. 12D-12F). Although injection of CAR T cells greatly decreased CD19⁺ cell numbers in blood (FIG. 12D), there were several CRS-related symptoms, such as decreased body weight and high fever (FIGS. 12B, 12C). Subsequent observations found that CAR T cells expanded rapidly after injection and also observed a remarkable increase in the number of CD14⁺ monocytes (FIGS. 12E, 12F). The concentrations of several human cytokines, such as IL-6, IL-1, TNF-α, IL-8, and mouse serum amyloid A (SAA, a murine homolog to the human CRS biomarker C-reactive protein) in the blood were also monitored (FIGS. 12G-12K). Injection of CAR T cells induced rapid generation of human T cell-derived cytokine TNF-α and human monocyte-derived cytokines, such as IL-6, IL-1, IL-8, as well as mouse SAA (FIGS. 12J-12K). Mouse IL-6 and TNF-α levels were undetectable (FIGS. 12L, 12M), which is consistent with previous reports. Collectively, these results demonstrate successful construction of the CRS model in humanized mice.

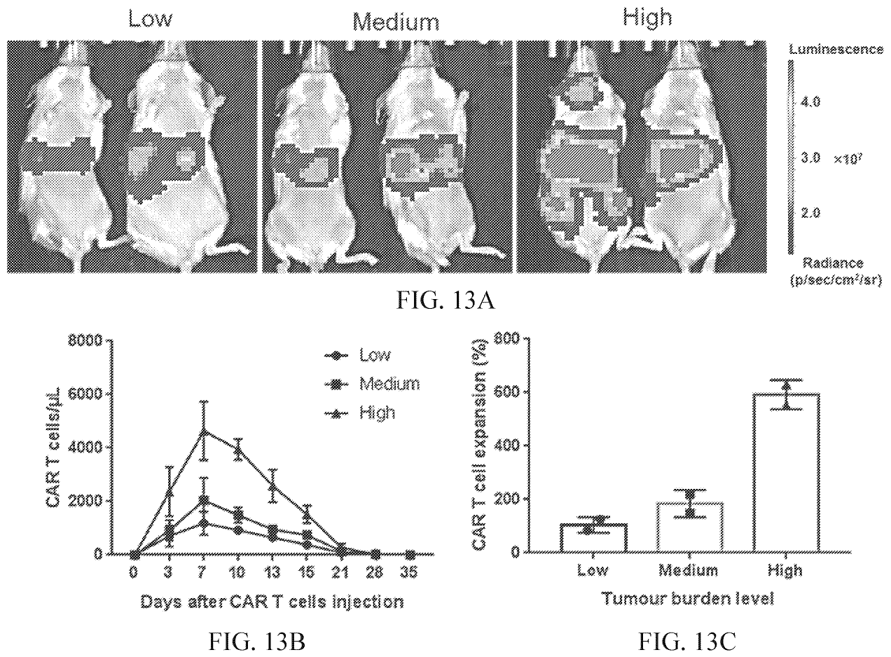

Before testing whether DBCO-PEG600k can alleviate CRS in vivo, the expansion levels of CAR T cells in vivo was first evaluated. Because azide-labeled CAR T cells may divide after infusion, the azide groups on the cell surface can be diluted and thus there may not be sufficient azides groups on the surface of CAR T cells for DBCO-PEG600k conjugation to induce steric hindrance effects. Previous studies have shown that CAR T cell expansion is dependent on tumor burden, so the expansion levels of CAR T cells was measured in response to three different tumor burden levels (FIG. 13). A higher tumor burden resulted in increased levels of CAR T cell expansion. In the low burden, medium burden and high burden groups, CAR T cells had expanded approximately 1.5, 2 and 6 times at day 7 (peak expansion), respectively (FIG. 13C). Second, studies evaluated how different rounds of expansion of azide-labeled CAR T cells influence PEGylation and the ability to bind to an anti-CD3 antibody-coated plate (FIG. 13D). After 1 or 6 rounds of expansion, PEGylation was unaffected and prevented binding of PEGylated CAR T cells to the antibody-coated plate (FIG. 13D). Moreover, 20 rounds of expansion achieved similar results (FIG. 13D). This can be attributed to the high concentration of azido-glycan used for the modification of CAR T cells in vitro. Together, these two findings demonstrate that CAR T cell expansion in vivo does not affect the formation of polymeric spacers using DBCO-PEG600k and that there are sufficient amounts of azide groups present for PEG conjugation after several rounds of CAR T cell expansion.

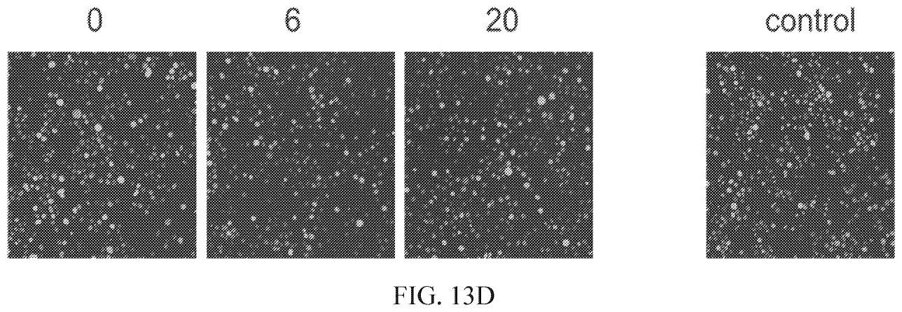

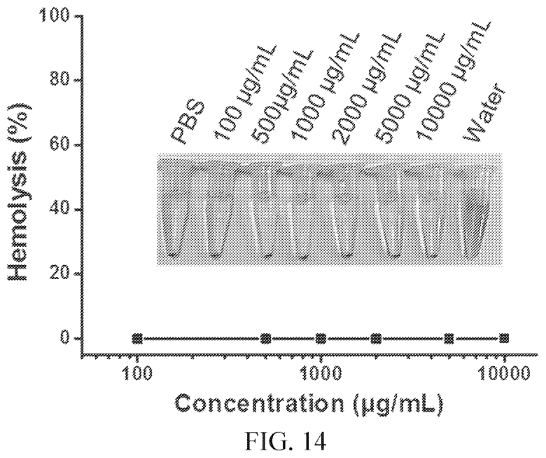

In order to evaluate the biocompatibility of DBCO-PEG600k, a hemolysis assay was conducted. Hemolysis was not induced at DBCO-PEG600k concentrations ranging from 100 μg/mL to 10 mg/mL, a range which covers the doses used in subsequent in vivo studies (FIG. 14).

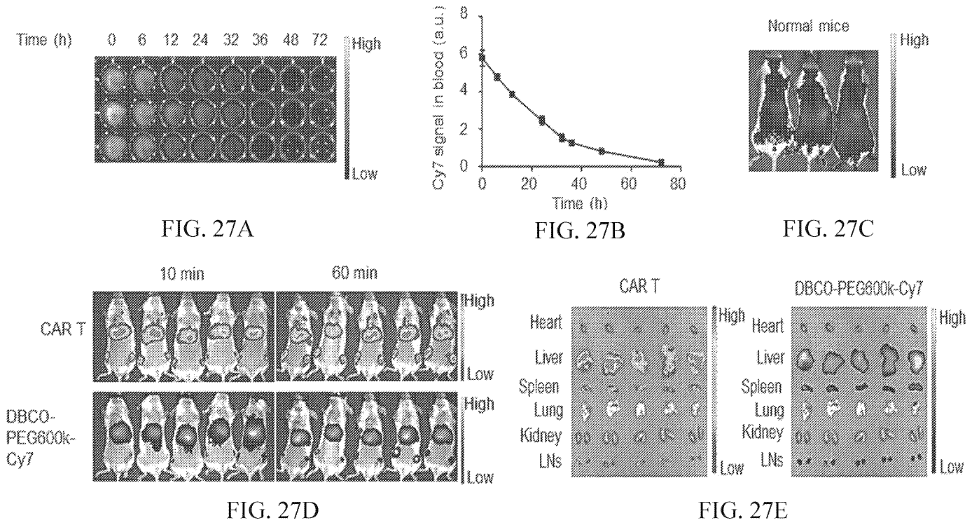

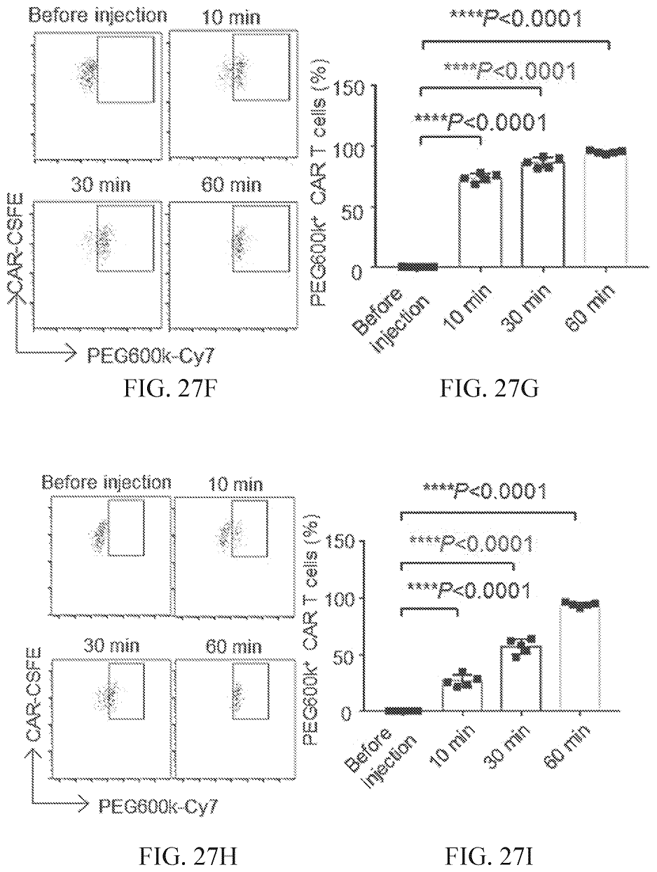

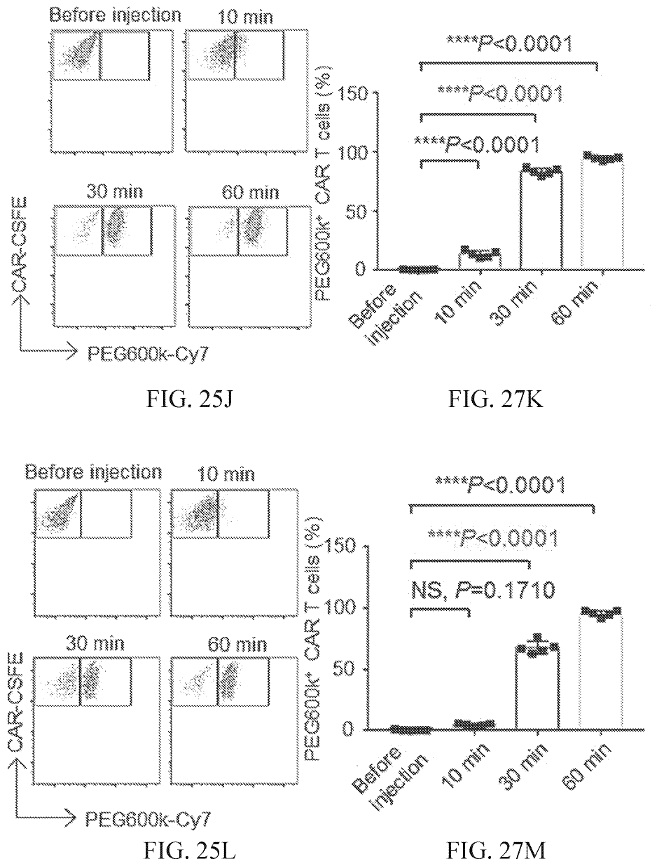

Studies then investigated the blood circulation of a Cy7-labeled DBCO-PEG600k (DBCO-PEG600k-Cy7) and found that DBCO-PEG600k-Cy7 has an 18 h half-life in the bloodstream in normal mice (FIGS. 27A, 27B), and it spreads to the whole body after injection (FIG. 2C), which is similar to previous reports. We also investigated if DBCO-PEG600k-Cy7 can conjugate to CSFE-modified CAR T-azide cells in vivo (FIGS. 27D-271). CAR T-CSFE cells were mainly detected in the liver, spleen and lymph nodes (FIGS. 27D, 27E), which are the tissues where tumor cells localize. Cy7 signal was also detected in these tissues, indicating potential conjugation of DBCO-PEG600k-Cy7 to CAR T-azide cells (FIGS. 27D, 27E). A flow cytometry experiment (FIGS. 27F-27M) showed that DBCO-PEG600k-Cy7 can conjugate to CAR T cells in the blood circulation, liver, spleen, and lymph node within 60 min (FIGS. 27F-27M). These results demonstrate the fast conjugation of DBCO-PEG600k to CAR T-azide cells in vivo.

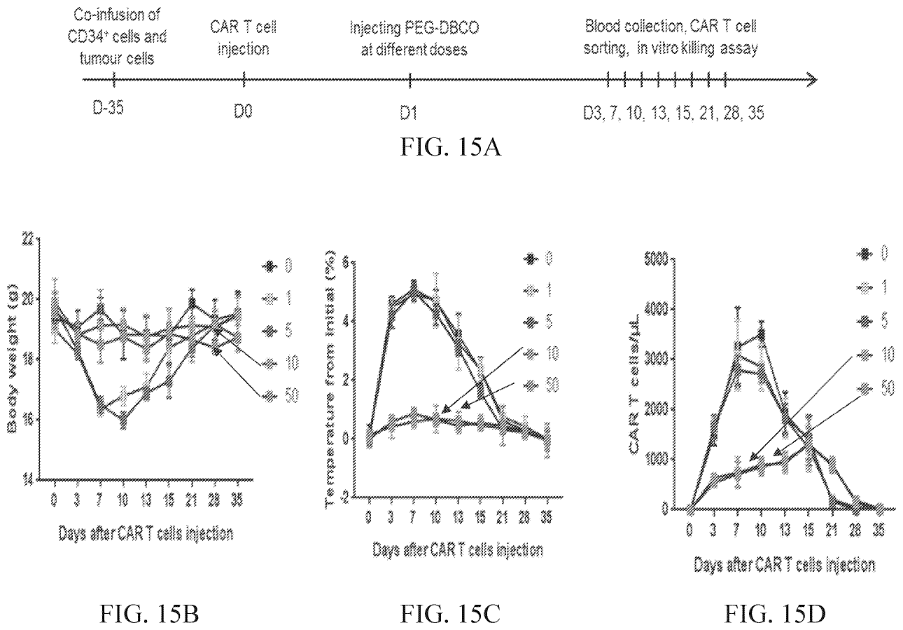

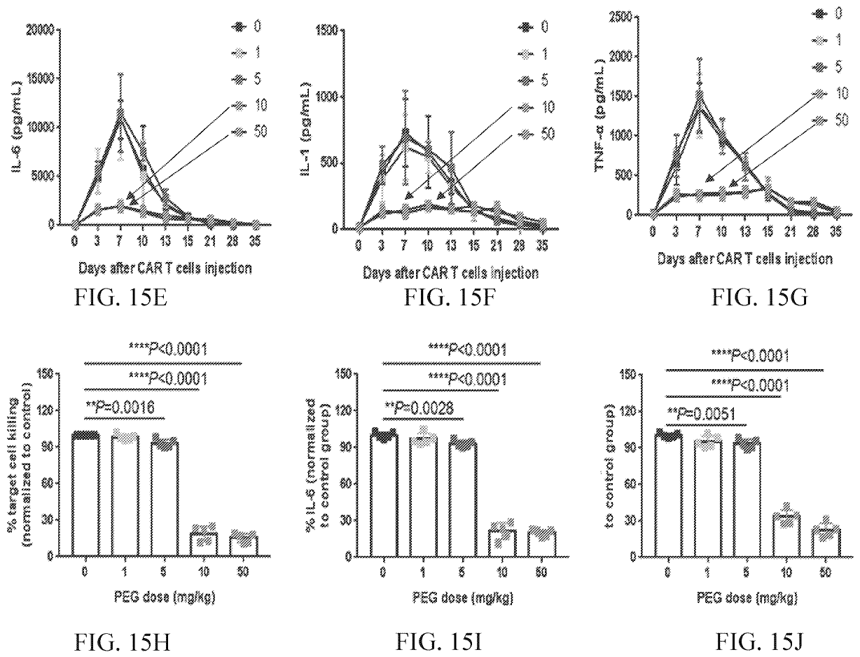

Next, a high tumor burden model was constructed by co-infusion of Raji tumor cells with CD34⁺ fetal liver cells at day −35. 2×10⁶ CAR T cells were intravenously infused at day 0 (FIG. 15A). After 24 h, all mice developed a high fever (ΔT>2 °C), which is one of the main symptoms of CRS. Immediately following high fever. DBCO-PEG600k at different doses (0, 1, 5, 10, 50 mg/kg) was intravenously administered to mice. Mouse body weight, temperature, and blood cytokines were documented over the course of 35 days (FIGS. 15B-15G). It was found that a low dose of DBCO-PEG600k (1 and 5 mg/kg) did not affect weight loss, body temperature and cytokine release compared to a PBS infusion (FIGS. 15B-15G). Excitingly, higher doses of DBCO-PEG600k (10 and 50 mg/kg) greatly reversed weight loss, high fever, and cytokine release (FIGS. 15B-15G). This is potentially because a higher density coating of DBCO-PEG600k on the surface of CAR T cells may block the interactions between CAR T cells, Raji tumor cells, and monocytes. To investigate this further, CAR T cells were sorted from mouse blood at day 7, and co-cultured with Raji-Luc-GFP cells and human monocytes for 24 h. CAR T cells from mice treated with 0, 1, and 5 mg/kg DBCO-PEG600k induced substantial Raji-Luc-GFP cell lysis and the production of both IL-6 and TNF-α. However, CAR T cells isolated from mice treated with 10 and 50 mg/kg DBCO-PEG600k exhibited very low levels of target cell killing and decreased cytokine production (FIG. 15H). Since injections of 10 mg/kg and 50 mg/kg of DBCO-PEG600k displayed similar effects in decreasing CRS symptoms, mice were dosed with 10 mg/kg of DBCO-PEG600k for all remaining studies. These results demonstrate that in situ PEGylation of CAR T cells can be utilized to potentially treat CRS.

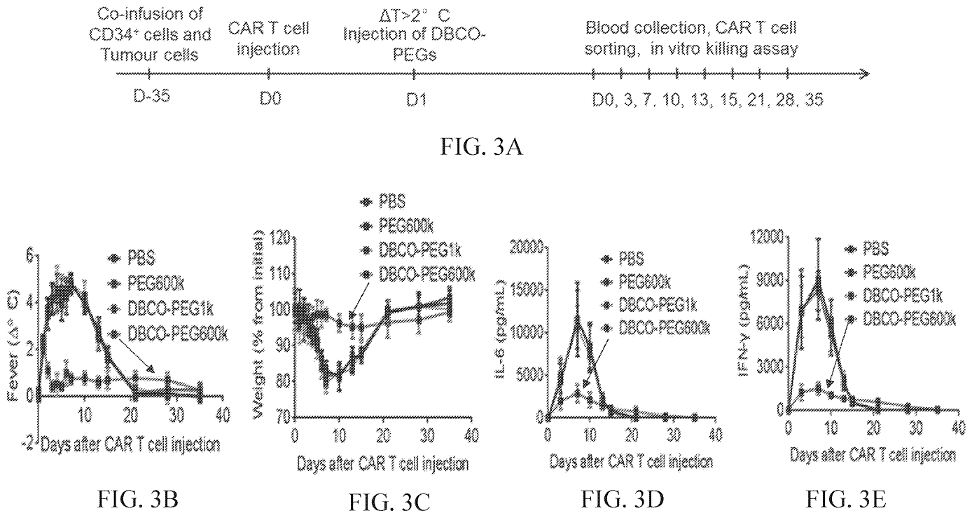

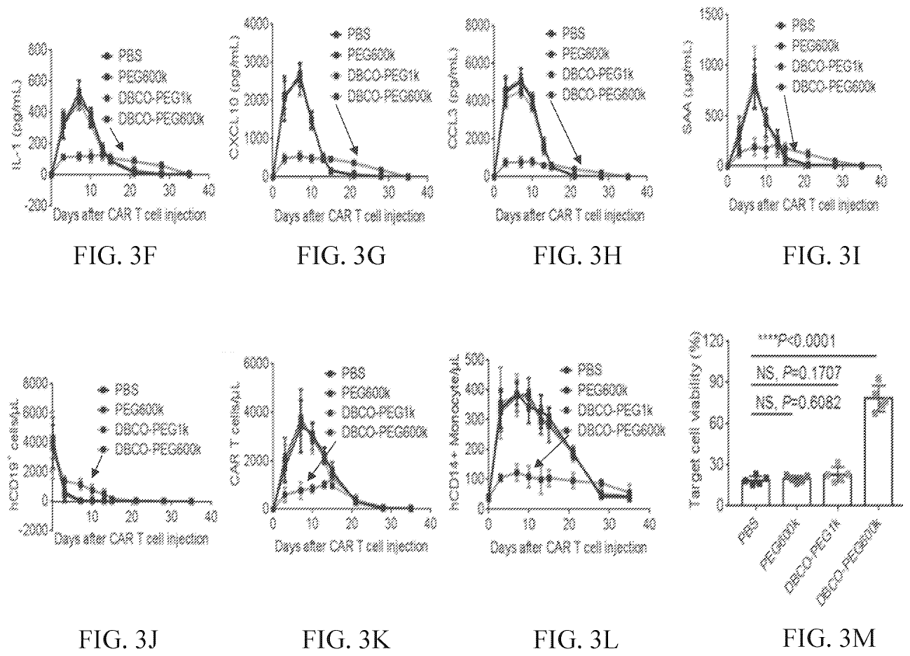

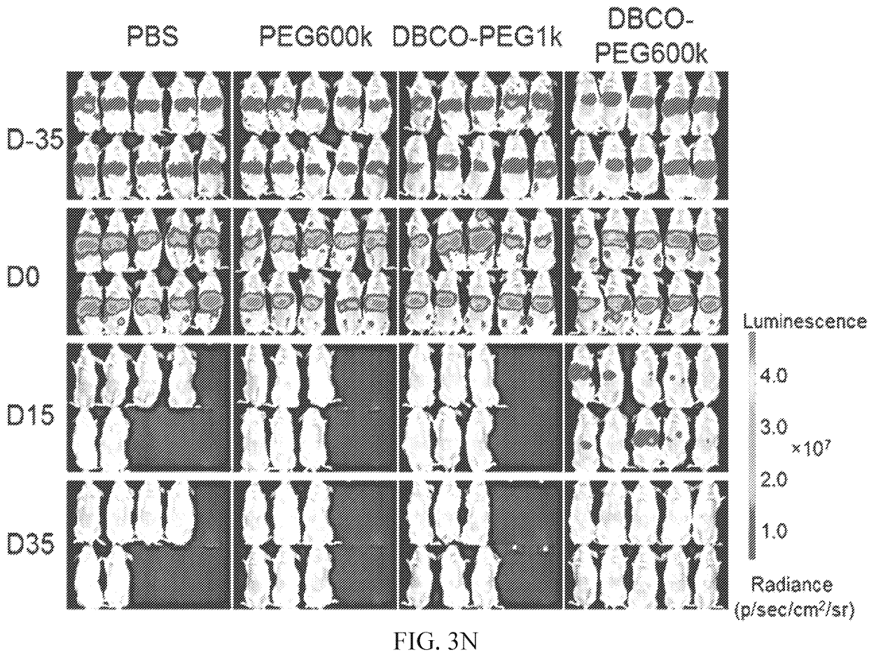

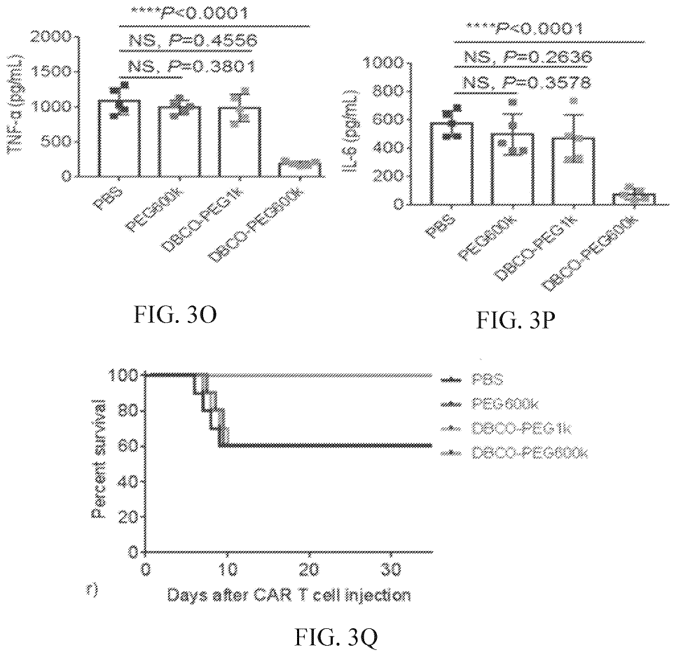

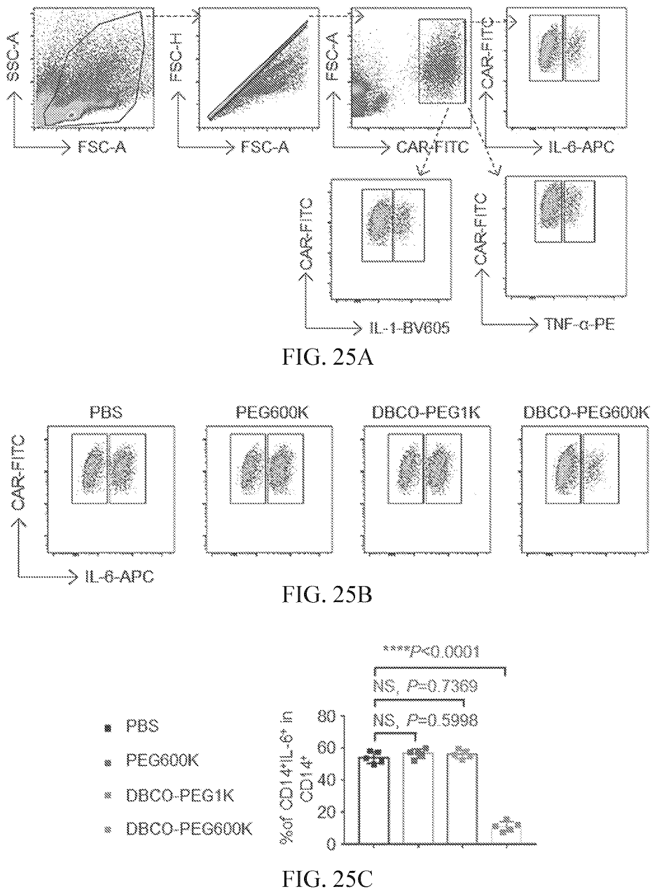

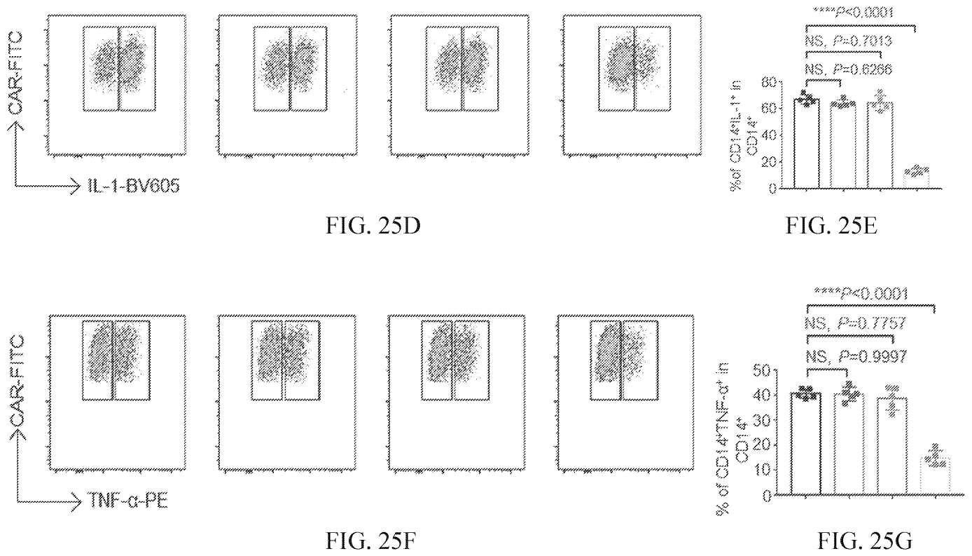

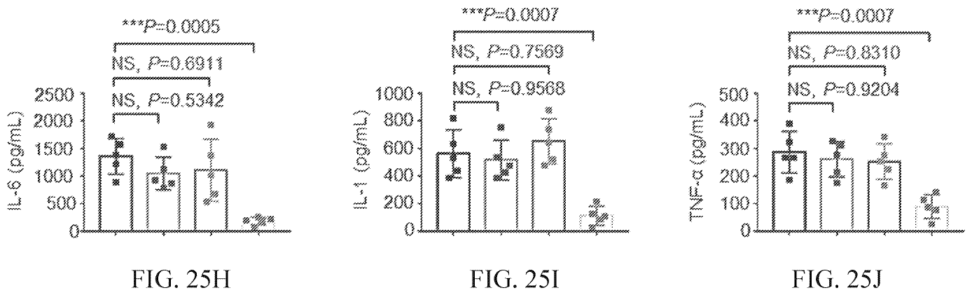

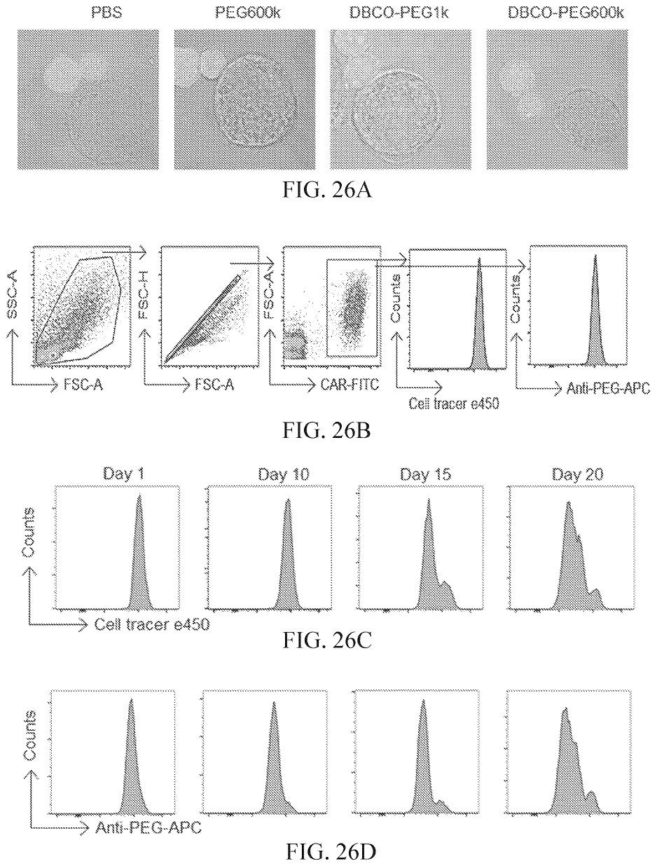

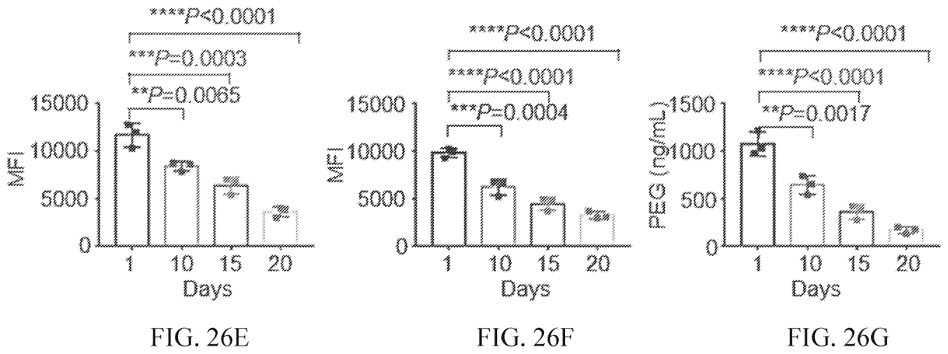

Next, a series of studies was conducted to determine if DBCO-PEG600k conjugation to CAR T cells was responsible for the alleviation of CRS. As before, a humanized CRS mouse model was generated (FIG. 3A). Upon injection of CAR T cells at day 0, a similar dramatic body temperature increase was observed at day 1 (FIG. 3B), indicative of CRS. 10 mg/kg DBCO-PEG1k, unmodified PEG600k and DBCO-PEG600k were injected intravenously into mice on day 1. DBCO-PEG1k is too short to block cell-to-cell contacts, while unmodified PEG600k lacks the DBCO group to conjugate to the surface of CAR T cells and form a polymeric spacer. Mouse body weight, temperature, and blood cytokines were followed over the course of 35 days (FIG. 3c-i). As expected, treatment with DBCO-PEG1k and unmodified PEG600k did not affect weight loss, temperature, and cytokine release compared to a PBS infusion (FIGS. 3B-3I). However, DBCO-PEG600k treatment greatly reversed weight loss, high fever, and cytokine release (FIGS. 3B-3I). Although cytokine levels remained elevated for a longer duration of time in mice treated with DBCO-PEG600k compared to the PBS, DBCO-PEG1k, and PEG600k groups (FIGS. 3D-3I), the peak elevation of cytokine levels was much lower comparatively. In addition to this decrease in peak elevation, all parameters including body weight, body temperature, and cytokine levels recovered to a normal range by day 35 in mice treated with DBCO-PEG600k (FIGS. 3B-3I). The number of tumor cells, CAR T cells, and monocytes in blood was also measured (FIG. 3j-l). In both DBCO-PEG1k- and unmodified PEG600k-treated mice, almost all tumor cells were cleared within 2-3 days after CAR T cell infusion. However, in mice treated with DBCO-PEG600k there was a significant delay in tumor cell clearance, but all tumor cells were eventually cleared at day 35 post-CAR T cell injection (FIG. 3J). Along with the clearance of tumor cells, a rapid increase in the number of CAR T cells and CD14⁺ monocytes was observed following CAR T cell infusion in the PBS, DBCO-PEG1k, and PEG600k groups (FIGS. 3K, 3L). However, in mice treated with DBCO-PEG600k, the expansion of both CAR T cells and CD14⁺ monocytes was greatly reduced (FIGS. 3K, 3L). 5 Moreover, the amount of IL-6⁺, IL-1⁺, and TNF-α⁺ monocytes at day 7 were greatly decreased in mice treated with DBCO-PEG600k compared to those in the PBS, PEG600k, or DBCO-PEG1k-treated mice (FIGS. 25A-25J). Since high levels of CAR T cell and monocyte expansion levels are two well-known signs of life-threatening CRS, these results provide further support of the use of this PEGylation strategy in alleviating CRS-related symptoms.

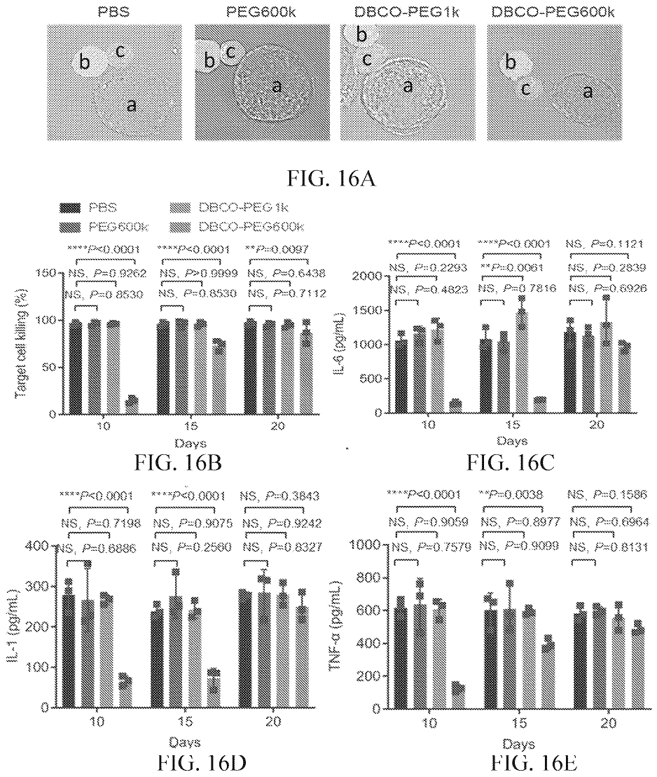

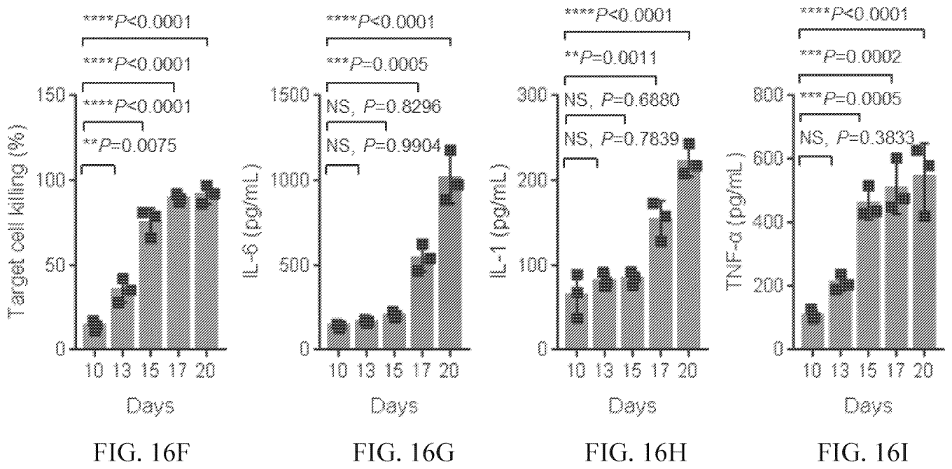

Since treatment with DBCO-PEG600k alleviated symptoms of CRS, and without wishing to be bound by theory, it was hypothesized that certain cell-to-cell interactions between CAR T cells, tumor cells, and monocytes were affected and sought to investigate the mechanism behind this phenomenon. To verify whether the polymeric spacer is present on the surface of azide-labeled CAR T cells and blocks cell-to-cell interactions after a DBCO-PEG600k injection on day 1, CAR T cells were sorted from mouse blood at day 7, and co-cultured them with Raji-Luc-GFP cells and human monocytes for 24 h. CAR T cells from mice treated with DBCO-PEG1k and unmodified PEG600k induced substantial Raji-Luc-GFP cell lysis and cytokine release, whereas CAR T cells isolated from mice treated with DBCO-PEG600k did not induce high cytokine release and target cell killing (FIGS. 3M, 3O, 3P). Confocal microscopy images of CAR T cells from mice treated with DBCO-PEG600k that were co-cultured with tumor cells and monocytes suggested that cell-to-cell interactions were blocked because cell-cell interactions were not visible (FIG. 16A).

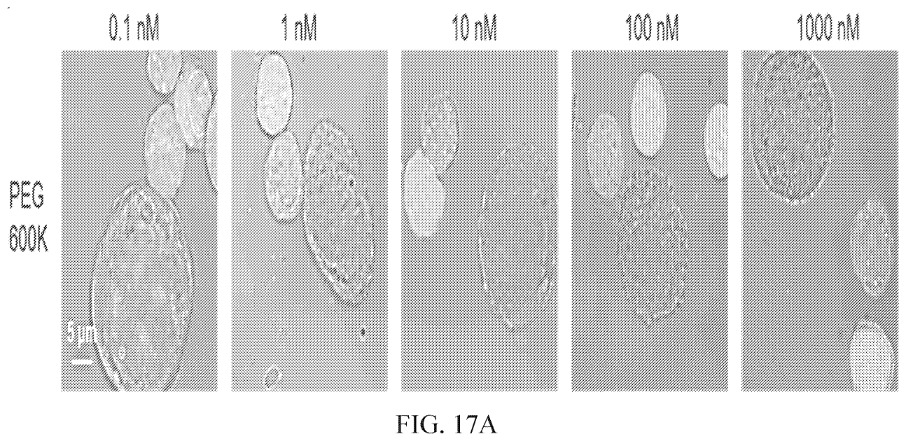

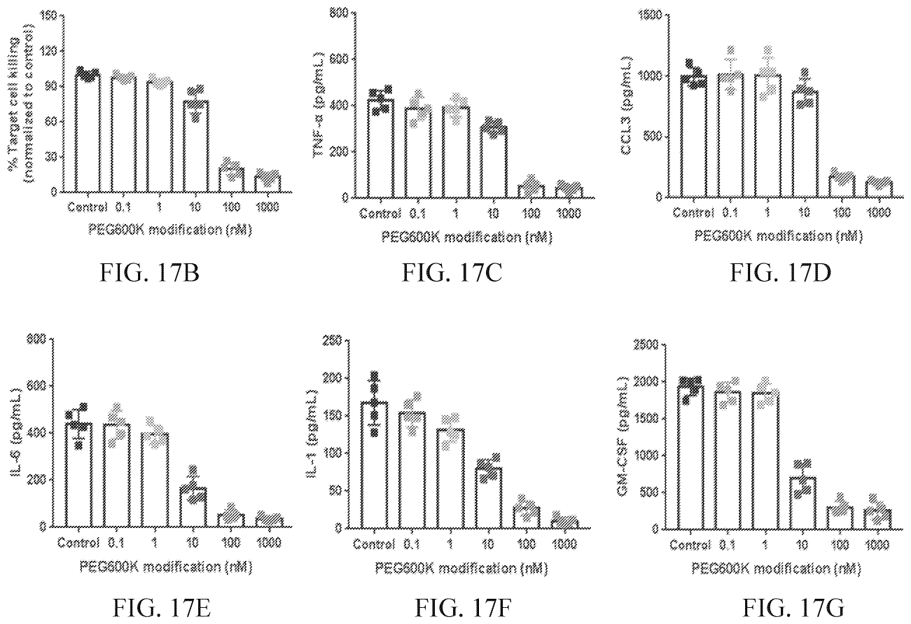

Because tumor cells were eventually cleared, it was possible that expansion of CAR T cells in DBCO-PEG600k-treated mice might have diluted PEG on the surface of CAR T cells enough to restore cell-to-cell interactions and enable tumor cell killing. To demonstrate this, CAR T cells were isolated from DBCO-PEG600k-treated mice at day 10, day 15 and day 20. These cells were co-cultured with both Raji-Luc-GFP cells and monocytes for 24 h. These results showed that CAR T cells collected at day 10 did not induce tumor cell killing and cytokine release (IL-6, IL-1, TNF-α) compared to other groups (FIGS. 16B-16H), indicating that cell-to-cell interactions were still blocked at day 10. However, CAR T cells collected at day 15 and day 20 induced substantial target cell killing and cytokine release (FIGS. 16B, 16C, 16D). It was hypothesized that, as DBCO-PEG600k was gradually diluted, cell-to-cell interactions were gradually restored. Remarkably, at day 15, CAR T cells induced substantial tumor cell killing and release of CAR T cell-associated cytokine TNF-α, but not monocyte-related cytokines (IL-6 and IL-1). Increased levels of both CAR T cell cytokine (TNF-α) and monocyte cytokines (IL-6 and IL-1) were detected at day 20 (FIGS. 16E, 16F), suggesting the potential for restored interactions between CAR T cells, tumor cells, and macrophages. The restoration of CAR T cell-tumor cell interactions (day 15) occurs before the restoration of CAR T cell-monocyte interactions (day 20). This is likely due to the smaller size of the Raji tumor cells (7-10 μm) and T cells (5-8 μm) than monocytes (30-50 μm). As CAR T cells slowly expand, the PEG spacer becomes more diluted on the cell surface, thus restoring interactions with smaller cells (tumor cells) before larger cells (monocytes). The size-dependent sequential cell-to-cell interaction restoration was also confirmed with an in vitro DBCO-PEG600k density experiment (FIG. 17). CAR T cells were cultured in media containing PEG600k at different concentrations (0.1 nM, 1 nM, 10 nM, 100 nM, and 1000 nM) and were co-cultured with monocytes and Raji target cells. Target cell killing and cytokine release assays demonstrated that cell-to-cell interactions were blocked more easily as the PEG600k density was increased (FIG. 17). Notably, in the group with CAR T cells modified with 10 nM DBCO-PEG600k, CAR T cells induced substantial target cell lysis and T cell cytokine release (FIGS. 17C-17E) without inducing cytokine release from monocytes (FIGS. 17F-17H). These results indicate that there is a cell surface density level of PEG600k necessary to block interactions between CAR T cells and macrophages, while leaving CAR T-target cell interactions unaffected. This further supports the hypothesis that when PEGylated CAR T cells slowly expand n vivo, dilution of the PEG spacer restores CAR T cell-tumor cell interactions to allow tumor cell killing in the absence of monocyte over-activation to reduce CRS.

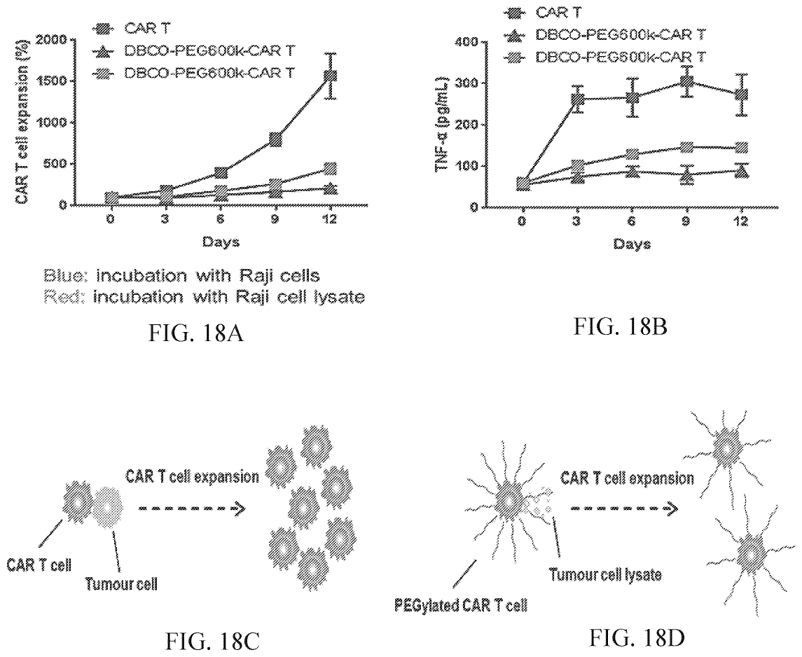

CAR T cell expansion in vivo is highly dependent upon the recognition of tumor cells and tumor antigens. However in the present study, the DBCO-PEG600k modification blocked CAR T cell-tumor cell interactions, and thus tumor cells could not induce rapid expansion of the PEGylated CAR T cells. However, slow expansion of in situ modified DBCO-PEG600k CAR T cells was still observed, so it seems that the expansion of CAR T cells may be induced by tumor antigens released from the lysed tumor cells. To demonstrate this, 1×10⁶ CAR T cells or DBCO-PEG600k-modified CAR T cells were co-cultured with 1×10⁶ Raji tumor cells or cell lysate of 1×10⁶ Raji tumor cells. CAR T cell numbers and TNF-α concentrations in cell culture medium were determined every three days. As expected, tumor cell lysate induced slower expansion of PEGylated CAR T cells and lower TNF-α release compared to CAR T cells that were co-cultured with Raji tumor cells (FIGS. 18C, 18D). These results demonstrate that DBCO-PEG600k-modified CAR T cells slowly expand in response to tumor cell lysate, allowing for slow dilution of PEG on the CAR T cell surface to enable tumor cell killing before the initiation of CAR T cell-induced monocyte activation.

### Example 3: In Situ PEGylation of CAR T Cells Alleviates Neurotoxicity

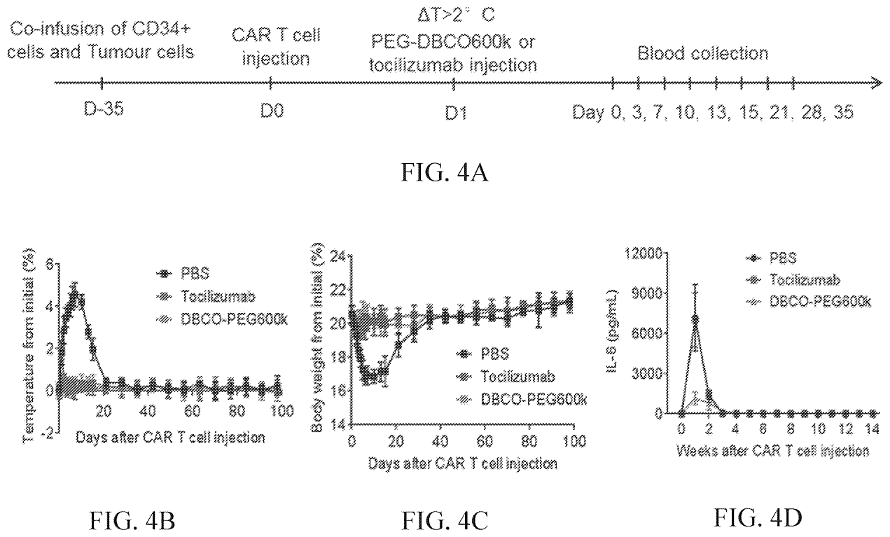

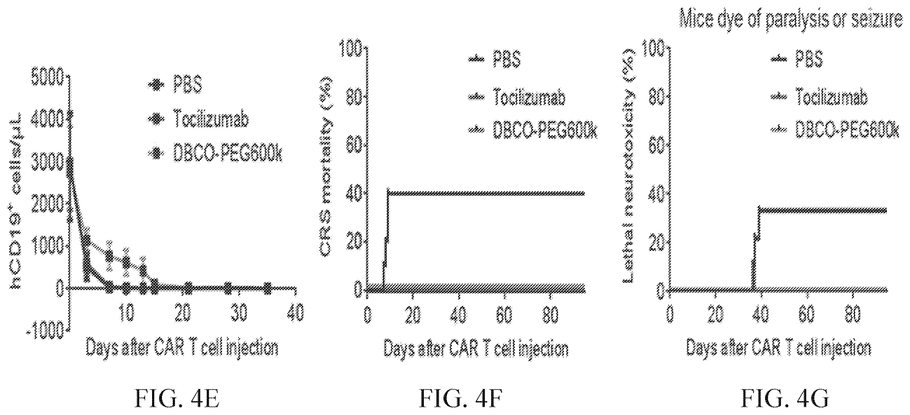

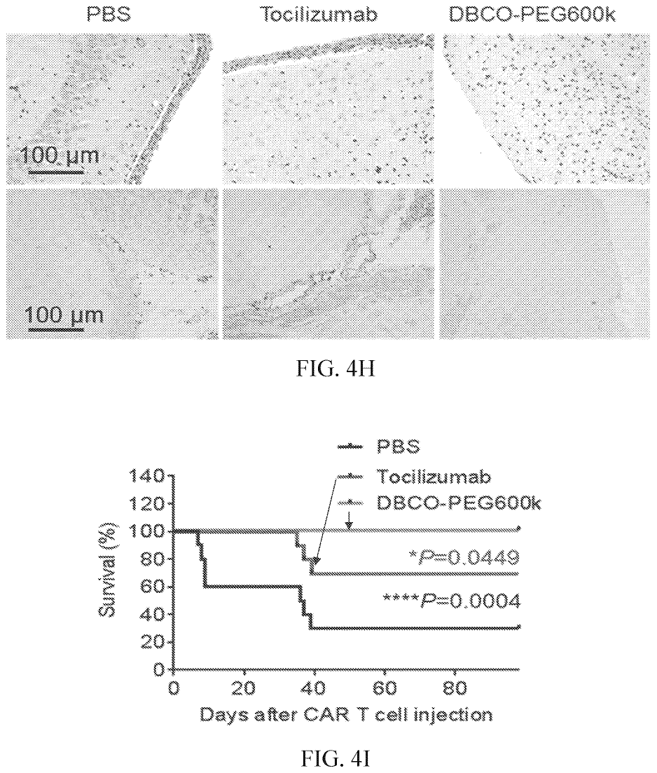

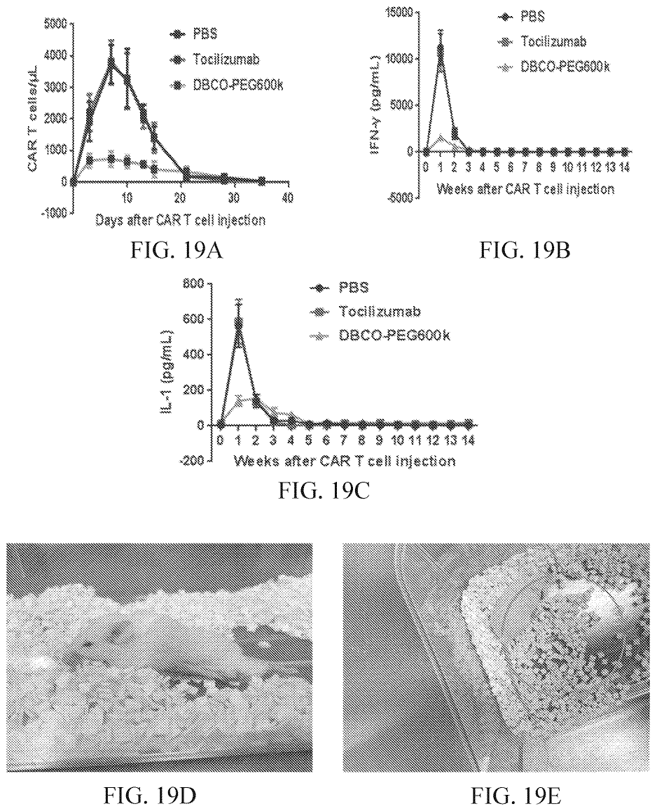

Tocilizumab is often used in the clinic to manage CAR T cell-induced toxicity by blocking the IL-6 receptor. Like DBCO-PEG600k, tocilizumab is administered intravenously once CRS symptoms arise. Although it is effective in ablating some symptoms related to severe CRS, it is not very effective in reversing severe neurotoxicity. Several studies have found that IL-1, released by monocytes, is a major contributor to neurotoxicity. Motivated by the in vitro and in vivo finding that suggests IL-1 levels are decreased in the DBCO-PEG600k-treated group, studies then investigated whether a DBCO-PEG600k injection has advantages over a tocilizumab injection to treat CRS symptoms (FIG. 4A). 24 h after CAR T cell infusion, tumor bearing NSG-SGM3 mice developed high fever (ΔT>2 °C), indicative of CRS. Mice were injected with either tocilizumab (10 mg/kg), DBCO-PEG600k (10 mg/kg), or PBS. CRS-related symptoms such as high fever and weight loss were found to be controlled in both tocilizumab- and DBCO-PEG600k-treated mice (FIGS. 4B, 4C). Though CAR T cell expansion and IFN-γ production occurred over a longer time in DBCO-PEG600k-treated mice compared to tocilizumab-treated mice (FIGS. 19A, 19B), peak CAR T cell expansion levels and IFN-γ production were much lower in DBCO-PEG600k-treated mice compared to mice treated with tocilizumab (FIGS. 19A, 19B). While tocilizumab treatment did not affect IL-6 and IL-1 levels in the blood, DBCO-PEG600k significantly decreased both IL-6 and IL-1 levels (FIG. 4D, FIG. 19C). Importantly, tumor cells were completely cleared by CAR T cells in humanized NSG-SGM3 mice that received either tocilizumab or DBCO-PEG600k by day 35 (FIG. 4E).

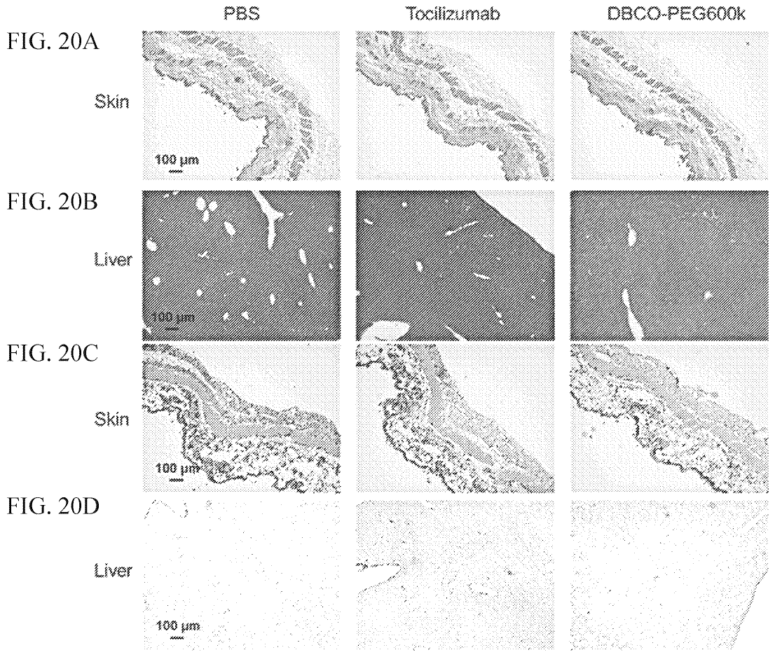

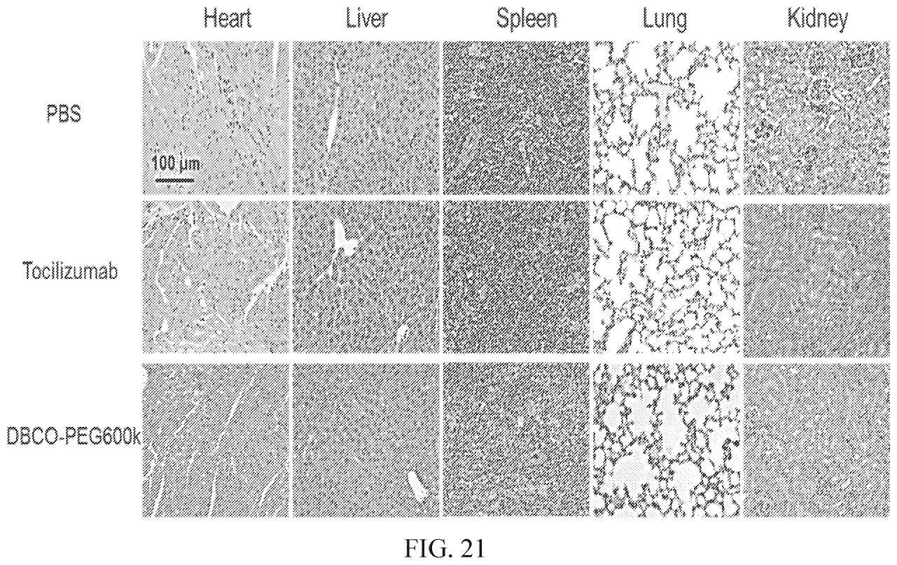

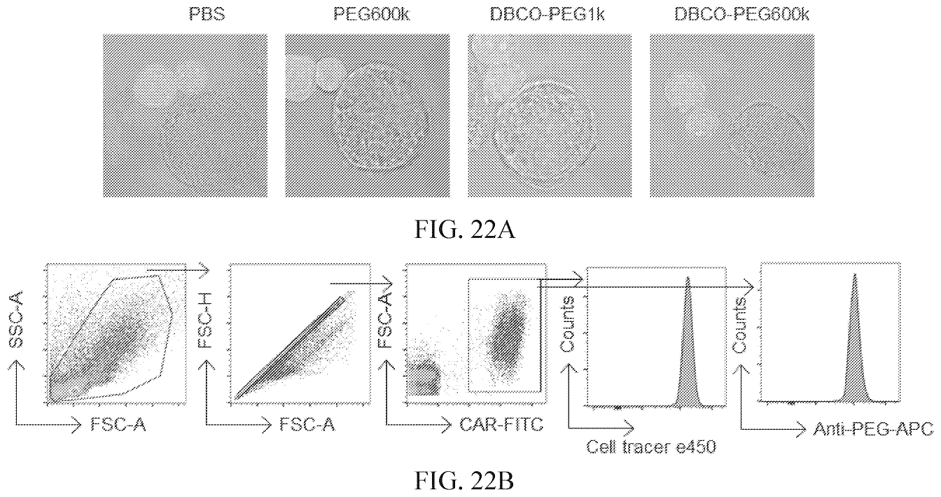

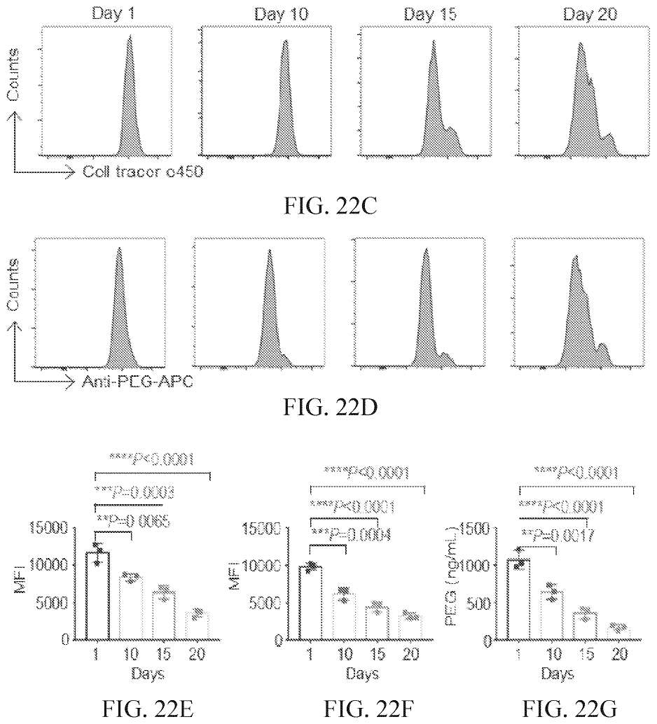

After approximately 35 days post-injection of CAR T cells, tumor-bearing NSG-SGM3 mice that received either PBS or tocilizumab developed sudden paralysis (FIG. 19E) or seizure (FIG. 19F), which are signs of lethal neurological syndrome. However, mice injected with DBCO-PEG600k did not show any signs of paralysis of seizure. This form of delayed neurotoxicity was found to happen only in mice with previous CRS. No signs of X-GVHD in the skin and liver were detected according to postmortem analysis (FIG. 20). Remarkably, both tocilizumab and DBCO-PEG600k protected mice from CRS mortality (FIG. 4F), but only DBCO-PEG600k prevented mice from developing lethal neurotoxicity (FIG. 4G). PBS-treated and tocilizumab-treated mice did show brain meningeal thickening (FIG. 4H, FIG. 21) accompanied by human monocyte infiltration in the subarachnoid space, as determined by immunohistochemistry analysis of human CD68 (FIG. 4H, FIG. 21). Brain meningeal thickening and human monocyte infiltration were not observed in mice treated with DBCO-PEG600k. These data demonstrate that DBCO-PEG600k protected mice from severe neurotoxicity that tocilizumab was not able to protect against. As a result, only DBCO-PEG600k—and not tocilizumab—significantly prolonged mice survival (FIG. 4I).

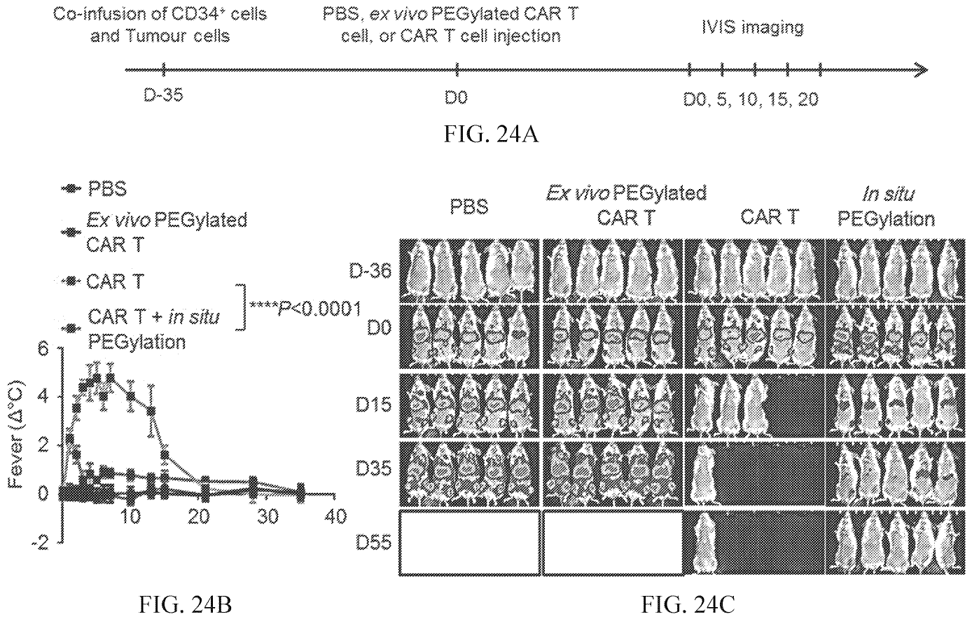

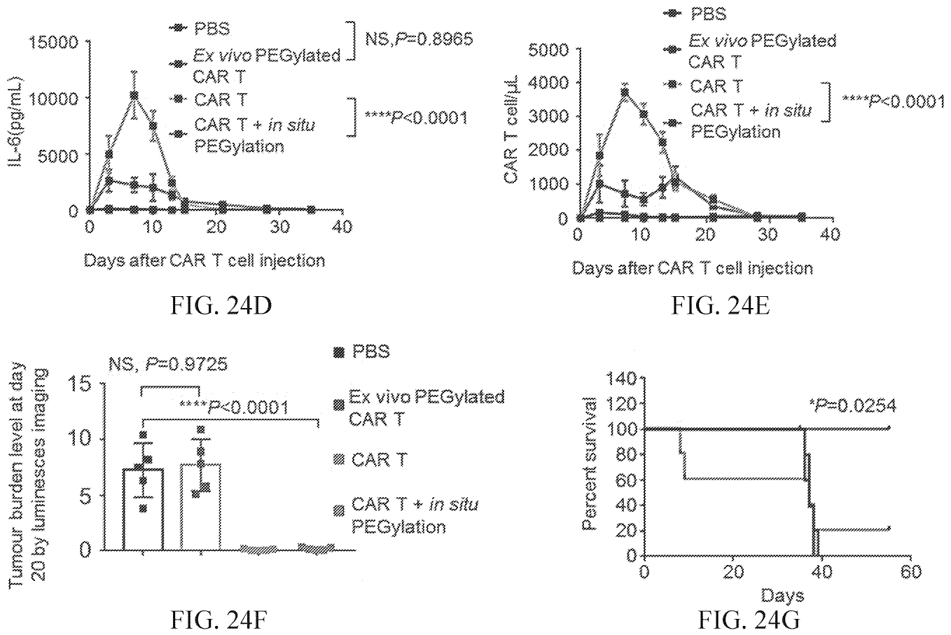

Studies also asked if conjugation of PEG600k to CAR T cells ex vivo and infusion of the cells back into mice could treat CRS and neurotoxicity (FIG. 24A). The Raji tumor model was constructed and the mice were treated with either ex vivo PEGylated CAR T cells or regular CAR T-azide cells at day 0. On day 1, the mice treated with CAR T-azide cells were found to have developed high fever, so DBCO-PEG600k was injected into some of the mice (FIG. 24B). However, mice that received ex vivo PEGylated CAR T cells failed to develop high fever. Tumor growth was monitored using IVIS (FIG. 24C) and blood IL-6 (FIG. 24D) and CAR T cell levels in the blood (FIG. 24E) were documented. Here it was found that ex vivo PEGylated CAR T cells failed to induce IL-6 storm in the mice. However, ex vivo PEGylated CAR T cells did not inhibit tumor growth compared to PBS treatment (FIG. 24F). In contrast, DBCO-PEG600k treatment greatly decreased the peak IL-6 concentration and completely cleared tumor cells in vivo (FIG. 24C). Even though the ex vivo approach did not induce CRS, the fast growth of tumor cells lead to rapid mouse death (FIG. 24G). This is because the surfaces of ex vivo PEGylated CAR T cells were fully coated with PEG, preventing the cells from reaching any tumor cells, thus prohibiting CAR T cell expansion (FIG. 24D). However, with the in situ PEGylation strategy, the initial CAR T cell-induced cancer cell killing led to the release of tumor antigens, which can penetrate between the PEG spacers, while slow expansion of CAR T cells restored the CAR T-tumor cell interactions (FIG. 18). These results demonstrated that only the in situ PEGylation strategy can alleviate CAR T cell-induced CRS and neurotoxicity while not hampering their tumor killing ability.

### Example 4: Tetrazine-Trans-Cyclooctene Reaction for In Situ PEGylation

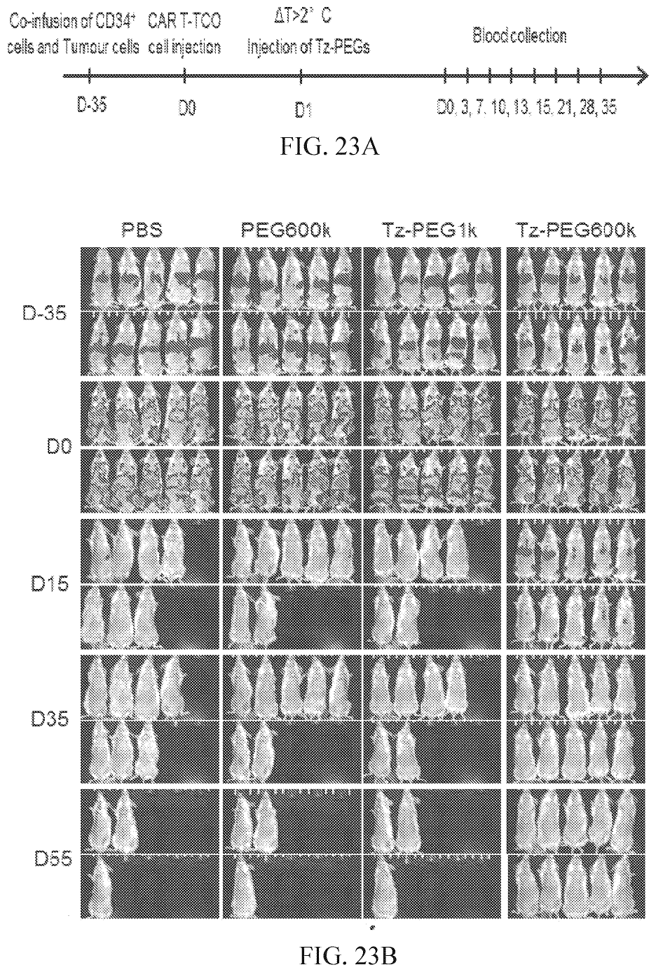

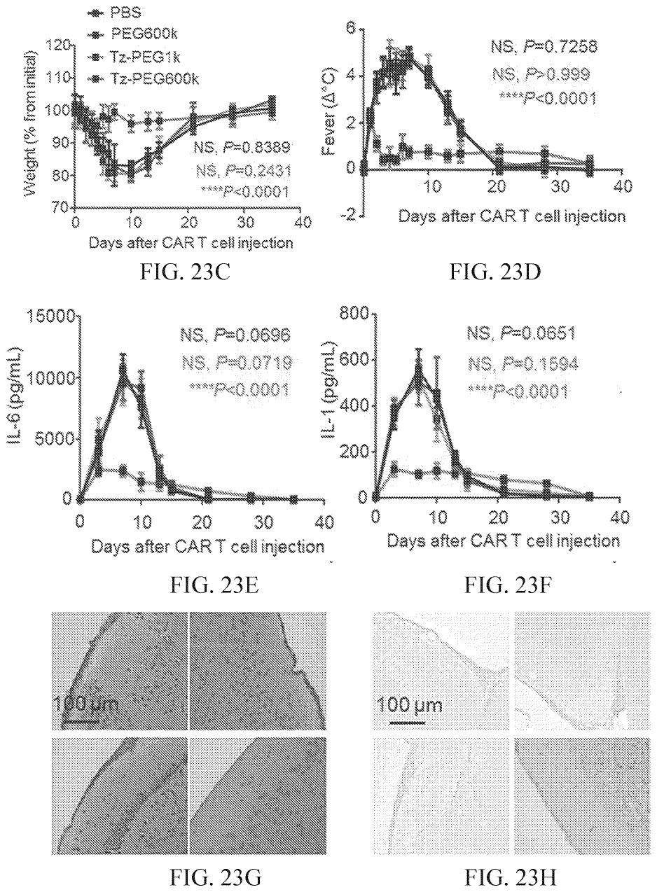

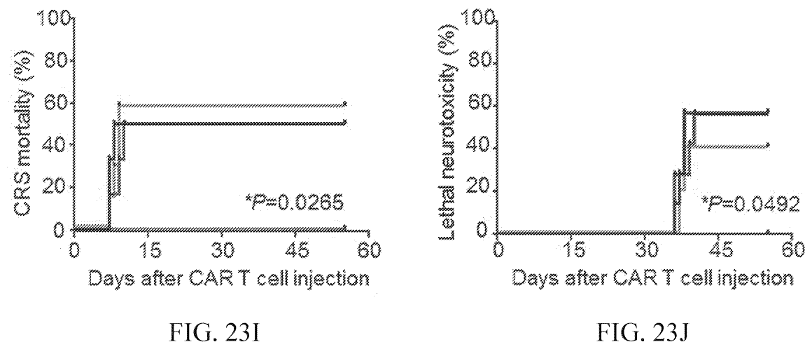

In order to explore if this in situ PEGylation strategy could be generalized to chemistries beyond DBCO-azide, a series of studies then investigated the use of the tetrazine (Tz)-trans-cyclooctene (TCO) reaction for its ability to control CAR T cell-mediated CRS and neurotoxicity (FIGS. 23A-23J).

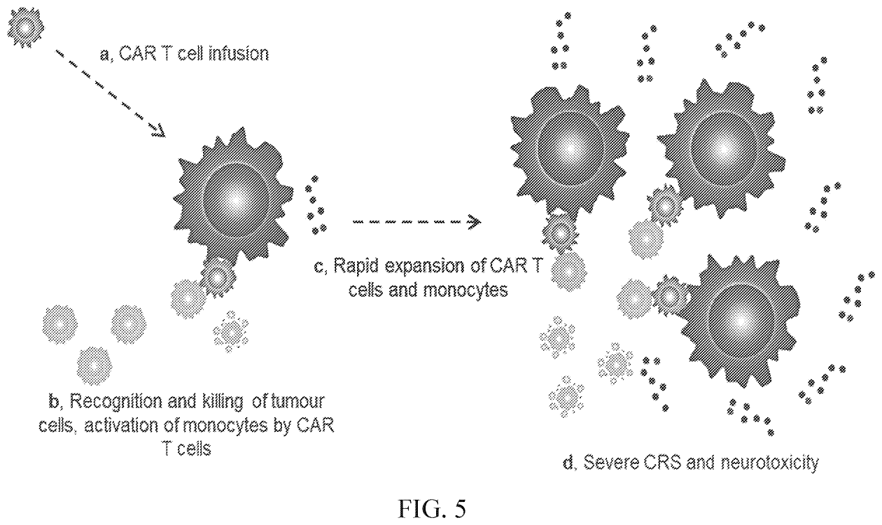

PEG600k, Tz-PEG1k, Tz-PEG600k, or PBS was i.v. infused into the mice. Similar to what was seen in the DBCO-azide click chemistry-mediated in situ PEG modification, treatment with Tz-PEG1k and unmodified PEG600k did not affect tumour growth, weight loss, high fever, and cytokine release compared to PBS infusion (FIGS. 23B-23F). However, Tz-PEG600k treatment greatly reversed weight loss, high fever, and cytokine release (FIGS. 5C-5F). Moreover, tumour cells in the Tz-PEG600k treated mice were cleared at around day 35 post-CAR T cell injection (FIG. 5B). Further extending the duration of the experiment, a high proportion of mice in PBS-, PEG600k-, and Tz-PEG1k-treated groups showed signs of neurological toxicities. However, the mice in Tz-PEG600k treated group did not show such neurological toxicity (FIGS. 5G-5H). Moreover, Tz-PEG600k can greatly decrease CRS mortality and lethal neurotoxicity (FIGS. 5I-5J).

Based on these results from the DBCO-based click chemistry and the Tz-TCO chemistry, in situ PEGylation of CAR T cells can act to alleviate CRS and neurotoxicity in vivo.

### Example 5: Selected Discussion

CAR T cell immunotherapy has revolutionized cancer therapy in the clinic. However, cytokine release syndrome (CRS) and neurotoxicity restrict the broader application of this therapy. This toxicity is a result of intensive tumor cell lysis, rapid CAR T cell expansion and monocyte over-activation. The studies of the present disclosure show that in situ PEGylation of CAR T cells can greatly decrease interactions between CAR T cells, tumor cells, and monocytes, which overall decrease intensive tumor cell lysis and monocyte over-activation by CAR T cells. Over time, CAR T cells slowly expand and DBCO-PEG600k becomes diluted, gradually restoring cell-to-cell interactions. Herein, the disclosed results demonstrate that CAR T cell-tumor cell interactions were restored earlier than CAR T cell-monocyte interactions because of the relative smaller size of B cell lymphoma cells compared to monocytes. This thus creates a therapeutic window for tumor cell killing without monocyte over-activation. In this way, CAR T cells completely clear tumor cells but do not induce severe CRS. A benefit of this system is that the polymeric spacer can be added on an as-needed basis after CAR T cells have been administered—so that the tumor killing ability of CAR T cells is not dampened unless the patient presents symptoms of severe CRS. Furthermore, while both PEGylated CAR T cells and tocilizumab were able to protect against CRS, only PEGylated CAR T cells—and not tocilizumab—were able to protect against severe neurotoxicity. Without wishing to be bound by theory, these results suggest that in situ PEGylation of CAR T cells can be used as a safer CAR T cell therapy.

### ENUMERATED EMBODIMENTS

The following enumerated embodiments are provided, the numbering of which is not to be construed as designating levels of importance.

Embodiment 1 provides a modified immune cell or precursor cell thereof comprising a chimeric antigen receptor (CAR) and one or more surface glycans, wherein at least one of the surface glycans comprises a polysaccharide of formula (I):

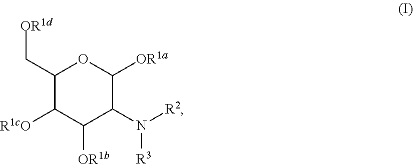

wherein:
R^{1a}, R^{1b}, R^{1c}, and R^{1d} are each independently selected from the group consisting of H, optionally substituted C₁-C₆ alkyl, optionally substituted C₂-C₆ heteroalkyl, optionally substituted C₃-C₈ cycloalkyl, optionally substituted phenyl, optionally substituted benzyl, optionally substituted C₂-C₉ heterocyclyl, C(═O)OR^{a}, C(═O)R^{a}, C(═O)N(R^{a})(R^{b}), S(═O)₂R^{a}, S(═O)₂N(R^{a})(R^{b}), a monosaccharide, a terminal polysaccharide, and a polysaccharide covalently conjugated to a CAR-T cell surface protein,
   wherein one selected from R^{1a}, R^{1b}, R^{1c}, and R^{1d} is the polysaccharide covalently conjugated to the surface protein;
R² is -L¹—Y—L²—Z;
R³ is selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
R⁴ is selected from the group consisting of optionally substituted C₁-C₆ alkyl, C₂-C₆ heteroalkyl, optionally substituted C₃-C₈ cycloalkyl, optionally substituted phenyl, optionally substituted benzyl, optionally substituted C₂-C₉ heterocyclyl, halogen, OR^{a}, N(R^{a})(R^{b}), SR^{a}, CN, and NO₂,
   wherein two adjacent R⁴ substituents may combine with the atoms to which they are bound to form an optionally substituted phenyl, optionally substituted C₃-C₈ cycloalkyl, or optionally substituted C₂-C₉ heterocyclyl;
R⁵ is selected from the group consisting of H and optionally substituted C₁-C₆ alkyl;
R⁶ is selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
L¹ is selected from the group consisting of —C(═O)(optionally substituted C₁-C₆ alkylene)-*, —C(═O)(optionally substituted C₁-C₆ heteroalkylene)-*, -(optionally substituted C₁-C₆ alkylene)-*, and -(optionally substituted C₁-C₆ heteroalkylene)-*;
L² is selected from the group consisting of **—C(═O)(optionally substituted C₁-C₁₂ alkylene)C(═O)O—, **—C(═O)(optionally substituted C₂-C₁₂ heteroalkylene)C(═O)O—, **—C(═O)(optionally substituted C₁-C₁₂ alkylene)O—, **—C(═O)(optionally substituted C₂-C₁₂ heteroalkylene)O—, **-(optionally substituted C₁-C₁₂ alkylene)C(═O)O—, **-(optionally substituted C₂-C₁₂ heteroalkylene)C(═O)O—, **-(optionally substituted C₁-C₁₂ alkylene)O—, and **-(optionally substituted C₂-C₁₂ heteroalkylene)C(═O)O—;
Y is selected from the group consisting of

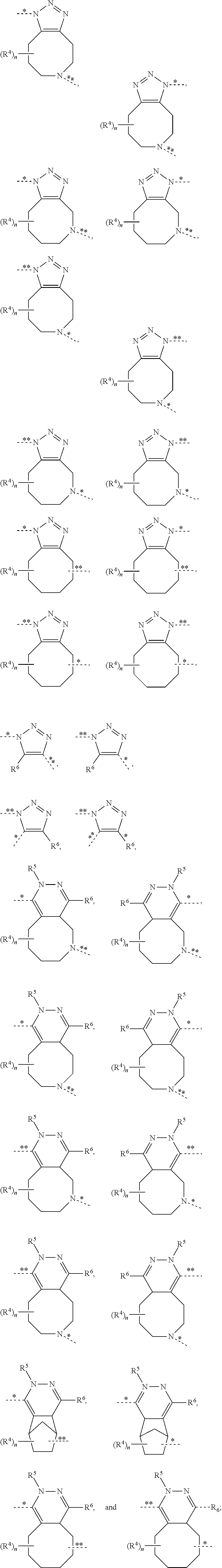

Z is selected from the group consisting of an optionally substituted heteroalkyl group, a dendrimer, a microparticle, a nanoparticle, an alignate, a biomolecule, and a modified red blood cell,
   wherein the dendrimer, microparticle, nanoparticle, alignate, biomolecule, or modified red blood cell can be covalently linked to L² by an optionally substituted heteroalkylene, and
   wherein each heteroalkyl or heteroalkylene has a molecular weight ranging from about 1 kDa to about 600 kDa;
each occurrence of R and R is independently selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
n is an integer ranging from 0 to 11;
* indicates a bond between L¹ and Y; and
** indicates a bond between Y and L².

Embodiment 2 provides the modified immune cell or precursor cell thereof of embodiment 1, wherein each occurrence of optionally substituted alkyl, optionally substituted heteroalkyl, optionally substituted cycloalkyl, optionally substituted alkoxy, optionally substituted phenyl, optionally substituted benzyl, optionally substituted heterocyclyl, optionally substituted alkylene, and optionally substituted heteroalkylene, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₉ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

Embodiment 3 provides the modified immune cell or precursor cell thereof of embodiment 1 or 2, wherein the compound of formula (I) is a compound of formula (Ia):

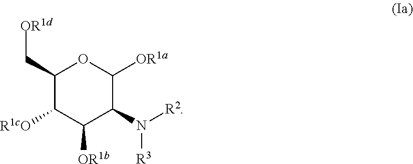

Embodiment 4 provides the CAR-T cell of any one of embodiments 1-3, wherein one of the following applies:
(a) R^{1a} is the polysaccharide covalently conjugated to the surface protein, and each of R^{1b}, R^{1c}, and R^{1d} are C(═O)CH₃ or H;
(b) R^{1b} is the polysaccharide covalently conjugated to the surface protein, and each of R^{1a}, R^{1c}, and R^{1d} are C(═O)CH₃ or H;
(c) R^{1c} is the polysaccharide covalently conjugated to the surface protein, and each of R^{1a}, R^{1b}, and R^{1d} are C(═O)CH₃ or H;
(d) R^{1d} is the polysaccharide covalently conjugated to the surface protein, and each of R^{1a}, R^{1b}, and Ric are C(═O)CH₃ or H;
(e) R^{1a} is the polysaccharide covalently conjugated to the surface protein, one of R^{1b}, R^{1c}, and R^{1d} is a monosaccharide or a terminal polysaccharide, and two of R^{1b}, R^{1c}, and R^{1d} are C(═O)CH₃ or H;
(f) R^{1b} is the polysaccharide covalently conjugated to the surface protein, one of R^{1a}, R^{1c}, and R^{1d} is a monosaccharide or a terminal polysaccharide, and two of R^{1a}, R^{1c}, and R^{1d} are C(═O)CH₃ or H;
(g) R^{1c} is the polysaccharide covalently conjugated to the surface protein, one of R^{1a}, R^{1b}, and R^{1d} is a monosaccharide or a terminal polysaccharide, and two of R^{1a}, R^{1b}, and R^{1d} are C(═O)CH₃ or H; and
(h) R^{1d} is the polysaccharide covalently conjugated to the surface protein, one of R^{1a}, R^{1b}, and R^{1c} is a monosaccharide or a terminal polysaccharide, and two of R^{1a}, R^{1b}, and R^{1c} are C(═O)CH₃ or H.

Embodiment 5 provides the modified immune cell or precursor cell thereof of any one of embodiments 1-4, wherein R³ is H.

Embodiment 6 provides the modified immune cell or precursor cell thereof of any one of embodiments 1-5, wherein each occurrence of R⁴ is H.

Embodiment 7 provides the modified immune cell or precursor cell thereof of any one of embodiments 1-6, wherein R⁵ is H.

Embodiment 8 provides the modified immune cell or precursor cell thereof of any one 5 of embodiments 1-7, wherein R⁶ is H.

Embodiment 9 provides the modified immune cell or precursor cell thereof of any one of embodiments 1-8, wherein L¹ is —C(═O)CH₂—*.

Embodiment 10 provides the modified immune cell or precursor cell thereof of any one of embodiments 1-9, wherein L² is **—C(═O)(1,5-pentylene)C(═O)—.

Embodiment 11 provides the modified immune cell or precursor cell thereof of any one of embodiments 1-5 and 7-10, wherein Y is

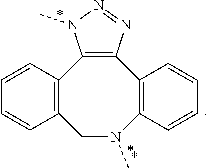

Embodiment 12 provides the modified immune cell or precursor cell thereof of any one of embodiments 1-11, wherein the optionally substituted heteroalkyl in Z is selected from the group consisting of a polyethylene glycol (PEG) polymer, a polyamidoamine (PAMAM) polymer, a polyethyleneimine (PEI) polymer, a polymethyl methacrylate (PMMA) polymer, a poly(N-isopropyleneimine) (PPI) polymer, and a polyvinyl alcohol (PVA) polymer.

Embodiment 13 provides the modified immune cell or precursor cell thereof of embodiment 12, wherein the PEG polymer has the following formula:

—(CH₂CH₂O)ₒ—H,
wherein o is an integer ranging from 1 to 14,000.

Embodiment 14 provides the modified immune cell or precursor cell thereof of any one of embodiments 1-13, wherein the heteroalkyl or heteroalkylene in Z has a molecular weight selected from the group consisting of about 1 kDa, about 5 kDa, about 10 kDa, about 100 kDa, and about 600 kDa.

Embodiment 15 provides the modified immune cell or precursor cell thereof of any one of embodiments 1-5 and 7-14, wherein R² is selected from the group consisting of:

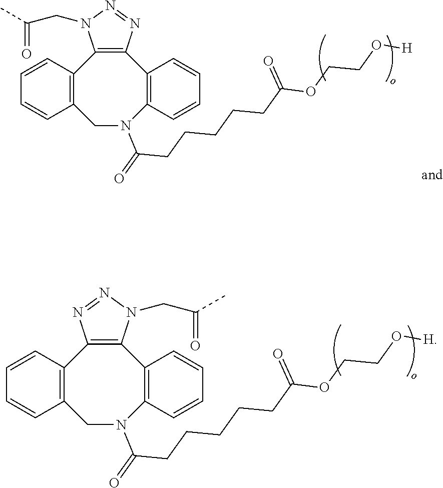

Embodiment 16 provides the modified immune cell or precursor thereof of any one of embodiments 1-15, wherein the surface glycan blocks cell-cell interactions by stearic hindrance.

Embodiment 17 provides the modified immune cell or precursor cell thereof of any one of embodiments 1-16, wherein the chimeric antigen receptor comprises an antigen-binding domain capable of binding a tumor-associated antigen.

Embodiment 18 provides the modified immune cell or precursor cell thereof of any one of embodiments 1-17, wherein the modified immune cell or precursor cell thereof is selected from the group consisting of an αβ T cell, a δϵ T cell, a CD8 T cell, a CD4 helper T cell, a CD4 regulatory T cell, an NK T cell, an NK cell, and any combination thereof.

Embodiment 19 provides the modified immune cell or precursor cell thereof of any one of embodiments 1-18, wherein the modified immune cell or precursor thereof is a T cell.

Embodiment 20 provides a method of preparing a modified immune cell or precursor cell thereof, the method comprising:
(a) contacting an immune cell or precursor cell thereof comprising a chimeric antigen receptor and further comprising one or more surface glycans, wherein at least one of the surface glycans comprises a polysaccharide of formula (It):

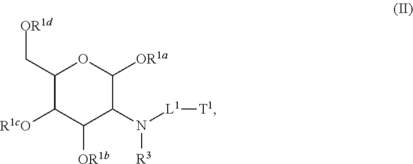

wherein:
   R^{1a}, R^{1b}, R^{1c}, and R^{1d} are each independently selected from the group consisting of H, optionally substituted C₁-C₆ alkyl, optionally substituted C₂-C₆ heteroalkyl, optionally substituted C₃-C₈ cycloalkyl, optionally substituted phenyl, optionally substituted benzyl, optionally substituted C₂-C₉ heterocyclyl, C(═O)OR^{a}, C(═O)R^{a}, C(═O)N(R^{a})(R^{b}), S(═O)₂R^{a}, S(═O)₂N(R^{a})(R^{b}), a monosaccharide, a terminal polysaccharide, and a polysaccharide covalently conjugated to a CAR-T cell surface protein,
   wherein one selected from R^{1a}, R^{1b}, R^{1c}, and R^{1d} is the polysaccharide covalently conjugated to the surface protein;
   R³ is selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
      R⁴ is selected from the group consisting of optionally substituted C₁-C₆ alkyl, C₂-C₆ heteroalkyl, optionally substituted C₃-C₈ cycloalkyl, optionally substituted phenyl, optionally substituted benzyl, optionally substituted C₂-C₉ heterocyclyl, halogen, OR^{a}, N(R^{a})(R^{b}), SR^{a}, CN, and NO₂,
   wherein two adjacent R⁴ substituents may combine with the atoms to which they are bound to form an optionally substituted phenyl, optionally substituted C₃-C₈ cycloalkyl, or optionally substituted C₂-C₉ heterocyclyl;
   R⁵ is selected from the group consisting of H and optionally substituted C₁-C₆ alkyl;
   R⁶ is selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
   L¹ is selected from the group consisting of —C(═O)(optionally substituted C₁-C₆ alkylene)-*, —C(═O)(optionally substituted C₁-C₆ heteroalkylene)-*, -(optionally substituted C₁-C₆ alkylene)-*, and -(optionally substituted C₁-C₆ heteroalkylene)-*;
   T¹ is selected from the group consisting of N₃

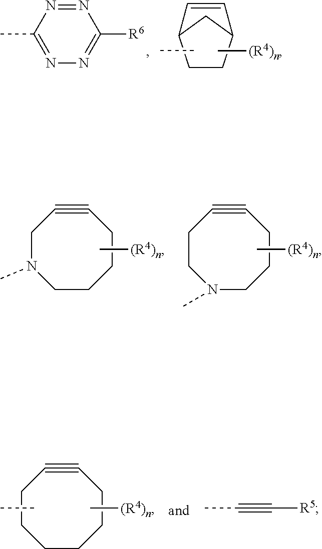

each occurrence of R^{a} and R^{b} is independently selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
n is an integer ranging from 0 to 11; and
(b) a compound of formula (III):

T²-L²-Z (III),
wherein:
   T² is selected from the group consisting of —N₃,

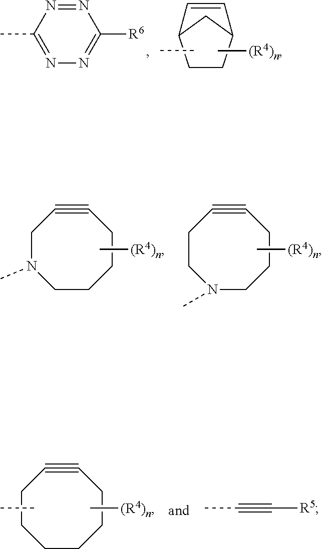

and
L² is selected from the group consisting of **—C(═O)(optionally substituted C₁-C₁₂ alkylene)C(═O)O—, **—C(═O)(optionally substituted C₂-C₁₂ heteroalkylene)C(═O)O—, **—C(═O)(optionally substituted C₁-C₁₂ alkylene)O—, **—C(═O)(optionally substituted C₂-C₁₂ heteroalkylene)O—, **-(optionally substituted C₁-C₁₂ alkylene)C(═O)O—, **-(optionally substituted C₂-C₁₂ heteroalkylene)C(═O)O—, **-(optionally substituted C₁-C₁₂ alkylene)O—, and **-(optionally substituted C₂-C₁₂ heteroalkylene)C(═O)O—;
Z is selected from the group consisting of an optionally substituted heteroalkyl group, a dendrimer, a microparticle, a nanoparticle, an alignate, a biomolecule, and a modified red blood cell,
   wherein the dendrimer, microparticle, nanoparticle, alignate, biomolecule, or modified red blood cell can be covalently linked to L² by an optionally substituted heteroalkylene, and
   wherein each heteroalkyl or heteroalkylene has a molecular weight ranging from about 1 kDa to about 600 kDa.

Embodiment 21 provides the method of embodiment 20, wherein one of the following applies:
(a) T¹ is selected from the group consisting of —N₃ and

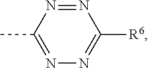

and T² is selected from the group consisting of

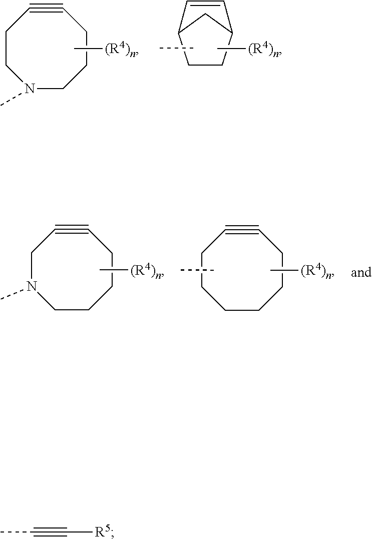

(b) T¹ is selected from the group consisting of

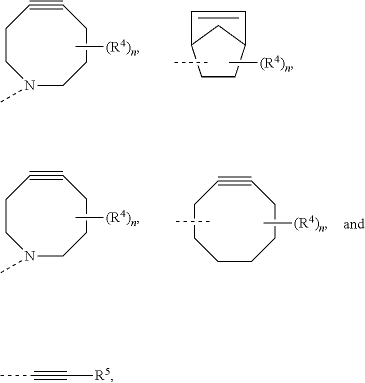

and T² is selected from the group consisting of —N₃ and

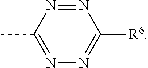

Embodiment 22 provides the method of embodiment 20 or 21, wherein the compound of formula (II) is a compound of formula (IIa):

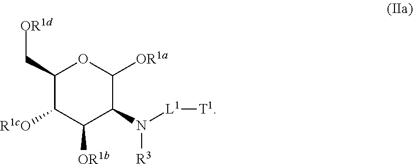

Embodiment 23 provides the method of any one of embodiments 20-22, wherein one of the following applies:
(a) R^{1a} is the polysaccharide covalently conjugated to the surface protein, and each of R^{1b}, R^{1c}, and R^{1d} are C(═O)CH₃ or H;
(b) R^{1b} is the polysaccharide covalently conjugated to the surface protein, and each of R^{1a}, R^{1c}, and R^{1d} are C(═O)CH₃ or H;
(c) R^{1c} is the polysaccharide covalently conjugated to the surface protein, and each of R^{1a}, R^{1b}, and R^{1d} are C(═O)CH₃ or H;
(d) R^{1d} is the polysaccharide covalently conjugated to the surface protein, and each of R^{1a}, R^{1b}, and Ric are C(═O)CH₃ or H;
(e) R^{1a} is the polysaccharide covalently conjugated to the surface protein, one of R^{1b}, R^{1c}, and R^{1d} is a monosaccharide or a terminal polysaccharide. and two of R^{1b}, R^{1c}, and R^{1d} are C(═O)CH₃ or H;
(f) R^{1b} is the polysaccharide covalently conjugated to the surface protein, one of R^{1a}, R^{1c}, and R^{1d} is a monosaccharide or a terminal polysaccharide, and two of R^{1a}, R^{1c}, and R^{1d} are C(═O)CH₃ or H;
(g) R^{1c} is the polysaccharide covalently conjugated to the surface protein, one of R^{1a}, R^{1b}, and R^{1d} is a monosaccharide or a terminal polysaccharide, and two of R^{1a}, R^{1b}, and R^{1d} are C(═O)CH₃ or H; and
(h) R^{1d} is the polysaccharide covalently conjugated to the surface protein, one of R^{1a}, R^{1b}, and R^{1c} is a monosaccharide or a terminal polysaccharide, and two of R^{1a}, R^{1b}, and R^{1c} are C(═O)CH₃ or H.

Embodiment 24 provides the method of any one of embodiments 20-23, wherein R³ is H.

Embodiment 25 provides the method of any one of embodiments 20-24, wherein each occurrence of R⁴ is H.

Embodiment 26 provides the method of any one of embodiments 20-25, wherein each occurrence of R⁵ is H.

Embodiment 27 provides the method of any one of embodiments 20-26, wherein each occurrence of R⁶ is H.

Embodiment 28 provides the method of any one of embodiments 20-27, wherein L¹ is —C(═O)CH₂—*.

Embodiment 29 provides the method of any one of embodiments 20-28, wherein L² is **—C(═O)(1,5-pentylene)C(═O)—.

Embodiment 30 provides the method of any one of embodiments 20-29, wherein the optionally substituted heteroalkyl in Z is selected from the group consisting of a polyethylene glycol (PEG) polymer, a polyamidoamine (PAMAM) polymer, a polyethyleneimine (PEI) polymer, a polymethyl methacrylate (PMMA) polymer, a poly(N-isopropyleneimine) (PPI) polymer, and a polyvinyl alcohol (PVA) polymer.

Embodiment 31 provides the method of embodiment 30, wherein the PEG polymer has the following formula:

—(CH₂CH₂O)ₒ—H,

wherein o is an integer ranging from 1 to 14,000.

Embodiment 32 provides the method of any one of embodiments 20-31, wherein Z has a molecular weight selected from the group consisting of about 1 kDa, about 5 kDa, about 10 kDa, about 100 kDa, and about 600 kDa.

Embodiment 33 provides the method of any one of embodiments 20-32, wherein T¹ is N₃.

Embodiment 34 provides the method of any one of embodiments 20-33, wherein T² is

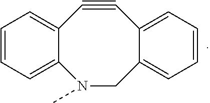

Embodiment 35 provides the method of any one of embodiments 20-34, wherein the compound of formula (III) is:

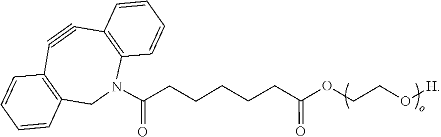

Embodiment 36 provides the method of any one of embodiments 20-35, wherein the chimeric antigen receptor comprises antigen binding domain capable of binding a tumor-associated antigen.

Embodiment 37 provides the method of any one of embodiments 20-36, wherein the modified immune cell or precursor cell thereof is selected from the group consisting of an αβ T cell, a γδ T cell, a CD8 T cell, a CD4 helper T cell, a CD4 regulatory T cell, an NK T cell, an NK cell, and any combination thereof.

Embodiment 38 provides the method of any one of embodiments 20-37, wherein the modified immune cell or precursor thereof is a T cell.

Embodiment 39 provides a method for reducing the severity of a toxicity in a subject caused by administration of a chimeric antigen receptor (CAR) expressing T cell to the subject, said method comprising:
a. labeling the CAR expressing T cell with an azido glycan and/or trans-cyclooctene group and/or tetrazine group, and
b. conjugating a spacer molecule to the azido glycan and/or trans-cyclooctene group and/or tetrazine group, thereby producing a labeled CAR T cell;
   wherein the conjugated spacer molecule reduces the interaction of the CAR T cell with endogenous immune cells, thereby reducing the severity of the toxicity.
      In certain embodiments, the CAR expressing T cell is labeled with an azido glycan and the spacer molecule comprises a dibenzocyclooctyne (DBCO) group. In certain embodiments, the CAR expressing T cell is labeled with a trans-cyclooctene group and the spacer molecule comprises a tetrazine group. In certain embodiments, the CAR expressing T cell is labeled with a tetrazine group and the spacer molecule comprises a trans-cyclooctene group.

Embodiment 40 provides the method of embodiment 39, wherein the spacer molecule is selected from the group consisting of an optionally substituted heteroalkyl group, a dendrimer, a microparticle, a nanoparticle, an alginate, a biomolecule, and a modified red blood cell, wherein the dendrimer, microparticle, nanoparticle, alginate, biomolecule, or modified red blood cell can be covalently linked to the T cell by an optionally substituted heteroalkylene, and wherein each heteroalkyl or heteroalkylene has a molecular weight ranging from about 1 kDa to about 600 kDa.

Embodiment 41 provides the method of embodiment 40, wherein the optionally substituted heteroalkyl is selected from the group consisting of a polyethylene glycol (PEG) polymer, a polyamidoamine (PAMAM) polymer, a polyethyleneimine (PEI) polymer, a polymethyl methacrylate (PMMA) polymer, a poly(N-isopropyleneimine) (PPI) polymer, and a polyvinyl alcohol (PVA) polymer.

Embodiment 42 provides the method of embodiment 41, wherein the PEG polymer has the following formula:

—(CH₂CH₂O)ₒ—H,

wherein o is an integer ranging from 1 to 14,000.

Embodiment 43 provides the method of any one of embodiments 40-42, wherein the heteroalkyl or heteroalkylene has a molecular weight selected from the group consisting of about 1 kDa, about 5 kDa, about 10 kDa, about 100 kDa, and about 600 kDa.

Embodiment 44 provides the method of any one of embodiments 39-43, wherein the labeling of the CAR T cell with an azido glycan and/or trans-cyclooctene group and/or tetrazine group occurs ex vivo from the subject.

Embodiment 45 provides the method of any one of embodiments 39-44, wherein the spacer molecule is a compound of formula (III).

Embodiment 46 provides the method of any one of embodiments 39-45, wherein an effective amount of the spacer molecule is administered to the subject such that the conjugation of the space molecule occurs in vivo in the subject.

Embodiment 47 provides the method of any one of embodiments 39-46, wherein the conjugation of the spacer molecule occurs after administering the CAR T cell to the subject.

Embodiment 48 provides the method of any one of embodiments 39-47, wherein the spacer molecule administration occurs after the administration of the CAR T cell to the subject.

Embodiment 49 provides the method of any one of embodiments 39-48, wherein the azido glycan is N-azidoacetylmannosamine-tetraacylated (Ac4ManNAz).

Embodiment 50 provides the method of any one of embodiments 41-43, wherein the PEG is PEG 600 k.

Embodiment 51 provides the method of any one of embodiments 39-50, wherein the reduction of the interaction of the CAR T cell with endogenous immune cells occurs by steric hindrance.

Embodiment 52 provides the method of any one of embodiments 39-51, wherein the toxicity is selected from the group consisting of cytokine release syndrome (CRS), CAR-T cell-related encephalopathy syndrome (CRES), cytokine release encephalopathy syndrome, immune effector cell associated neurotoxicity syndrome (ICANS), hemophagocytic lymphohistiocytosis/macrophage activation syndrome (HLH/MAS), and any combination thereof.

Embodiment 53 provides the method of any one of embodiments 39-52, wherein the effective amount of the spacer molecule is administered to the subject when the subject presents at least one symptom associated with the toxicity.

Embodiment 54 provides the method of embodiment 53, wherein the symptom associated with the toxicity is selected from the group consisting of high fever, rigors, malaise, fatigue, myalgia, nausea, anorexia, tachycardia/hypotension, hypotension, headache, aphasia, disorientation, lethargy, capillary leak, cardiac dysfunction, renal impairment, hepatic failure, and disseminated intravascular coagulation, elevated IL-6 levels, elevated IL-5 levels, elevated IL-13 levels, elevated IL-10 levels, elevated interferon-gamma (IFNγ) levels dermatitis, tachycardia, hypotension, headache, nausea, aphasia, disorientation, lethargy, and any combination thereof.

Embodiment 55 provides a method of treating, ameliorating, and/or preventing cancer in a subject in need thereof, the method comprising administering to the subject an effective amount of a modified immune cell or precursor thereof comprising a chimeric antigen receptor (CAR) and an azido glycan and/or trans-cyclooctene group and/or tetrazine group, wherein the CAR is specific for a cancer-related antigen.

Embodiment 56 provides the method of embodiment 55, wherein further comprising administering to the subject a composition comprising an effective amount of a compound of formula (III) when the patient experiences a toxicity related to the administration of the modified immune cell.

Embodiment 57 provides the method of embodiment 56, wherein the compound of formula (III) is conjugated to the azido glycan and/or trans-cyclooctene group and/or tetrazine group on the surface of the cell in situ.

Embodiment 58 provides the method of embodiment 56 or 57, wherein administration of the compound of formula (III) reduces the interaction of the modified immune cells with endogenous immune cells such that the toxicity is resolved or reduced in severity

Embodiment 59 provides the method of any one of embodiments 55-58, wherein the toxicity is selected from the group consisting of cytokine release syndrome (CRS), CAR-T cell-related encephalopathy syndrome (CRES), cytokine release encephalopathy syndrome, immune effector cell associated neurotoxicity syndrome (ICANS), hemophagocytic lymphohistiocytosis/macrophage activation syndrome (HLH/MAS), and any combination thereof.

### OTHER EMBODIMENTS

The recitation of a listing of elements in any definition of a variable herein includes definitions of that variable as any single element or combination (or sub combination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A modified immune cell or precursor cell thereof comprising a chimeric antigen receptor (CAR) and one or more surface glycans,
wherein at least one of the surface glycans comprises a polysaccharide of formula (I): 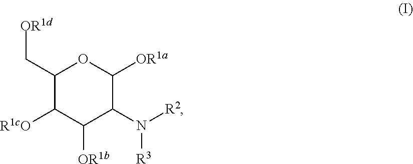
wherein:
R^{1a}, R^{1b}, R^{1c}, and R^{1d} are each independently selected from the group consisting of H, optionally substituted C₁-C₆ alkyl, optionally substituted C₂-C₆ heteroalkyl, optionally substituted C₃-C₈ cycloalkyl, optionally substituted phenyl, optionally substituted benzyl, optionally substituted C₂-C₉ heterocyclyl, C(═O)OR^{a}, C(═O)R^{a}, C(═O)N(R^{a})(R^{b}), S(═O)₂R^{a}, S(═O)₂N(R^{a})(R^{b}), a monosaccharide, a terminal polysaccharide, and a polysaccharide covalently conjugated to a CAR-T cell surface protein,
wherein one selected from R^{1a}, R^{1b}, R^{1c}, and R^{1d} is the polysaccharide covalently conjugated to the surface protein;
R² is -L¹—Y—L—²—Z;
R³ is selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
R⁴ is selected from the group consisting of optionally substituted C₁-C₆ alkyl, C₂-C₆ heteroalkyl, optionally substituted C₃-C₈ cycloalkyl, optionally substituted phenyl, optionally substituted benzyl, optionally substituted C₂-C₉ heterocyclyl, halogen, OR^{a}, N(R^{a})(R^{b}), SR^{a}, CN, and NO₂,
wherein two adjacent R⁴ substituents may combine with the atoms to which they are bound to form an optionally substituted phenyl, optionally substituted C₃-C₈ cycloalkyl, or optionally substituted C₂-C₉ heterocyclyl;
R⁵ is selected from the group consisting of H and optionally substituted C₁-C₆ alkyl;
R⁶ is selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
L¹ is selected from the group consisting of —C(═O)(optionally substituted C₁-C₆ alkylene)-*, —C(═O)(optionally substituted C₁-C₆ heteroalkylene)-*, -(optionally substituted C₁-C₆ alkylene)-*, and -(optionally substituted C₁-C₆ heteroalkylene)-*;
L² is selected from the group consisting of **—C(═O)(optionally substituted C₁-C₁₂ alkylene)C(═O)O—, **—C(═O)(optionally substituted C₂-C₁₂ heteroalkylene)C(═O)O—, **—C(═O)(optionally substituted C₁-C₁₂ alkylene)O—, **—C(═O)(optionally substituted C₂-C₁₂ heteroalkylene)O—, **-(optionally substituted C₁-C₁₂ alkylene)C(═O)O—, **-(optionally substituted C₂-C₁₂ heteroalkylene)C(═O)O—, **-(optionally substituted C₁-C₁₂ alkylene)O—, and **-(optionally substituted C₂-C₁₂ heteroalkylene)C(═O)O—;
Y is selected from the group consisting of 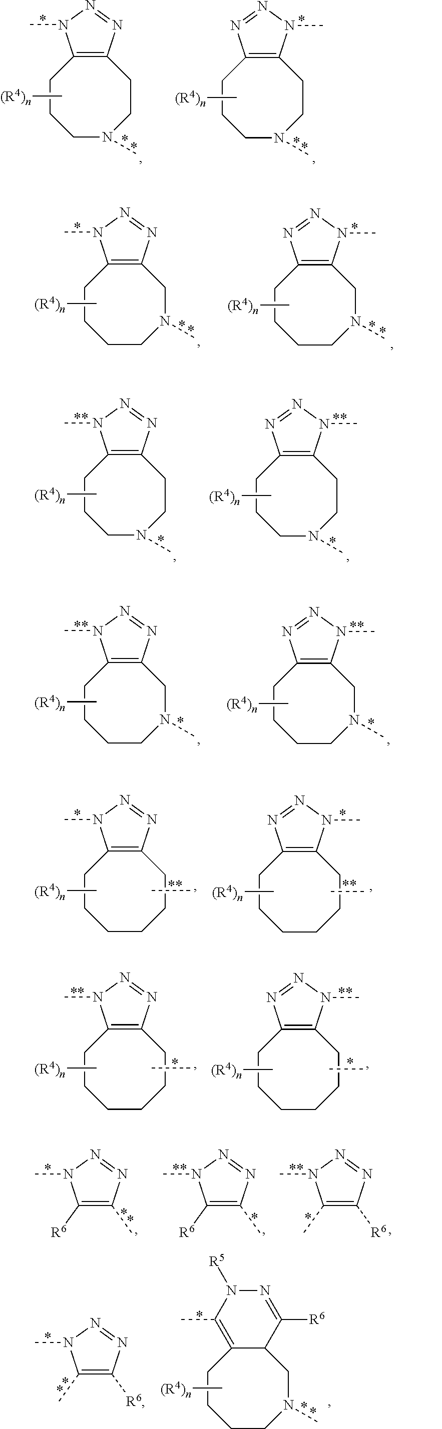 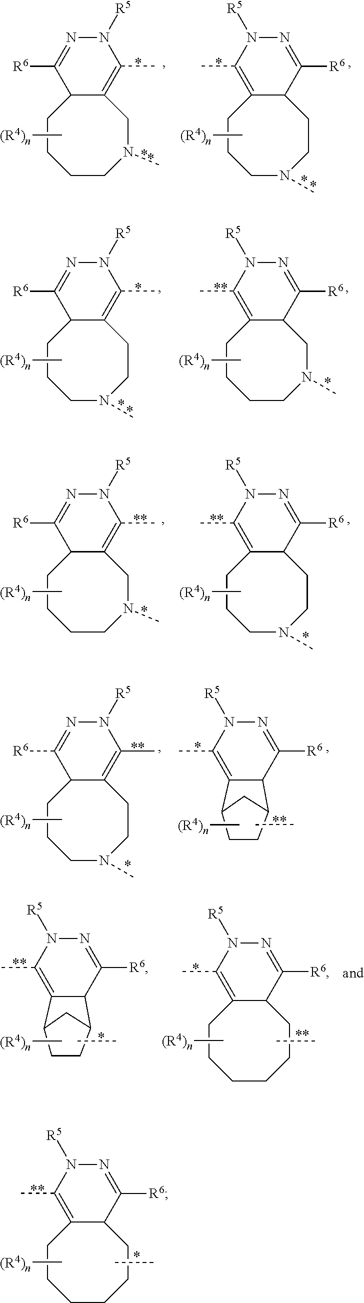
Z is selected from the group consisting of an optionally substituted heteroalkyl group, a dendrimer, a microparticle, a nanoparticle, an alignate, a biomolecule, and a modified red blood cell,
wherein the dendrimer, microparticle, nanoparticle, alignate, biomolecule, or modified red blood cell can be covalently linked to L² by an optionally substituted heteroalkylene, and
wherein each heteroalkyl or heteroalkylene has a molecular weight ranging from about 1 kDa to about 600 kDa;
each occurrence of R^{a} and R^{b} is independently selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
n is an integer ranging from 0 to 11;
* indicates a bond between L¹ and Y; and
** indicates a bond between Y and L².

2. The modified immune cell or precursor cell thereof of claim 1, wherein each occurrence of optionally substituted alkyl, optionally substituted heteroalkyl, optionally substituted cycloalkyl, optionally substituted alkoxy, optionally substituted phenyl, optionally substituted benzyl, optionally substituted heterocyclyl, optionally substituted alkylene, and optionally substituted heteroalkylene, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₉ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

3. The modified immune cell or precursor cell thereof of claim 1, wherein the compound of formula (I) is a compound of formula (Ia):

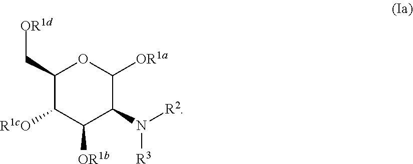

4. The modified immune cell or precursor cell thereof of claim 1, wherein one of the following applies:
(a) R^{1a} is the polysaccharide covalently conjugated to the surface protein, and each of R^{1b}, R^{1c}, and R^{1d} are C(═O)CH₃ or H;
(b) R^{1b} is the polysaccharide covalently conjugated to the surface protein, and each of R^{1a}, R^{1c}, and R^{1d} are C(═O)CH₃ or H;
(c) R^{1c} is the polysaccharide covalently conjugated to the surface protein, and each of R^{1a}, R^{1b}, and R^{1d} are C(═O)CH₃ or H;
(d) R^{1d} is the polysaccharide covalently conjugated to the surface protein, and each of R^{1a}, R^{1b}, and R^{1c} are C(═O)CH₃ or H;
(e) R^{1a} is the polysaccharide covalently conjugated to the surface protein, one of R^{1b}, R^{1c}, and R^{1d} is a monosaccharide or a terminal polysaccharide, and two of R^{1b}, R^{1c}, and R^{1d} are C(═O)CH₃ or H;
(f) R^{1b} is the polysaccharide covalently conjugated to the surface protein, one of R^{1a}R^{1c}, and R^{1d} is a monosaccharide or a terminal polysaccharide, and two of R^{1a}, R^{1c}, and R^{1d} are C(═O)CH₃ or H;
(g) R^{1c} is the polysaccharide covalently conjugated to the surface protein, one of R^{1a}, R^{1b}, and R^{1d} is a monosaccharide or a terminal polysaccharide, and two of R^{1a}, R^{1b}, and R^{1d} are C(═O)CH₃ or H; and
(h) R^{1d} is the polysaccharide covalently conjugated to the surface protein, one of R^{1a}R^{1b}, and R^{1c} is a monosaccharide or a terminal polysaccharide, and two of R^{1a}R^{1b}, and R^{1c} are C(═O)CH₃ or H.

5. The modified immune cell or precursor cell thereof of claim 1, wherein at least one of the following applies:
(a) R³ is H;
(b) each occurrence of R⁴ is H;
(c) R⁵ is H;
(d) R⁶ is H;
(e) L¹ is —C(═O)CH₂—*;
(f) L² is **—C(═O)(1,5-pentylene)C(═O)—.

6-10. (canceled)

11. The modified immune cell or precursor cell thereof of claim 1,
wherein Y is

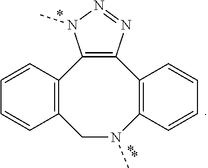

12. The modified immune cell or precursor cell thereof of claim 1, wherein at least one of the following applies:
(a) the optionally substituted heteroalkyl in Z is selected from the group consisting of a polyethylene glycol (PEG) polymer, a polyamidoamine (PAMAM) polymer, a polyethyleneimine (PEI) polymer, a polymethyl methacrylate (PMMA) polymer, a poly(N-isopropyleneimine) (PPI) polymer, and a polyvinyl alcohol (PVA) polymer,
optionally wherein the PEG polymer has the following formula: —(CH₂CH₂O)ₒ—H, wherein o is an integer ranging from 1 to 14,000:
(b) the heteroalkyl or heteroalkylene in Z has a molecular weight selected from the group consisting of about 1 kDa, about 5 kDa, about 10 kDa, about 100 kDa, and about 600 kDa.

13-14. (canceled)

15. The modified immune cell or precursor cell thereof of claim 1, wherein R² is selected from the group consisting of:

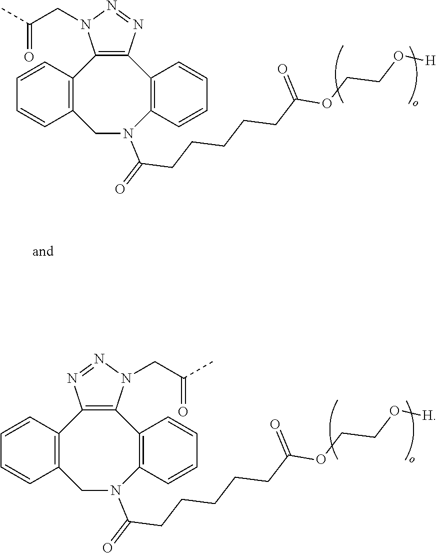

16. The modified immune cell or precursor thereof of claim 1, wherein at least one of the following applies:
(a) the surface glycan blocks cell-cell interactions by stearic hindrance,
(b) the chimeric antigen receptor comprises an antigen-binding domain capable of binding a tumor-associated antigen;
(c) the modified immune cell or precursor cell thereof is selected from the group consisting of an αβ T cell, a γδ T cell, a CD8 T cell, a CD4 helper T cell, a CD4 regulatory T cell, an NK T cell, an NK cell, and any combination thereof;
(d) the modified immune cell or precursor thereof is a T cell.

17-19. (canceled)

20. A method of preparing a modified immune cell or precursor cell thereof, the method comprising:
(a) contacting an immune cell or precursor cell thereof comprising a chimeric antigen receptor and further comprising one or more surface glycans, wherein at least one of the surface glycans comprises a polysaccharide of formula (II): 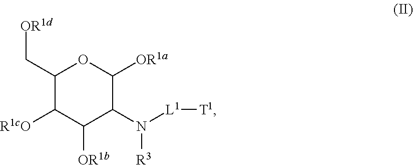
wherein:
R^{1a}, R^{1b}, R^{1c}, and R^{1d} are each independently selected from the group consisting of H, optionally substituted C₁-C₆ alkyl, optionally substituted C₂-C₆ heteroalkyl, optionally substituted C₃-C₈ cycloalkyl, optionally substituted phenyl, optionally substituted benzyl, optionally substituted C₂-C₉ heterocyclyl, C(═O)OR^{a}, C(═O)R^{a}, C(═O)N(R^{a})(R^{b}), S(═O)₂R^{a}, S(═O)₂N(R^{a})(R^{b}), a monosaccharide, a terminal polysaccharide, and a polysaccharide covalently conjugated to a CAR-T cell surface protein,
wherein one selected from R^{1a}, R^{1b}, R^{1c}, and R^{1d} is the polysaccharide covalently conjugated to the surface protein;
R³ is selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
R⁴ is selected from the group consisting of optionally substituted C₁-C₆ alkyl, C₂-C₆ heteroalkyl, optionally substituted C₃-C₈ cycloalkyl, optionally substituted phenyl, optionally substituted benzyl, optionally substituted C₂-C₉ heterocyclyl, halogen, OR^{a}, N(R^{a})(R^{b}), SR^{a}, CN, and NO₂,
wherein two adjacent R⁴ substituents may combine with the atoms to which they are bound to form an optionally substituted phenyl, optionally substituted C₃-C₈ cycloalkyl, or optionally substituted C₂-C₉ heterocyclyl;
R⁵ is selected from the group consisting of H and optionally substituted C₁-C₆ alkyl;
R⁶ is selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
L¹ is selected from the group consisting of —C(═O)(optionally substituted C₁-C₆ alkylene)-*, —C(═O)(optionally substituted C₁-C₆ heteroalkylene)-*, -(optionally substituted C₁-C₆ alkylene)-*, and -(optionally substituted C₁-C₆ heteroalkylene)-*;
T¹ is selected from the group consisting of N₃, 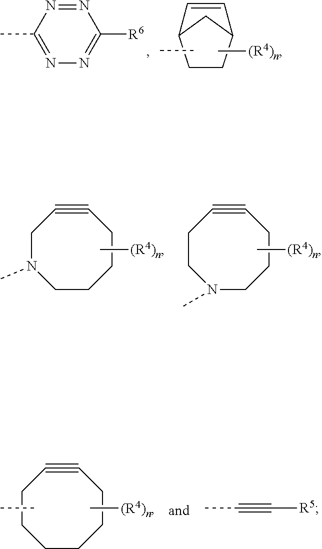
each occurrence of R^{a} and R^{b} is independently selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
n is an integer ranging from 0 to 11; and
(b) a compound of formula (III):
T²-L²-Z (III),
wherein:
T² is selected from the group consisting of —N₃, 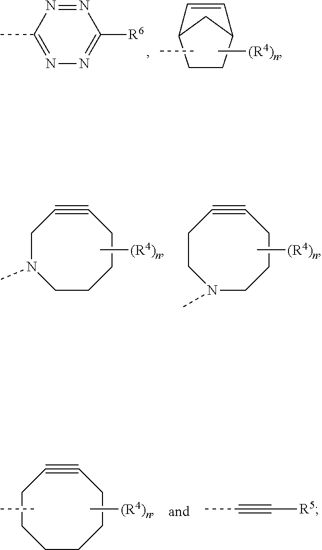
and
L² is selected from the group consisting of **—C(═O)(optionally substituted C₁-C₁₂ alkylene)C(═O)O—, **—C(═O)(optionally substituted C₂-C₁₂ heteroalkylene)C(═O)O—, **—C(═O)(optionally substituted C₁-C₁₂ alkylene)O—, **—C(═O)(optionally substituted C₂-C₁₂ heteroalkylene)O—, **-(optionally substituted C₁-C₁₂ alkylene)C(═O)O—, **-(optionally substituted C₂-C₁₂ heteroalkylene)C(═O)O—, **-(optionally substituted C₁-C₁₂ alkylene)O—, and **-(optionally substituted C₂-C₁₂ heteroalkylene)C(═O)O—;
Z is selected from the group consisting of an optionally substituted heteroalkyl group, a dendrimer, a microparticle, a nanoparticle, an alignate, a biomolecule, and a modified red blood cell,
wherein the dendrimer, microparticle, nanoparticle, alignate, biomolecule, or modified red blood cell can be covalently linked to L² by an optionally substituted heteroalkylene, and
wherein each heteroalkyl or heteroalkylene has a molecular weight ranging from about 1 kDa to about 600 kDa.

21. The method of claim 20, wherein one of the following applies:
(a) T¹ is selected from the group consisting of —N₃ and 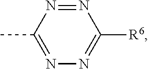
and T² is selected from the group consisting of 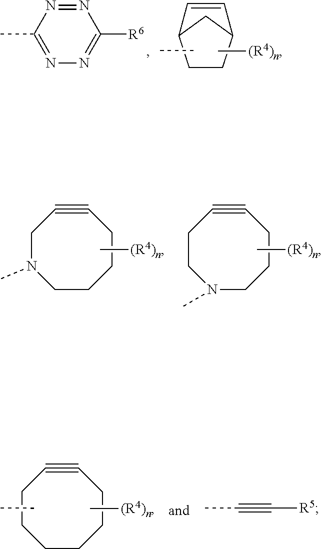
or
(b) T¹ is selected from the group consisting of 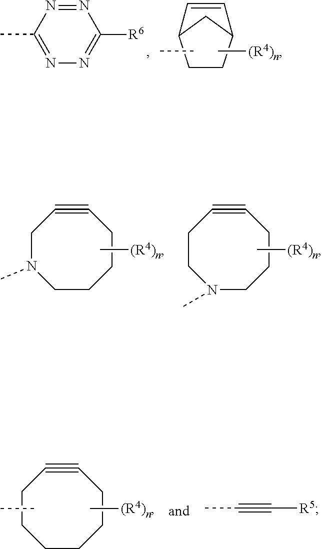
and T² is selected from the group consisting of —N₃ and

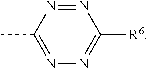

22. The method of claim 20, wherein the compound of formula (II) is a compound of formula (IIa):

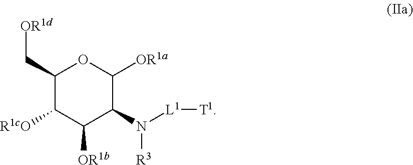

23. The method of claim 20, wherein one of the following applies:
(a) R^{1a} is the polysaccharide covalently conjugated to the surface protein, and each of R^{1b}, R^{1c}, and R^{1d} are C(═O)CH₃ or H;
(b) R^{1b} is the polysaccharide covalently conjugated to the surface protein, and each of R^{1a}, R^{1c}, and R^{1d} are C(═O)CH₃ or H;
(c) R^{1c} is the polysaccharide covalently conjugated to the surface protein, and each of R^{1a}, R^{1b}, and R^{1d} are C(═O)CH₃ or H;
(d) R^{1d} is the polysaccharide covalently conjugated to the surface protein, and each of R^{1a}, R^{1b}, and R^{1c} are C(═O)CH₃ or H;
(e) R^{1a} is the polysaccharide covalently conjugated to the surface protein, one of R^{1b}, R^{1c}, and R^{1d} is a monosaccharide or a terminal polysaccharide, and two of R^{1b}, R^{1c}, and R^{1d} are C(═O)CH₃ or H;
(f) R^{1b} is the polysaccharide covalently conjugated to the surface protein, one of R^{1a}R^{1c}, and R^{1d} is a monosaccharide or a terminal polysaccharide, and two of R^{1a}R^{1c}, and R^{1d} are C(═O)CH₃ or H;
(g) R^{1c} is the polysaccharide covalently conjugated to the surface protein, one of R^{1a}R^{1b}, and R^{1d} is a monosaccharide or a terminal polysaccharide, and two of R^{1a}R^{1b}, and R^{1d} are C(═O)CH₃ or H; and
(h) R^{1d} is the polysaccharide covalently conjugated to the surface protein, one of R^{1a}R^{1b}, and R^{1c} is a monosaccharide or a terminal polysaccharide, and two of R^{1a}R^{1b}, and R^{1c} are C(═O)CH₃ or H.

24. The method of claim 20, wherein at least one of the following applies:
(a) R³ is H;
(b) each occurrence of R⁴ is H;
(c) R⁵ is H;
(d) R⁶ is H;
(e) L¹ is —C(═O)CH₂—*;
(f) L² is **—C(═O)(1,5-pentvlene)C(═O)—.

25-29. (canceled)

30. The method of claim 20, wherein at least one of the following applies:
(a) the optionally substituted heteroalkyl in Z is selected from the group consisting of a polyethylene glycol (PEG) polymer, a polyamidoamine (PAMAM) polymer, a polyethyleneimine (PEI) polymer, a polymethyl methacrylate (PMMA) polymer, a poly(N-isopropyleneimine) (PPI) polymer, and a polyvinyl alcohol (PVA) polymer,
optionally wherein the PEG polymer has the following formula: —(CH₂CH₂O)ₒ—H, wherein o is an integer ranging from 1 to 14,000;
(b) the heteroalkyl or heteroalkylene in Z has a molecular weight selected from the group consisting of about 1 kDa, about 5 kDa, about 10 kDa, about 100 kDa, and about 600 kDa.

31-32. (canceled)

33. The method of claim 20, wherein T¹ is N₃.

34. The method of claim 20, wherein T² is

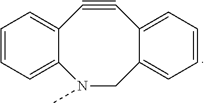

35. The method of claim 20, wherein the compound of formula (III) is:

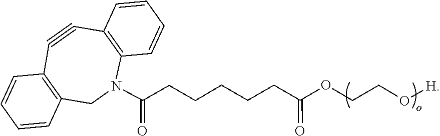

36. The method of claim 20, wherein at least one of the following applies:
(a) the chimeric antigen receptor comprises antigen binding domain capable of binding a tumor-associated antigen,
(b) the modified immune cell or precursor cell thereof is selected from the group consisting of an αβ T cell, a γδ T cell, a CD8 T cell, a CD4 helper T cell, a CD4 regulatory T cell, an NK T cell, an NK cell, and any combination thereof;
(c) the modified immune cell or precursor thereof is a T cell.

37-38. (canceled)

39. A method for reducing the severity of a toxicity in a subject caused by administration of a chimeric antigen receptor (CAR) expressing T cell to the subject, said method comprising:
a. labeling the CAR expressing T cell with an azido glycan or trans-cyclooctene group and/or tetrazine group, and
b. conjugating a spacer molecule to the azido glycan or trans-cyclooctene group and/or tetrazine group, thereby producing a labeled CAR T cell;
wherein the conjugated spacer molecule reduces the interaction of the CAR T cell with endogenous immune cells, thereby reducing the severity of the toxicity.

40. The method of claim 39, wherein the spacer molecule is selected from the group consisting of an optionally substituted heteroalkyl group, a dendrimer, a microparticle, a nanoparticle, an alginate, a biomolecule, and a modified red blood cell,
wherein the dendrimer, microparticle, nanoparticle, alginate, biomolecule, or modified red blood cell can be covalently linked to the T cell by an optionally substituted heteroalkylene, and
wherein each heteroalkyl or heteroalkylene has a molecular weight ranging from about 1 kDa to about 600 kDa.

41. The method of claim 40, wherein at least one of the following applies:
(a) the optionally substituted heteroalkyl is selected from the group consisting of a polyethylene glycol (PEG) polymer, a polyamidoamine (PAMAM) polymer, a polyethyleneimine (PEI) polymer, a polymethyl methacrylate (PMMA) polymer, a poly(N-isopropyleneimine) (PPI) polymer, and a polyvinyl alcohol (PVA) polymer,
optionally wherein the PEG polymer has the following formula: —(CH₂CH₂O)ₒ—H, wherein o is an integer ranging from 1 to 14,000;
(b) the heteroalkyl or heteroalkylene has a molecular weight selected from the group consisting of about 1 kDa, about 5 kDa, about 10 kDa, about 100 kDa, and about 600 kDa.

42-43. (canceled)

44. The method of claim 39, wherein at least one of the following applies:
(a) the labeling of the CAR T cell with an azido glycan or trans-cyclooctene group or tetrazine group occurs ex vivo from the subject,
(b) the spacer molecule is a compound of formula (III):
T²-L²-Z (III),
wherein:
T² is selected from the group consisting of —N₃, 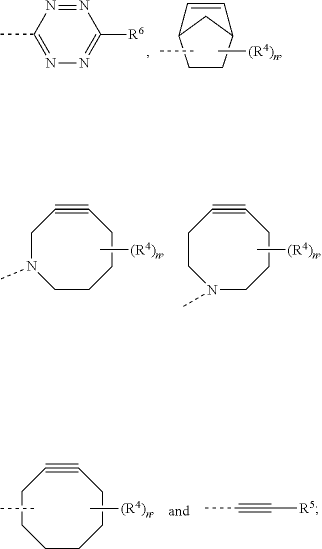
and
L² is selected from the group consisting of **—C(═O)(optionally substituted C₁-C₁₂ alkylene)C(═O)O—, **—C(═O)(optionally substituted C₂-C₁₂ heteroalkylene)C(═O)O—, **—C(═O)(optionally substituted C₁-C₁₂ alkylene)O—, **—C(═O)(optionally substituted C₂-C₁₂ heteroalkylene)O—, **-(optionally substituted C₁-C₁₂ alkylene)C(═O)O—, **-(optionally substituted C₂-C₁₂ heteroalkylene)C(═O)O—, **-(optionally substituted C₁-C₁₂ alkylene)O—, and **-(optionally substituted C₂-C₁₂ heteroalkylene)C(═O)O—;
Z is selected from the group consisting of an optionally substituted heteroalkyl group, a dendrimer, a microparticle, a nanoparticle, an alignate, a biomolecule, and a modified red blood cell,
wherein the dendrimer, microparticle, nanoparticle, alignate, biomolecule, or modified red blood cell can be covalently linked to L² by an optionally substituted heteroalkylene, and
wherein each heteroalkyl or heteroalkylene has a molecular weight ranging from about 1 kDa to about 600 kDa:
(c) an effective amount of the spacer molecule is administered to the subject such that the conjugation of the space molecule occurs in vivo in the subject;
(d) the conjugation of the spacer molecule occurs after administering the CAR T cell to the subject;
(e) the spacer molecule administration occurs after the administration of the CAR T cell to the subject;
(f) the azido glycan is N-azidoacetylmannosamine-tetraacylated (Ac4ManNAz);
(g) the PEG is PEG 600 k;
(h) the reduction of the interaction of the CAR T cell with endogenous immune cells occurs by steric hindrance;
(i) the toxicity is selected from the group consisting of cytokine release syndrome (CRS), CAR-T cell-related encephalopathy syndrome (CRES), cytokine release encephalopathy syndrome, immune effector cell associated neurotoxicity syndrome (ICANS), hemophagocytic lymphohistiocytosis/macrophage activation syndrome (HLH/MAS), and any combination thereof;
(j) the effective amount of the spacer molecule is administered to the subject when the subject presents at least one symptom associated with the toxicity,
optionally wherein the symptom associated with the toxicity is selected from the group consisting of high fever, rigors, malaise, fatigue, myalgia, nausea, anorexia, tachycardia/hypotension, hypotension, headache, aphasia, disorientation, lethargy, capillary leak, cardiac dysfunction, renal impairment, hepatic failure, and disseminated intravascular coagulation, elevated IL-6 levels, elevated IL-5 levels, elevated IL-13 levels, elevated IL-10 levels, elevated interferon-gamma (IFNγ) levels dermatitis, tachycardia, hypotension, headache, nausea, aphasia, disorientation, lethargy, and any combination thereof.

45-54. (canceled)

55. A method of treating, ameliorating, or preventing cancer in a subject in need thereof, the method comprising administering to the subject an effective amount of a modified immune cell or precursor thereof comprising a chimeric antigen receptor (CAR) and an azido glycan or trans-cyclooctene group or tetrazine group, wherein the CAR is specific for a cancer-related antigen.

56. The method of claim 55, further comprising administering to the subject a composition comprising an effective amount of a compound of formula (III) when the patient experiences a toxicity related to the administration of the modified immune cell:
T²-L²-Z (III),
wherein:
T² is selected from the group consisting of —N₃, 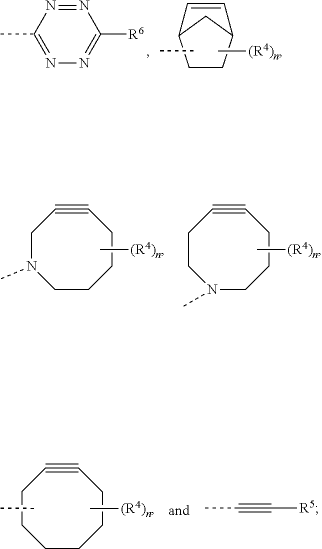
and
L² is selected from the group consisting of **—C(═O)(optionally substituted C₁-C₁₂ alkylene)C(═O)O—, **—C(═O)(optionally substituted C₂-C₁₂ heteroalkylene)C(═O)O—, **—C(═O)(optionally substituted C₁-C₁₂ alkylene)O—, **—C(═O)(optionally substituted C₂-C₁₂ heteroalkylene)O—, **-(optionally substituted C₁-C₁₂ alkylene)C(═O)O—, **-(optionally substituted C₂-C₁₂ heteroalkylene)C(═O)O—, **-(optionally substituted C₁-C₁₂ alkylene)O—, and **-(optionally substituted C₂-C₁₂ heteroalkylene)C(═O)O—;
Z is selected from the group consisting of an optionally substituted heteroalkyl group, a dendrimer, a microparticle, a nanoparticle, an alignate, a biomolecule, and a modified red blood cell,
wherein the dendrimer, microparticle, nanoparticle, alignate, biomolecule, or modified red blood cell can be covalently linked to L² by an optionally substituted heteroalkylene, and
wherein each heteroalkyl or heteroalkylene has a molecular weight ranging from about 1 kDa to about 600 kDa.

57. The method of claim 56, wherein at least one of the following applies:
(a) the compound of formula (III) is conjugated to the azido glycan and/or trans-cyclooctene group and/or tetrazine group on the surface of the cell in situ,
(b) administration of the compound of formula (III) reduces the interaction of the modified immune cells with endogenous immune cells such that the toxicity is resolved or reduced in severity;
(c) the toxicity is selected from the group consisting of cytokine release syndrome (CRS), CAR-T cell-related encephalopathy syndrome (CRES), cytokine release encephalopathy syndrome, immune effector cell associated neurotoxicity syndrome (ICANS), hemophagocytic lymphohistiocytosis/macrophage activation syndrome (HLH/MAS), and any combination thereof.

58-59. (canceled)
